(19) United States
(12) Patent Application Publication
(10) Pub. No.: US 2024/0200066 A1
(43) Pub. Date: Jun. 20, 2024

(54) PRODUCTS AND METHODS FOR TREATING MUSCULAR DYSTROPHY

(71) Applicant: RESEARCH INSTITUTE AT NATIONWIDE CHILDREN'S HOSPITAL, Columbus, OH (US)

(72) Inventors: Nicolas Sebastien Wein, Columbus, OH (US); Kevin Flanigan, Columbus, OH (US)

(21) Appl. No.: 18/287,677

(22) PCT Filed: Apr. 22, 2022
(86) PCT No.: PCT/US22/25986

§ 371 Date: Oct. 20, 2023

Related U.S. Application Data
(60) Provisional application No. 63/178,648, filed on Apr. 23, 2021.

(51) Int. Cl. C12N 15/113; A61K 9/00; A61K 31/573; A61K 38/17; A61K 45/06; A61P 21/00; C12N 15/86; G01N 33/68

(52) U.S. Cl. CPC C12N 15/113; A61K 9/0019; A61K 31/573; A61K 38/1719; A61K 45/06; A61P 21/00; C12N 15/86; G01N 33/6854; G01N 33/6893; C12N 2310/11; C12N 2750/14143; G01N 2333/4712

(57) ABSTRACT

Products and methods for treating or preventing muscular dystrophies in patients with mutations in any of exons 6, 7, 8, or 9 in their DMD gene are provided. Gene therapy vectors, such as adeno-associated virus (AAV) vectors, and methods of using these vectors to deliver nucleic acids comprising DMD antisense sequences in regulating or restoring expression of transcripts of the DMD gene and a functional form of the dystrophin protein are provided. The products and methods are used for treating, ameliorating and/or preventing muscular dystrophies, such as Duchenne Muscular Dystrophy or Becker Muscular Dystrophy.

### CROSS-REFERENCE TO RELATED APPLICATIONS

The present application claims the priority benefit of U.S. Provisional Patent Application No. 63/178,648, filed Apr. 23, 2021, hereby incorporated by reference in its entirety.

### FIELD

The disclosure relates to the field of gene therapy for the treatment and/or prevention of muscular dystrophy. More particularly, the disclosure provides products and methods for treating or preventing muscular dystrophies in patients with resulting from mutations in DMD exons 6, 7, 8, and/or 9. The disclosure provides nucleic acids comprising nucleotide sequences for antisense-mediated exon-skipping to skip frame-disrupting exon(s) and allow functional dystrophin protein expression by restoring the reading frame. Gene therapy vectors, such as adeno-associated virus (AAV) vectors, comprising the nucleic acids and methods of using these vectors to express the dystrophin gene and protein are provided. The products and methods are used for treating and/or preventing muscular dystrophies, such as Duchenne Muscular Dystrophy or Becker Muscular Dystrophy.

### INCORPORATION BY REFERENCE OF THE SEQUENCE LISTING

This application contains, as a separate part of disclosure, a Sequence Listing in computer-readable form (Filename: 56493_SeqListing.txt; 20,439 bytes—ASCII text file created Apr. 22, 2022) which is incorporated by reference herein in its entirety.

### BACKGROUND

Muscular dystrophies (MDs) are a group of genetic degenerative diseases primarily affecting voluntary muscles. The group is characterized by progressive weakness and degeneration of the skeletal muscles that control movement. Some forms of MD develop in infancy or childhood, while others may not appear until middle age or later. The disorders differ in terms of the distribution and extent of muscle weakness (some forms of MD also affect cardiac muscle), the age of onset, the rate of progression, and the pattern of inheritance.

The MDs are a group of diseases without identifiable treatment that gravely impact individuals, families, and communities. The costs are incalculable. Individuals suffer emotional strain and reduced quality of life associated with loss of self-esteem. Extreme physical challenges resulting from loss of limb function creates hardships in activities of daily living. Family dynamics suffer through financial loss and challenges to interpersonal relationships. Siblings of the affected feel estranged, and strife between spouses often leads to divorce, especially if responsibility for the muscular dystrophy can be laid at the feet of one of the parental partners. The burden of quest to find a cure often becomes a life-long, highly focused effort that detracts and challenges every aspect of life. Beyond the family, the community bears a financial burden through the need for added facilities to accommodate the handicaps of the muscular dystrophy population in special education, special transportation, and costs for recurrent hospitalizations to treat recurrent respiratory tract infections and cardiac complications. Financial responsibilities are shared by state and federal governmental agencies extending the responsibilities to the taxpaying community.

One form of MD is Duchenne Muscular Dystrophy (DMD). DMD, an X-linked degenerative muscle disorder, is the most common severe childhood form of muscular dystrophy affecting around 1:5200 male births (Mendell et al., Ann Neurol 71, 304-313 (2012)). Symptoms of generalized muscle weakness first appear at ages 3-5 and progress into a loss of ambulation by age 13, with death typically occurring in the third decade of life due to cardiomyopathy or respiratory insufficiency (Passamano et al., Acta Myol 31, 121-125 (2012); Duchenne, The Pathology of Paralysis with Muscular Degeneration (Paralysie Myosclerotique), or Paralysis with Apparent Hypertrophy. Br Med J 2, 541-542 (1867)). DMD is caused by mutations that disrupt the open reading frame in the DMD gene, which encodes dystrophin (Juan-Mateu et al., PLOS One 10, e0135189 (2015)), a large (427 kDa) multifunctional protein that is localized at the subsarcolemmal region of myofibers, where it plays an important role in protecting the sarcolemma from mechanical damage caused by muscle contraction (Petrof et al., Proc Natl Acad Sci USA 90, 3710-3714 (1993)).

Another form of MD is Becker Muscular Dystrophy (BMD). BMD is a milder allelic disorder which results from the presence of a partially functional dystrophin protein occurring from mutations that maintain an open reading frame (ORF) (Wein et al., Nature Medicine 20, 992-1000 (2014); Monaco, Trends Biochem Sci 14, 412-415 (1989)). BMD, like DMD, is a genetic disorder that gradually makes the body's muscles weaker and smaller. BMD affects the muscles of the hips, pelvis, thighs, and shoulders, as well as the heart, but is known to cause less severe problems than DMD. Because of the variety of in-frame mutations resulting in a variety of partially functional proteins, BMD has a broad phenotypic spectrum with, for example, loss of ambulation ranging from the late teenage years to late adulthood.

Promising therapeutic approaches to DMD are based on the replacement of a functional version of DMD, or its repair at the DNA or pre-mRNA level. Both approaches aim at restoration of an open reading frame, leading to expression of a partially function, BMD-like dystrophin. Gene replacement trials using modified adeno-associated viruses (AAVs) have been reported (Muzyczka, Curr Top Microbiol Immunol 158, 97-129 (1992); Carter, Mol Ther 10, 981-989 (2004); Samulski et al., Annu Rev Virol 1, 427-451 (2014)), but transgene packaging capacity of AAV is limited to ˜5 kb. Because the DMD cDNA is 11.4 kb, current viral vectors make use of one of several internally-deleted but in-frame microdystrophin CDNAs (Duan, Mol Ther 26, 2337-2356 (2018)). An alternate approach is to restore the mRNA reading frame by delivering an antisense sequence that binds to key exon definition elements in the pre-mRNA, inhibiting the recognition of a specific exon by the spliceosome, leading to exclusion of the target exon from the mature RNA. Such antisense sequences can consist of antisense oligonucleotides (AONs), or phoshphorodiamidate morpholino oligomers (PMO), such as eteplirsen, the first such therapy approved by the FDA for treatment of DMD due to mutations amenable to skipping of exon 51 (Barthelemy et al., Neuromuscul Disord 28, 803-824 (2018); Wein et al., Pediatr Clin North Am 62, 723-742 (2015); Alfano et al., Medicine (Baltimore) 98, e15858 (2019)).

Despite many lines of research following the identification of the DMD gene, treatment options are limited. There thus remains a need in the art for treatments for MDs, including DMD and BMD, including treatments for one or more mutations of the DMD gene.

### SUMMARY

The disclosure provides products and methods for preventing disease, delaying the progression or severity of disease, and/or treating disease in patients with one or more mutations of exons 6, 7, 8, and/or 9 of the DMD gene. More particularly, the disclosure provides products and methods using a U7snRNA approach to induce skipping of exons 6, 7, 8, and/or 9 of the DMD gene.

More particularly, the disclosure provides products and methods for treating or preventing muscular dystrophies in patients with resulting from mutations in DMD exons 6, 7, 8, and/or 9. The disclosure provides nucleic acids comprising nucleotide sequences for antisense-mediated exon-skipping to skip frame-disrupting exon(s) and allow functional dystrophin protein expression by restoring the reading frame. Gene therapy vectors, such as adeno-associated virus (AAV) vectors, comprising the nucleic acids and methods of using these vectors to express the dystrophin gene and protein are provided. The products and methods are used for treating and/or preventing muscular dystrophies, such as Duchenne Muscular Dystrophy or Becker Muscular Dystrophy.

The disclosure provides a nucleic acid comprising a nucleotide sequence or a combination of nucleotide sequences designed for targeting one or more of exons 6, 7, and 8 of the human DMD gene in order to effect antisense-mediated exon skipping and treat, ameliorate, or prevent muscular dystrophies in patients resulting from mutations in DMD exons 6, 7, 8, and/or 9.

The disclosure provides a nucleic acid comprising a nucleotide sequence selected from the group consisting of:
(a) a nucleotide sequence comprising at least 80% identity to the sequence set forth in any one of SEQ ID NOs: 1-20;
(b) a nucleotide sequence complementary to the nucleotide sequence comprising at least 80% identity to the sequence set forth in any one of SEQ ID NOs: 1-20;
(c) a nucleotide sequence comprising the sequence set forth in any one of SEQ ID NOs: 1-20;
(d) a nucleotide sequence complementary to the nucleotide sequence comprising the sequence set forth in any one of SEQ ID NOs: 1-20; and
(e) a nucleotide sequence which binds to the sequence set forth in any one of SEQ ID NOs: 21-25.

In some aspects, the nucleic acid comprises a combination of at least two nucleotide sequences, wherein the combination comprises
(i) a nucleotide sequence that targets the human DMD gene at exon 6 and a nucleotide sequence that targets the human DMD gene at exon 7,
(ii) a nucleotide sequence that targets the human DMD gene at exon 6 and a nucleotide sequence that targets the human DMD gene at exon 8, and
(iii) a nucleotide sequence that targets the human DMD gene at exon 7 and a nucleotide sequence that targets the human DMD gene at exon 8.

In some aspects, the nucleic acid comprises a combination of at least three nucleotide sequences, wherein the combination comprises a nucleotide sequence that targets the human DMD gene at exon 6, a nucleotide sequence that targets the human DMD gene at exon 7, and a nucleotide sequence that targets the human DMD gene at exon 8.

In some aspect, the nucleic acid comprises a nucleotide sequence selected from the group consisting of:
(a) a nucleotide sequence comprising at least 70% identity to the sequence set forth in any one of SEQ ID NOs: 26-29;
(b) a nucleotide sequence complementary to the nucleotide sequence comprising at least 70% identity to the sequence set forth in any one of SEQ ID NOs: 26-29;
(c) a nucleotide sequence comprising the sequence set forth in any one of SEQ ID NOs: 26-29; and
(d) a nucleotide sequence complementary to the nucleotide sequence comprising the sequence set forth in any one of SEQ ID NOs: 26-29.

The disclosure provides a recombinant adeno-virus associated (rAAV) comprising any of the nucleic acids described herein. In some aspects, the rAAV is rAAV1, rAAV2, rAAV3, rAAV4, rAAV5, rAAV6, rAAV7, rAAV8, rAAV9, rAAV10, rAAV11, rAAV12, rAAV13, rAAV-anc80, rAAV rh.74, rAAV rh.8, rAAVrh.10, or rAAV-B1. In some aspects, the rAAV is rAAV9. In some aspects, the rAAV is self-complementary.

The disclosure provides a composition comprising any one or more of the nucleic acids described herein and a carrier, diluent, excipient, and/or adjuvant.

The disclosure provides a composition comprising any vector described herein and a carrier, diluent, excipient, and/or adjuvant. In some aspects, the vector is AAV or rAAV.

The disclosure provides a method of treating, preventing or ameliorating a muscular dystrophy in a subject in need thereof comprising the step of administering to the subject an effective amount of
(a) any one or more of the nucleic acids described herein;
(b) any vector described herein; or
(c) any composition comprising the nucleic acids or vectors described herein.

In some aspects, the vector is AAV or rAAV. In some aspects, the rAAV is rAAV1, rAAV2, rAAV3, rAAV4, rAAV5, rAAV6, rAAV7, rAAV8, rAAV9, rAAV10, rAAV11, rAAV12, rAAV13, rAAV-anc80, rAAV rh.74, rAAV rh.8, rAAVrh.10, or rAAV-B1. In some aspects, the rAAV is rAAV9. In some aspects, the rAAV is self-complementary. In some aspects, the administering is via a systemic route. In some aspects, the systemic route is by injection, infusion or implantation. In some aspects, the muscular dystrophy is Duchenne Muscular Dystrophy or Becker Muscular Dystrophy.

In some aspects of the method, the level of functional dystrophin gene expression or protein expression in a cell of the subject is increased after administering the nucleic acid, rAAV, or composition as compared to the level of functional dystrophin gene expression or protein expression before administering the nucleic acid, rAAV, or composition.

In some aspects of the method, the expression of functional dystrophin in the cell is detected by measuring the dystrophin protein level by Western blot, immunofluorescence, or immunohistochemistry in muscle biopsied before and after administering the nucleic acid, rAAV, or composition.

In some aspects of the method, the level of serum creatinine kinase is decreased after administering the nucleic acid, rAAV, or composition as compared to the level of serum creatinine kinase before administering the nucleic acid, rAAV, or composition.

In some aspects of the method, the treatment results in improved muscle strength, improved muscle function, improved mobility, improved stamina, or a combination of two or more thereof in the subject.

In some aspects of the method, the muscular dystrophy progression in the subject is delayed or wherein muscle function in the subject is improved after administering the nucleic acid, rAAV, or composition as measured by the six minute walk test, time to rise test, ascend 4 steps test, ascend and descend 4 steps test, North Star Ambulatory Assessment (NSAA), the forced vital capacity (FVC) test, 10 meter timed test, 100 meter timed test, hand held dynamometry (HHD) test, Timed Up and Go test, Gross Motor Subtest Scaled (Bayley-III) score, maximum isometric voluntary contraction test (MVICT), or a combination of two or more thereof.

In some aspects, a method of treatment of the disclosure further comprises administering a second or combination therapy. In some aspects, the method further comprises administering a glucocorticoid.

The disclosure also provides use of
(a) any one or more of the nucleic acids described herein;
(b) any vector described herein; or
(c) any composition comprising the nucleic acids or vectors described herein
   for the preparation of a medicament for the treatment of a muscular dystrophy, or for treating a muscular dystrophy in a human subject in need thereof.

In some aspects, the vector is AAV or rAAV. In some aspects, the rAAV is rAAV1, rAAV2, rAAV3, rAAV4, rAAV5, rAAV6, rAAV7, rAAV8, rAAV9, rAAV10, rAAV11, rAAV12, rAAV13, rAAV-anc80, rAAV rh.74, rAAV rh.8, rAAVrh.10, or rAAV-B1. In some aspects, the rAAV is rAAV9. In some aspects, the rAAV is self-complementary. In some aspects, the medicament is designed for use via a systemic route. In some aspects, the systemic route is by injection, infusion or implantation. In some aspects, the muscular dystrophy is Duchenne Muscular Dystrophy or Becker Muscular Dystrophy.

In some aspects of the use, the level of functional dystrophin gene expression or protein expression in a cell of the subject is increased after administering the nucleic acid, rAAV, or composition as compared to the level of functional dystrophin gene expression or protein expression before administering the nucleic acid, rAAV, or composition.

In some aspects of the use, the expression of functional dystrophin in the cell is detected by measuring the dystrophin protein level by Western blot, immunofluorescence, or immunohistochemistry in muscle biopsied before and after administering the nucleic acid, rAAV, or composition.

In some aspects of the use, the level of serum creatinine kinase is decreased after administering the nucleic acid, rAAV, or composition as compared to the level of serum creatinine kinase before administering the nucleic acid, rAAV, or composition.

In some aspects of the use, the treatment results in improved muscle strength, improved muscle function, improved mobility, improved stamina, or a combination of two or more thereof in the subject.

In some aspects of the use, the muscular dystrophy progression in the subject is delayed or wherein muscle function in the subject is improved after administering the nucleic acid, rAAV, or composition as measured by the six minute walk test, time to rise test, ascend 4 steps test, ascend and descend 4 steps test, North Star Ambulatory Assessment (NSAA), the forced vital capacity (FVC) test, 10 meter timed test, 100 meter timed test, hand held dynamometry (HHD) test, Timed Up and Go test, Gross Motor Subtest Scaled (Bayley-III) score, maximum isometric voluntary contraction test (MVICT), or a combination of two or more thereof.

In some aspects, a use of the disclosure further comprises a second or combination therapy. In some aspects, the use further comprises administering a glucocorticoid.

Other features and advantages of the disclosure will become apparent from the following description of the drawing and the detailed description. It should be understood, however, that the drawing, detailed description, and the examples, while indicating aspects of the disclosed subject matter, are given by way of illustration only, because various changes and modifications within the spirit and scope of the disclosure will become apparent from the drawing, detailed description, and the examples.

### BRIEF DESCRIPTION OF THE DRAWINGS

FIG. 1A-F shows the comparison of nucleotide sequences of DMD exons 6, 7, and 8 in human, mouse and dog. FIG. 1 also shows the localization of the antisense sequences targeting those exons and the targeted sequences. The targeted sequence is underlined. The highlighted and bolded bases show differences in the mouse and dog sequences compared to the human sequence (lower case=intron sequence; upper case=exon sequence). FIG. 1A shows human, mouse, and dog exon 6 sequences. FIG. 1B shows target and antisense sequences for exon 6. FIG. 1C shows human, mouse, and dog exon 7 sequences. FIG. 1D shows target and antisense sequences for exon 7. FIG. 1E shows human mouse and dog exon 8 sequences. FIG. 1F shows target and antisense sequences for exon 8.

FIG. 2A-C shows how a combination of antisense sequences targeting exons 6, 7, and/or 8 can mediate skipping of exons 6-8 in WT FibroMyoD. FIG. 2A shows RT-PCR results from transduction of WT FibroMyoD using a combination of antisense sequences (as set out in the panel to the right) in AAV1 (2.5E11vg/⁶ cm well). Primers amplifying WT and skipped transcripts were located in exon 5 and 10. All different constructs led to multi-exon skipping. Exon 9 is often naturally skipped as displayed in the untreated control cell line. Several bands were obtained after treatment including two bands for two transcripts: (1) a transcript comprising exons 5, 9, 10 (top box comprising two transcripts; outlined in red; from treatment with ESE8_2, ESE7 and published antisense against exon 6 (i.e., ESE6, as published by Vulin et al., Mol. Ther. 2012 November; 20(11):2120-33. doi: 10.1038/mt.2012.181. Epub 2012 Sep. 11. PMID: 22968479; Bish et al., Mol. Ther. 2012 March; 20(3):580-9. doi: 10.1038/mt.2011.264. Epub 2011 Dec. 6. PMID: 22146342); or treatment with ESE8_2, ESE7, and SD6); and (2) a transcript comprising exons 5 and 10 (bottom box comprising 8 transcripts, outlined in green; from treatment with various combinations of antisense as set out in the panel to the right). Both transcripts, comprising exons 5, 9, and 10 and exons 5 and 10 are therapeutic as they restore the reading frame of DMD. FIG. 2B shows Sanger sequencing of transcripts from the bottom box in green corresponding to the exons 5, 10 transcript. FIG. 2C shows Sanger sequencing of transcripts from the top box in red corresponding to the exon 5, 9, 10 transcript.

FIG. 3A-D illustrates the U7snRNA vector approach to exon skipping. U7snRNA is used as a carrier to target the pre-messenger RNA. It is composed of a loop used for the nucleocytoplasmic export, a recognition sequence to bind the Sm proteins used for an efficient assembly between the U7snRNA and the target pre-mRNA and an antisense sequence to target the pre-mRNA. It has its own promoter and 3′ downstream sequences. The U7 cassette is then cloned into an AAV plasmid to produce the vector. Two orientations are represented as example: a forward construct containing U7-antisense sequence targeting exons 6, 7 and 8 (FIG. 3A and 3C; U7 ESE8_2_ESE7_SD6_forward) and a reverse orientation of a similar construct (FIG. 3B and 3D; U7 ESE8_2_ESE7_SD6_reverse). Nucleic acids comprising SEQ ID NOs: 26 and 28 are represented in FIG. 3A-D. Lengths of exon and intron are presented in blue arrows and in addition number in red corresponds to amino acid. PCR amplicons from exons 5-10 and 5-9 differ by 129 bp.

FIG. 4A-D shows the generation and characterization of a new mouse model, the hDMDm7 model, carrying a nonsense mutation in exon 7 of the hDMD gene. FIG. 4A shows a schematic of human DMD exons 6, 7 and 8. The lightening symbol represents where the CRISPR/Cas9 complex cuts the human DMD exon 7. FIG. 4B shows Sanger sequence confirmation of an introduced nonsense mutation following genome editing. FIG. 4C shows RT-PCR results from the hDMDm7 mouse model, confirming the presence of hDMD transcript despite the nonsense mutation at both 4 weeks and three months. As expected, a transcript without exon 9 was also detected (bottom band). FIG. 4D shows Western blot results from control (BI6 mice) and hDMDm7 mice. Dystrophin is present in the WT mouse but is absent in the hDMDm7 mouse (C-terminal antibody: PA1-21011, Thermo Fisher). Full-length Dystrophin (top arrow); loading control (bottom arrow).

FIG. 5A-C shows muscle histopathology and force generation of the tibialis anterior (TA) muscle of the transgenic mouse model of DMD (hDMDm7 or delCH2) compared to the control mouse (hDMD). FIG. 5A shows H&E staining performed on tibialis anterior (TA), gastrocnemius (Gas), triceps (Tri), and diaphragm (Dia) muscles from WT hDMD and hDMDm7 (delCH2) mice at 3 months and 6 months of age (n=5). In both 3 and 6-month-old animals, centronucleation was observed in the hDMDm7 mouse but not in the WT hDMD mouse. In addition, fibrosis was also observed in the 6-month-old hDMDm7 mouse in the diaphragm (asterisk). FIG. 5B shows TA muscle force assessment as measured by normalized specific force following tetanic contraction. FIG. 5B shows that muscle force from 3- and 6-month old hDMDm7 (delCH2) mice decreases over time as the mouse ages compared to the wild-type mouse (hDMD) at 3 and 6 months of age. FIG. 5C shows the loss of force in the TA muscle following repetitive eccentric contractions. The hDMDm7 (delCH2) mouse lost about 55% TA strength at 3-months old and about 65% TA strength at 6-months, as measured by this assay.

FIG. 6A-C presents results for in vivo exon-skipping experiments in which U7_U7 ESE8_2 SD7 SD6 FORWARD OR REVERSE SC rAAV (4E11vg) was delivered by intramuscular injection in the hDMDm7 mouse. 4E11vg were injected in the TA of hDMDm7 mice. One month post-injection, RNA was isolated and RT-PCR were performed. FIG. 6A shows that both untreated wild-type mice (hDMD, lanes 1 and 2) and mice of the mouse model of DMD (hDMDm7, lanes 3 and 4) expressed transcripts for exons 5, 6, 7, 8, 9, and 10 and for exons 5, 6, 7, 8, and 10, as shown in the top two arrows. Mice injected with AAV1.U7 ESE8_2 SD7 SD6 Forward (lanes 5-10) displayed various amounts of exon skipping of exons 7-9 (top box, outlined in green; with less efficient skipping of exon 6) and exons 6-9 (bottom box, outlined I red; with more efficient skipping of exon 6), as demonstrated by the transcripts of exons 5, 6, and 10, and exons 5 and 10. These results are slightly different from the in vitro data shown in FIG. 2A where exon 6 was excluded but exon 9 was present in one of the transcripts following exon skipping. The transcript containing exon 6, but missing exons 7, 8 and/or 9, does not restore the reading frame; however, the transcript containing (1) exons 5, 9, and 10, or (2) exons 5 and 10 restores the reading frame. FIG. 6B-C shows Sanger sequencing of the transcripts confirming expression of transcripts for exons 5, 6, and 10 (FIG. 6B) and exons 5 and 10 (FIG. 6C) following treatment with the antisense constructs.

FIG. 7 shows antisense, U7, and DMD target sequences of the disclosure. The promoter (yellow), smOPT (green), Loop (blue), and 3′UTR region (pink) are highlighted in different colors.

FIG. 8 shows some examples of U7 antisense construct sequences comprising multiple antisense sequences of the disclosure. The promoter (yellow), smOPT (green), Loop (blue), and 3′UTR region (pink) are highlighted in different colors. In each construct, there are three antisense sequences, each with their own promoter, smOPT, Loop, and 3′UTR sequence.

FIG. 9 shows the efficacy of exon skipping following intramuscular injection of three hDMDm7 mice into the left tibialis anterior (LTA) and right tibialis anterior (RTA). Mice were injected with scAAV1.nESE8_SD7_SD6 (TT745-3) designed to skip exons 6, 7, and 8. Results are from RT-PCR conducted on RNA extracted from treated muscle. Bands sequence were confirmed by Sanger sequencing. Transcripts with exon 5, 6, 10 and exons 5, 10 are in-frame and, thus, therapeutic. To conclude, scAAV1.nESE8_SD7_SD6 (TT745-3) is able to skip exons 7, 8 and 9 and partial skipping of exon 6. Transcript that omits or misses exons 6, 7, 8 and 9 is therapeutic as it restores the reading frame of dmd.

FIG. 10A-B show results from hDMDm7 mice receiving intramuscular injections of AAV.U7snRNA (construct TT744-3) into both TA muscles at 7 weeks of age. Physiology experiments were performed to measure TA muscle force after 3 months. FIG. 10A shows results from measures of specific force in healthy control mice(hDMD-Saline), delCH2/hDMDm7-treated mice (delCH2-TT744-3), and delCH2/hDMDm7 untreated mice (delCH2-Saline), respectively (L to R). FIG. 10B shows results from measures of force from consecutive eccentric contractions from the same groups of mice. TT745-3 is able to ameliorate the specific force of the tibialis anterior and ameliorate the resistance to eccentric force of the tibialis anterior, supporting the therapeutic benefit of this vector.

FIG. 11 shows representative images of 3-month-old hDMDm7 (referred to as DelCH2 in the image) mice treated (AAV.U7snRNA (construct TT744-3); see far right panel labeled “DelCH2+TT744-3-2001-LTA-20x”) and untreated (saline; see middle panel labeled “DelCH2+Sal—2306-LTA-20x”). A healthy control mouse (hDMD 1044) is shown in the far left panel for comparison. Dystrophin is stained in red via immunofluorescent staining. Images are at 20× magnification of the LTA. These results show that treatment with the TT744-3 construct introduced dystrophin expression in the tibialis anterior three months post injection.

FIG. 12A-B show quantification of immunofluorescent staining for dystrophin from 20× magnified images of saline on therapeutic injections of mouse LTA and RTA. FIG. 12A shows automated fiber quantification for fibers with more than 50% dystrophin in the treated mouse compared to the untreated mouse (DelCH2). Compared to the WT, the treated mice have about ˜15 dystrophin positive fibers that has more than 50% dystrophin intensity. FIG. 12B shows automated quantification of dystrophin intensity. The intensity of treated mice is overall ˜550 vs untreated mice is overall ˜480, supporting the fact that this construct allow more dystrophin expression post treatment. Altogether, these results show that treatment with the TT744-3 construct introduced dystrophin expression in the tibialis anterior three months post injection.

FIG. 13 shows quantification of percentage of transcripts from hDMDm7/DelCH2 mice injected intramuscularly with saline (left bar) or construct TT744-3 (right bar). Treatment demonstrates 25% of skipping (in blue corresponding to Δ6-9, the therapeutic transcript) and 75% of none skipped transcript (in orange corresponding to rest, the none skipped transcript). Altogether, this is also data supportive of the efficacy of TT744-3 to mediate skipping of exon 6, 7, 8 and 9 which restores the reading frame of dmd allowing dystrophin expression.

FIG. 14 shows total LTA images (10×) from three mice immunofluorescently stained for dystrophin. hDMD LTA dystrophin (far left panel) shows dystrophin staining in a 5-month-old mouse and acts as a positive control. Staining of LTA for dystrophin 5 months post-injection (DelCH2+Saline; middle panel) shows almost no dystrophin expression. Staining of LTA for dystrophin 5 months post-injection (DelCH2+TT744-3; right panel) shows around ˜40% of dystrophin positive fibers. These results show that treatment with TT744-3 restored dystrophin expression in 40% of fibers supporting its therapeutic potential.

### DETAILED DESCRIPTION

The products and methods described herein are used for preventing disease, delaying the progression of disease, and/or treating muscular dystrophies in patients with one or more mutations of the DMD gene. More particularly, the disclosure provides products and methods using a U7snRNA approach to induce skipping of exons 6, 7, 8, and/or 9 of the DMD gene.

In some aspects, the products and methods described herein induce expression of a dystrophin missing the second part of the actin binding domain 1 (ABD1) to treat patients carrying mutations in exons 6, 7, 8 and/or 9 of the DMD gene encoding dystrophin. Such products and methods force expression of a shorter dystrophin protein isoform.

Garcia and collaborators investigated the use of a vector that allows skipping of both exons 6 and 8 at the same time (US 2012/0077860 (Garcia); Bish et al., Mol. Ther. 20(3):580-9, 2012; Vulin et al., Mol. Ther. 20(11):2120-33, 2012; Barbash et al., Gene Therapy 20:274-82, 2013; and Le Guiner et al., Mol. Ther. 22(11): 1923-35; 2014). Such vector allowed for the expression of a shorter dystrophin (possibly the delta CH2 protein) in the GRMD dog model, leading to a skeletal muscle and cardiac improvement.

The disclosure provides new antisense sequences designed to target each of exons 6, 7, and 8 in the human DMD gene in order to treat patients with mutations in exons 6, 7, 8 and/or 9 of the DMD gene encoding dystrophin. In some aspects, the disclosure includes nucleic acids comprising at least two antisense sequences, each antisense sequence preceded by a U7 promoter. In some aspects, the disclosure includes nucleic acids comprising at least three antisense sequences, each antisense sequence preceded by a U7 promoter. In some aspects, the disclosure includes at least three antisense sequences, each antisense sequence preceded by a U7 promoter, and each antisense sequence targeting a different exon, for example, one targeting exon 6, one targeting exon 7, and one targeting exon 8. In some aspects, the disclosure includes multiple copies of constructs comprising at least two or at least three or more antisense sequences. In some aspects, the disclosure includes vectors comprising multiple copies of such constructs, wherein each construct comprises at least two or at least three antisense sequences. In some aspects, the disclosure includes methods and uses of these nucleic acids (and vectors and compositions comprising such nucleic acids) in the treatment, amelioration, or prevention of MD, or in the production of a medicament for use in the treatment of MD.

The disclosure provides antisense targeting sequences (as set out in Table 1 below) and/or combinations of antisense targeting sequences embedded into a modified U7 small nuclear RNA (U7snRNA) (Gorman et al., Proc Natl Acad Sci USA 95, 4929-4934 (1998)). In some aspects, each antisense sequence is preceded by a U7 promoter sequence. This becomes a part of a small nuclear ribosomal protein complex (snRNP) that protects the antisense sequence from degradation and allows for accumulation in the nucleus where splicing occurs (Suter et al., Hum Mol Genet 8, 2415-2423 (1999)). The U7snRNA, which contains internal promoters allowing for continuous transcription of the downstream antisense sequences, in some aspects, is encapsidated into an AAV for widespread tissue delivery. This approach has been shown to be useful in vitro as well as in mouse and dog models of DMD (Wein et al., Nature Medicine 20, 992-1000 (2014); Goyenvalle et al., Hum Mol Genet 21, 2559-2571 (2012); Barbash et al., Gene Ther 20, 274-282 (2013); Bish et al., Mol Ther 20, 580-589 (2012); Vulin et al., Mol Ther 20, 2120-2133 (2012)). Thus, in some aspects, the disclosure provides such U7snRNA for the prevention, treatment, or amelioration of diseases or disorders resulting from mutations of the DMD gene. In some aspects, the DMD gene is the human DMD gene.

TABLE 1
Antisense Sequences.

| No. | Name | Sequence | SEQ ID NO: |
|---|---|---|---|
| 1 | SD6 antisense for U7 | ctcagtaatcttcttacCTATGACTATGGA | 1 |
| | Forward (SD = splice | | |
| | donor) | | |
| 2 | SD6 antisense for U7 | TCCATAGTCATAGgtaagaagattactgag | 2 |
| | Reverse | | |
| 3 | SD7 antisense for U7 | ttaccaacCTTCAGGATCGAGTAGTTT | 3 |
| | Forward | CTCTA | |
| 4 | SD7 antisense for U7 | TAGAGAAACTACTCGATCCTGAAGgt | 4 |
| | Reverse | tggtaa | |
| 5 | ESE7 antisense for U7 | TGTTGAATGCATGTTCCAGTCGTTG | 5 |
| | Forward (ESE = exon | TGTGG | |
| | splice exon) | | |
| 6 | ESE7 antisense for U7 | CCACACAACGACTGGAACATGCATT | 6 |
| | Reverse | CAACA | |
| 7 | ESE8 antisense for U7 | CTTGGAAGAGTGATGTGATGTACA | 7 |
| | Forward | | |
| 8 | ESE8 antisense for U7 | TGTACATCACATCACTCTTCCAAG | 8 |
| | Reverse | | |
| 9 | ESE8_2 antisense for U7 | TGATGTAACTGAAAATGTTCTTCTTT | 9 |
| | Forward | AGTCACTTTAGGTGGCCT | |
| 10 | ESE8_2 antisense for U7 | AGGCCACCTAAAGTGACTAAAGAA | 10 |
| | Reverse | GAACATTTTCAGTTACATCA | |

The U7 can be cloned into two orientations: forward and reverse. When the U7 is in the forward orientation, the forward antisense sequence is used. When the U7 is in the reverse orientation, the reverse antisense sequence is used. Lower case letters in the sequence represent bases of an intronic region. Upper case letters in the sequence represent bases of an exonic region.

The disclosure provides antisense, U7, and target sequences of the DMD gene (as set out in Table 2 below).

TABLE 2
Antisense, U7, and DMD Target Sequences.

| | | SEQ ID |
|---|---|---|
| Name | Sequence | NO: |
| SD6 exonic | TCCATAGTCATAGgtaagaagattactgag | 21 |
| target | | |
| SD6 antisense | ctcagtaatcttcttacCTATGACTATGGA | 1 |
| for U7 Forward | | |
| U7_SD6 forward | taacaacataggagctgtgattggctgttttcagccaatcagcactgactcatttgc | 11 |
| | atagcctttacaagcggtcacaaactcaagaaacgagcggttttaatagtctttta | |
| | gaatattgtttatcgaaccgaataaggaactgtgctttgtgattcacatatcagtgg | |
| | aggggtgtggaaatggcaccttgatctcaccctcatcgaaagtggagttgatgtc | |
| | cttccctggctcgctacagacgcacttccgcaactcagtaatcttcttacCTATG | |
| | ACTATGGAaatttttggagcaggttttctgacttcggtcggaaaacccctccc | |
| | aatttcactggtctacaatgaaagcaaaacagttctcttccccgctccccggtgtgt | |
| | gagaggggctttgatccttctctggtttcctaggaaacgcgtatgtg | |
| U7 promoter | taacaacataggagctgtgattggctgttttcagccaatcagcactgactcatttgc | 30 |
| | atagcctttacaagcggtcacaaactcaagaaacgagcggttttaatagtctttta | |
| | gaatattgtttatcgaaccgaataaggaactgtgctttgtgattcacatatcagtgg | |
| | aggggtgtggaaatggcaccttgatctcaccctcatcgaaagtggagttgatgtc | |
| | cttccctggctcgctacagacgcacttccgcaa | |
| smOPT | aatttttggag | 31 |
| Loop | caggttttctgacttcggtcggaaaacccct | 32 |
| 3′UTR | cccaatttcactggtctacaatgaaagcaaaacagttctcttccccgctccccggt | 33 |
| | gtgtgagaggggctttgatccttctctggtttcctaggaaacgcgtatgtg | |
| SD6 antisense | TCCATAGTCATAGgtaagaagattactgag | 2 |
| for U7 reverse | | |
| U7_SD6 reverse | cacatacgcgtttcctaggaaaccagagaaggatcaaagcccctctcacacac | 12 |
| | cggggagcggggaagagaactgttttgctttcattgtagaccagtgaaattggga | |
| | ggggttttccgaccgaagtcagaaaacctgctccaaaaattTCCATAGTC | |
| | ATAGgtaagaagattactgagttgcggaagtgcgtctgtagcgagccaggga | |
| | aggacatcaactccactttcgatgagggtgagatcaaggtgccatttccacaccc | |
| | ctccactgatatgtgaatcacaaagcacagttccttattcggttcgataaacaatat | |
| | tctaaaagactattaaaaccgctcgtttcttgagtttgtgaccgcttgtaaaggctat | |
| | gcaaatgagtcagtgctgattggctgaaaacagccaatcacagctcctatgttgtt | |
| | a | |
| U7 promoter | ttgcggaagtgcgtctgtagcgagccagggaaggacatcaactccactttcgat | 34 |
| | gagggtgagatcaaggtgccatttccacacccctccactgatatgtgaatcacaa | |
| | agcacagttccttattcggttcgataaacaatattctaaaagactattaaaaccgct | |
| | cgtttcttgagtttgtgaccgcttgtaaaggctatgcaaatgagtcagtgctgattgg | |
| | ctgaaaacagccaatcacagctcctatgttgtta | |
| smOPT | ctccaaaaatt | 35 |
| Loop | aggggttttccgaccgaagtcagaaaacctg | 36 |
| 3′UTR | cacatacgcgtttcctaggaaaccagagaaggatcaaagcccctctcacacac | 37 |
| | cggggagcggggaagagaactgttttgctttcattgtagaccagtgaaattggg | |
| SD7 exonic | TAGAGAAACTACTCGATCCTGAAGgttggtaa | 22 |
| target | | |
| SD7 antisense | ttaccaacCTTCAGGATCGAGTAGTTTCTCTA | 3 |
| for U7 Forward | | |
| U7_SD7 forward | taacaacataggagctgtgattggctgttttcagccaatcagcactgactcatttgc | 13 |
| | atagcctttacaagcggtcacaaactcaagaaacgagcggttttaatagtctttta | |
| | gaatattgtttatcgaaccgaataaggaactgtgctttgtgattcacatatcagtgg | |
| | aggggtgtggaaatggcaccttgatctcaccctcatcgaaagtggagttgatgtc | |
| | cttccctggctcgctacagacgcacttccgcaattaccaacCTTCAGGATC | |
| | GAGTAGTTTCTCTAaatttttggagcaggttttctgacttcggtcggaaaa | |
| | cccctcccaatttcactggtctacaatgaaagcaaaacagttctcttccccgctcc | |
| | ccggtgtgtgagaggggctttgatccttctctggtttcctaggaaacgcgtatgtg | |
| U7 promoter | taacaacataggagctgtgattggctgttttcagccaatcagcactgactcatttgc | 30 |
| | atagcctttacaagcggtcacaaactcaagaaacgagcggttttaatagtctttta | |
| | gaatattgtttatcgaaccgaataaggaactgtgctttgtgattcacatatcagtgg | |
| | aggggtgtggaaatggcaccttgatctcaccctcatcgaaagtggagttgatgtc | |
| | cttccctggctcgctacagacgcacttccgcaa | |
| smOPT | aatttttggag | 31 |
| Loop | caggttttctgacttcggtcggaaaacccct | 32 |
| 3′UTR | cccaatttcactggtctacaatgaaagcaaaacagttctcttccccgctccccggt | 33 |
| | gtgtgagaggggctttgatccttctctggtttcctaggaaacgcgtatgtg | |
| SD7 antisense | TAGAGAAACTACTCGATCCTGAAGgttggtaa | 4 |
| for U7 reverse | | |
| U7_SD7 reverse | cacatacgcgtttcctaggaaaccagagaaggatcaaagcccctctcacacac | 14 |
| | cggggagcggggaagagaactgttttgctttcattgtagaccagtgaaattggga | |
| | ggggttttccgaccgaagtcagaaaacctgctccaaaaattTAGAGAAAC | |
| | TACTCGATCCTGAAGgttggtaattgcggaagtgcgtctgtagcgagcc | |
| | agggaaggacatcaactccactttcgatgagggtgagatcaaggtgccatttcc | |
| | acacccctccactgatatgtgaatcacaaagcacagttccttattcggttcgataa | |
| | acaatattctaaaagactattaaaaccgctcgtttcttgagtttgtgaccgcttgtaa | |
| | aggctatgcaaatgagtcagtgctgattggctgaaaacagccaatcacagctcc | |
| | tatgttgtta | |
| U7 promoter | ttgcggaagtgcgtctgtagcgagccagggaaggacatcaactccactttcgat | 34 |
| | gagggtgagatcaaggtgccatttccacacccctccactgatatgtgaatcacaa | |
| | agcacagttccttattcggttcgataaacaatattctaaaagactattaaaaccgct | |
| | cgtttcttgagtttgtgaccgcttgtaaaggctatgcaaatgagtcagtgctgattgg | |
| | ctgaaaacagccaatcacagctcctatgttgtta | |
| smOPT | ctccaaaaatt | 35 |
| Loop | aggggttttccgaccgaagtcagaaaacctg | 36 |
| 3′UTR | cacatacgcgtttcctaggaaaccagagaaggatcaaagcccctctcacacac | 37 |
| | cggggagcggggaagagaactgttttgctttcattgtagaccagtgaaattgg | |
| ESE7 exonic | CCACACAACGACTGGAACATGCATTCAACA | 23 |
| target | | |
| ESE7 antisense | TGTTGAATGCATGTTCCAGTCGTTGTGTGG | 5 |
| for U7 Forward | | |
| U7_ESE7 | taacaacataggagctgtgattggctgttttcagccaatcagcactgactcatttgc | 15 |
| forward | atagcctttacaagcggtcacaaactcaagaaacgagcggttttaatagtctttta | |
| | gaatattgtttatcgaaccgaataaggaactgtgctttgtgattcacatatcagtgg | |
| | aggggtgtggaaatggcaccttgatctcaccctcatcgaaagtggagttgatgtc | |
| | cttccctggctcgctacagacgcacttccgcaaTGTTGAATGCATGTT | |
| | CCAGTCGTTGTGTGGaatttttggagcaggttttctgacttcggtcggaa | |
| | aacccctcccaatttcactggtctacaatgaaagcaaaacagttctcttccccgct | |
| | ccccggtgtgtgagaggggctttgatccttctctggtttcctaggaaacgcgtatgt | |
| | g | |
| U7 promoter | taacaacataggagctgtgattggctgttttcagccaatcagcactgactcatttgc | 30 |
| | atagcctttacaagcggtcacaaactcaagaaacgagcggttttaatagtctttta | |
| | gaatattgtttatcgaaccgaataaggaactgtgctttgtgattcacatatcagtgg | |
| | aggggtgtggaaatggcaccttgatctcaccctcatcgaaagtggagttgatgtc | |
| | cttccctggctcgctacagacgcacttccgcaa | |
| smOPT | aatttttggag | 31 |
| Loop | caggttttctgacttcggtcggaaaacccct | 32 |
| 3′UTR | cccaatttcactggtctacaatgaaagcaaaacagttctcttccccgctccccggt | 33 |
| | gtgtgagaggggctttgatccttctctggtttcctaggaaacgcgtatgtg | |
| ESE7 antisense | CCACACAACGACTGGAACATGCATTCAACA | 6 |
| for U7 reverse | | |
| U7_ESE7 | cacatacgcgtttcctaggaaaccagagaaggatcaaagcccctctcacacac | 16 |
| reverse | cggggagcggggaagagaactgttttgctttcattgtagaccagtgaaattggga | |
| | ggggttttccgaccgaagtcagaaaacctgctccaaaaattCCACACAAC | |
| | GACTGGAACATGCATTCAACAttgcggaagtgcgtctgtagcgag | |
| | ccagggaaggacatcaactccactttcgatgagggtgagatcaaggtgccattt | |
| | ccacacccctccactgatatgtgaatcacaaagcacagttccttattcggttcgat | |
| | aaacaatattctaaaagactattaaaaccgctcgtttcttgagtttgtgaccgcttgt | |
| | aaaggctatgcaaatgagtcagtgctgattggctgaaaacagccaatcacagct | |
| | cctatgttgtta | |
| U7 promoter | ttgcggaagtgcgtctgtagcgagccagggaaggacatcaactccactttcgat | 34 |
| | gagggtgagatcaaggtgccatttccacacccctccactgatatgtgaatcacaa | |
| | agcacagttccttattcggttcgataaacaatattctaaaagactattaaaaccgct | |
| | cgtttcttgagtttgtgaccgcttgtaaaggctatgcaaatgagtcagtgctgattgg | |
| | ctgaaaacagccaatcacagctcctatgttgtta | |
| smOPT | ctccaaaaatt | 35 |
| Loop | aggggttttccgaccgaagtcagaaaacctg | 36 |
| 3′UTR | cacatacgcgtttcctaggaaaccagagaaggatcaaagcccctctcacacac | 37 |
| | cggggagcggggaagagaactgttttgctttcattgtagaccagtgaaattggg | |
| ESE8 exonic | TGTACATCACATCACTCTTCCAAG | 24 |
| target | | |
| ESE8 antisense | CTTGGAAGAGTGATGTGATGTACA | 7 |
| for U7 Forward | | |
| U7_ESE8 | taacaacataggagctgtgattggctgttttcagccaatcagcactgactcatttgc | 17 |
| forward | atagcctttacaagcggtcacaaactcaagaaacgagcggttttaatagtctttta | |
| | gaatattgtttatcgaaccgaataaggaactgtgctttgtgattcacatatcagtgg | |
| | aggggtgtggaaatggcaccttgatctcaccctcatcgaaagtggagttgatgtc | |
| | cttccctggctcgctacagacgcacttccgcaaCTTGGAAGAGTGATG | |
| | TGATGTACAaatttttggagcaggttttctgacttcggtcggaaaacccctcc | |
| | caatttcactggtctacaatgaaagcaaaacagttctcttccccgctccccggtgt | |
| | gtgagaggggctttgatccttctctggtttcctaggaaacgcgtatgtg | |
| U7 promoter | taacaacataggagctgtgattggctgttttcagccaatcagcactgactcatttgc | 30 |
| | atagcctttacaagcggtcacaaactcaagaaacgagcggttttaatagtctttta | |
| | gaatattgtttatcgaaccgaataaggaactgtgctttgtgattcacatatcagtgg | |
| | aggggtgtggaaatggcaccttgatctcaccctcatcgaaagtggagttgatgtc | |
| | cttccctggctcgctacagacgcacttccgcaa | |
| smOPT | aatttttggag | 31 |
| Loop | caggttttctgacttcggtcggaaaacccct | 32 |
| 3′UTR | cccaatttcactggtctacaatgaaagcaaaacagttctcttccccgctccccggt | 33 |
| | gtgtgagaggggctttgatccttctctggtttcctaggaaacgcgtatgtg | |
| ESE8 antisense | TGTACATCACATCACTCTTCCAAG | 8 |
| for U7 reverse | | |
| U7_ESE8 | cacatacgcgtttcctaggaaaccagagaaggatcaaagcccctctcacacac | 18 |
| reverse | cggggagcggggaagagaactgttttgctttcattgtagaccagtgaaattggga | |
| | ggggttttccgaccgaagtcagaaaacctgctccaaaaattTGTACATCA | |
| | CATCACTCTTCCAAGttgcggaagtgcgtctgtagcgagccagggaa | |
| | ggacatcaactccactttcgatgagggtgagatcaaggtgccatttccacacccc | |
| | tccactgatatgtgaatcacaaagcacagttccttattcggttcgataaacaatatt | |
| | ctaaaagactattaaaaccgctcgtttcttgagtttgtgaccgcttgtaaaggctatg | |
| | caaatgagtcagtgctgattggctgaaaacagccaatcacagctcctatgttgtta | |
| U7 promoter | ttgcggaagtgcgtctgtagcgagccagggaaggacatcaactccactttcgat | 34 |
| | gagggtgagatcaaggtgccatttccacacccctccactgatatgtgaatcacaa | |
| | agcacagttccttattcggttcgataaacaatattctaaaagactattaaaaccgct | |
| | cgtttcttgagtttgtgaccgcttgtaaaggctatgcaaatgagtcagtgctgattgg | |
| | ctgaaaacagccaatcacagctcctatgttgtta | |
| smOPT | ctccaaaaatt | 35 |
| Loop | ggggttttccgaccgaagtcagaaaacctg | 36 |
| 3′UTR | cacatacgcgtttcctaggaaaccagagaaggatcaaagcccctctcacacac | 37 |
| | cggggagcggggaagagaactgttttgctttcattgtagaccagtgaaattggg | |
| ESE8 2 exonic | AGGCCACCTAAAGTGACTAAAGAAGAACATTTTCAGTT | 25 |
| target | ACATCA | |
| ESE8_2 | TGATGTAACTGAAAATGTTCTTCTTTAGTCACTTTAGGT | 9 |
| antisense for U7 | GGCCT | |
| Forward | | |
| U7_ESE8_2 | taacaacataggagctgtgattggctgttttcagccaatcagcactgactcatttgc | 19 |
| forward | atagcctttacaagcggtcacaaactcaagaaacgagcggttttaatagtctttta | |
| | gaatattgtttatcgaaccgaataaggaactgtgctttgtgattcacatatcagtgg | |
| | aggggtgtggaaatggcaccttgatctcaccctcatcgaaagtggagttgatgtc | |
| | cttccctggctcgctacagacgcacttccgcaaTGATGTAACTGAAAAT | |
| | GTTCTTCTTTAGTCACTTTAGGTGGCCTaatttttggagcaggtt | |
| | ttctgacttcggtcggaaaacccctcccaatttcactggtctacaatgaaagcaaa | |
| | acagttctcttccccgctccccggtgtgtgagaggggctttgatccttctctggtttcc | |
| | taggaaacgcgtatgtg | |
| U7 promoter | taacaacataggagctgtgattggctgttttcagccaatcagcactgactcatttgc | 30 |
| | atagcctttacaagcggtcacaaactcaagaaacgagcggttttaatagtctttta | |
| | gaatattgtttatcgaaccgaataaggaactgtgctttgtgattcacatatcagtgg | |
| | aggggtgtggaaatggcaccttgatctcaccctcatcgaaagtggagttgatgtc | |
| | cttccctggctcgctacagacgcacttccgcaa | |
| smOPT | aatttttggag | 31 |
| Loop | caggttttctgacttcggtcggaaaacccct | 32 |
| 3′UTR | cccaatttcactggtctacaatgaaagcaaaacagttctcttccccgctccccggt | 33 |
| | gtgtgagaggggctttgatccttctctggtttcctaggaaacgcgtatgtg | |
| ESE8_2 | AGGCCACCTAAAGTGACTAAAGAAGAACATTTTCAGTT | 10 |
| antisense for U7 | ACATCA | |
| reverse | | |
| U7_ESE8_2 | cacatacgcgtttcctaggaaaccagagaaggatcaaagcccctctcacacac | 20 |
| reverse | cggggagcggggaagagaactgttttgctttcattgtagaccagtgaaattggga | |
| | ggggttttccgaccgaagtcagaaaacctgctccaaaaattAGGCCACCT | |
| | AAAGTGACTAAAGAAGAACATTTTCAGTTACATCAttgcgg | |
| | aagtgcgtctgtagcgagccagggaaggacatcaactccactttcgatgagggt | |
| | gagatcaaggtgccatttccacacccctccactgatatgtgaatcacaaagcac | |
| | agttccttattcggttcgataaacaatattctaaaagactattaaaaccgctcgtttct | |
| | tgagtttgtgaccgcttgtaaaggctatgcaaatgagtcagtgctgattggctgaa | |
| | aacagccaatcacagctcctatgttgtta | |
| U7 promoter | ttgcggaagtgcgtctgtagcgagccagggaaggacatcaactccactttcgat | 34 |
| | gagggtgagatcaaggtgccatttccacacccctccactgatatgtgaatcacaa | |
| | agcacagttccttattcggttcgataaacaatattctaaaagactattaaaaccgct | |
| | cgtttcttgagtttgtgaccgcttgtaaaggctatgcaaatgagtcagtgctgattgg | |
| | ctgaaaacagccaatcacagctcctatgttgtta | |
| smOPT | ctccaaaaatt | 35 |
| Loop | aggggttttccgaccgaagtcagaaaacctg | 36 |
| 3′UTR | cacatacgcgtttcctaggaaaccagagaaggatcaaagcccctctcacacac | 37 |
| | cggggagcggggaagagaactgttttgctttcattgtagaccagtgaaattggg | |

The disclosure provides nucleic acids comprising combinations of antisense targeting sequences (as set out in Table 3 below). In some aspects, the sequences are embedded into a modified U7 small nuclear RNA (U7snRNA). Such constructs as set out in Table 3 below are not limiting as to nucleic acids comprising other possible combinations of the antisense sequences set out in any of SEQ ID NOs: 1-20, or variants thereof, or antisense sequences targeting any of the nucleotide sequences set forth in any of SEQ ID NOs: 21-25.

TABLE 3
U7 Sequences Comprising Multiple Antisense Sequences.

| Name | Sequence | SEQ ID NO: |
|---|---|---|
| U7_ESE8_2_ESE7_ | taacaacataggagctgtgattggctgttttcagccaatcagcactgactcattt | 26 |
| SD6_forward | gcatagcctttacaagcggtcacaaactcaagaaacgagcggttttaatagt | |
| | cttttagaatattgtttatcgaaccgaataaggaactgtgctttgtgattcacatat | |
| | cagtggaggggtgtggaaatggcaccttgatctcaccctcatcgaaagtgg | |
| | agttgatgtccttccctggctcgctacagacgcacttccgcaaTGATGTA | |
| | ACTGAAAATGTTCTTCTTTAGTCACTTTAGGTGGCCT | |
| | aatttttggagcaggttttctgacttcggtcggaaaacccctcccaatttcactg | |
| | gtctacaatgaaagcaaaacagttctcttccccgctccccggtgtgtgagag | |
| | gggctttgatccttctctggtttcctaggaaacgcgtatgtggggccctaacaa | |
| | cataggagctgtgattggctgttttcagccaatcagcactgactcatttgcatag | |
| | cctttacaagcggtcacaaactcaagaaacgagcggttttaatagtcttttag | |
| | aatattgtttatcgaaccgaataaggaactgtgctttgtgattcacatatcagtg | |
| | gaggggtgtggaaatggcaccttgatctcaccctcatcgaaagtggagttga | |
| | tgtccttccctggctcgctacagacgcacttccgcaaTGTTGAATGCA | |
| | TGTTCCAGTCGTTGTGTGGaatttttggagcaggttttctgacttcg | |
| | gtcggaaaacccctcccaatttcactggtctacaatgaaagcaaaacagttc | |
| | tcttccccgctccccggtgtgtgagaggggctttgatccttctctggtttcctagg | |
| | aaacgcgtatgtgatcgattaacaacataggagctgtgattggctgttttcagc | |
| | caatcagcactgactcatttgcatagcctttacaagcggtcacaaactcaag | |
| | aaacgagcggttttaatagtcttttagaatattgtttatcgaaccgaataagga | |
| | actgtgctttgtgattcacatatcagtggaggggtgtggaaatggcaccttgat | |
| | ctcaccctcatcgaaagtggagttgatgtccttccctggctcgctacagacgc | |
| | acttccgcaactcagtaatcttcttacCTATGACTATGGAaatttttgga | |
| | gcaggttttctgacttcggtcggaaaacccctcccaatttcactggtctacaat | |
| | gaaagcaaaacagttctcttccccgctccccggtgtgtgagaggggctttgat | |
| | ccttctctggtttcctaggaaacgcgtatgtgctcgagaggggctttgatccttct | |
| | ctggtttcctaggaaacgcgtatgtg | |
| U7_ESE8_2_SD7_ | taacaacataggagctgtgattggctgttttcagccaatcagcactgactcattt | 27 |
| SD6_forward | gcatagcctttacaagcggtcacaaactcaagaaacgagcggttttaatagt | |
| | cttttagaatattgtttatcgaaccgaataaggaactgtgctttgtgattcacatat | |
| | cagtggaggggtgtggaaatggcaccttgatctcaccctcatcgaaagtgg | |
| | agttgatgtccttccctggctcgctacagacgcacttccgcaaTGATGTA | |
| | ACTGAAAATGTTCTTCTTTAGTCACTTTAGGTGGCCT | |
| | aatttttggagcaggttttctgacttcggtcggaaaacccctcccaatttcactg | |
| | gtctacaatgaaagcaaaacagttctcttccccgctccccggtgtgtgagag | |
| | gggctttgatccttctctggtttcctaggaaacgcgtatgtggggccctaacaa | |
| | cataggagctgtgattggctgttttcagccaatcagcactgactcatttgcatag | |
| | cctttacaagcggtcacaaactcaagaaacgagcggttttaatagtcttttag | |
| | aatattgtttatcgaaccgaataaggaactgtgctttgtgattcacatatcagtg | |
| | gaggggtgtggaaatggcaccttgatctcaccctcatcgaaagtggagttga | |
| | tgtccttccctggctcgctacagacgcacttccgcaattaccaacCTTCAG | |
| | GATCGAGTAGTTTCTCTAaatttttggagcaggttttctgacttcggt | |
| | cggaaaacccctcccaatttcactggtctacaatgaaagcaaaacagttctc | |
| | ttccccgctccccggtgtgtgagaggggctttgatccttctctggtttcctagga | |
| | aacgcgtatgtgatcgattaacaacataggagctgtgattggctgttttcagcc | |
| | aatcagcactgactcatttgcatagcctttacaagcggtcacaaactcaaga | |
| | aacgagcggttttaatagtcttttagaatattgtttatcgaaccgaataaggaac | |
| | tgtgctttgtgattcacatatcagtggaggggtgtggaaatggcaccttgatctc | |
| | accctcatcgaaagtggagttgatgtccttccctggctcgctacagacgcactt | |
| | ccgcaactcagtaatcttcttacCTATGACTATGGAaatttttggagca | |
| | ggttttctgacttcggtcggaaaacccctcccaatttcactggtctacaatgaa | |
| | agcaaaacagttctcttccccgctccccggtgtgtgagaggggctttgatcctt | |
| | ctctggtttcctaggaaacgcgtatgtgctcgagaggggctttgatccttctctg | |
| | gtttcctaggaaacgcgtatgtg | |
| U7_ESE8_2_ESE7_ | cacatacgcgtttcctaggaaaccagagaaggatcaaagcccctctcgag | 28 |
| SD6_reverse | cacatacgcgtttcctaggaaaccagagaaggatcaaagcccctctcacac | |
| | accggggagcggggaagagaactgttttgctttcattgtagaccagtgaaatt | |
| | gggaggggttttccgaccgaagtcagaaaacctgctccaaaaattTCCA | |
| | TAGTCATAGgtaagaagattactgagttgcggaagtgcgtctgtagcg | |
| | agccagggAaggacatcaactccactttcgatgagggtgagatcaaggtg | |
| | ccatttccacacccctccactgatatgtgaatcacaaagcacagttccttattc | |
| | ggttcgataaacaatattctaaaagactattaaaaccgctcgtttcttgagtttgt | |
| | gaccgcttgtaaaggctatgcaaatgagtcagtgctgattggctgaaaacag | |
| | ccaatcacagctcctatgttgttaatcgatcacatacgcgtttcctaggaaacc | |
| | agagaaggatcaaagcccctctcacacaccggggagcggggaagagaa | |
| | ctgttttgctttcattgtagaccagtgaaattgggaggggttttccgaccgaagt | |
| | cagaaaacctgctccaaaaattCCACACAACGACTGGAACAT | |
| | GCATTCAACAttgcggaagtgcgtctgtagcgagccagggaaggac | |
| | atcaactccactttcgatgagggtgagatcaaggtgccatttccacacccctc | |
| | cactgatatgtgaatcacaaagcacagttccttattcggttcgataaacaatat | |
| | tctaaaagactattaaaaccgctcgtttcttgagtttgtgaccgcttgtaaaggc | |
| | tatgcaaatgagtcagtgctgattggctgaaaacagccaatcacagctcctat | |
| | gttgttagggccccacatacgcgtttcctaggaaaccagagaaggatcaaa | |
| | gcccctctcacacaccggggagcggggaagagaactgttttgctttcattgta | |
| | gaccagtgaaattgggaggggttttccgaccgaagtcagaaaacctgctcc | |
| | aaaaattAGGCCACCTAAAGTGACTAAAGAAGAACATT | |
| | TTCAGTTACATCAttgcggaagtgcgtctgtagcgagccagggaag | |
| | gacatcaactccactttcgatgagggtgagatcaaggtgccatttccacacc | |
| | cctccactgatatgtgaatcacaaagcacagttccttattcggttcgataaaca | |
| | atattctaaaagactattaaaaccgctcgtttcttgagtttgtgaccgcttgtaaa | |
| | ggctatgcaaatgagtcagtgctgattggctgaaaacagccaatcacagctc | |
| | ctatgttgtta | |
| U7_ESE8_2_SD7_ | cacatacgcgtttcctaggaaaccagagaaggatcaaagcccctctcgag | 29 |
| SD6_reverse | cacatacgcgtttcctaggaaaccagagaaggatcaaagcccctctcacac | |
| | accggggagcggggaagagaactgttttgctttcattgtagaccagtgaaatt | |
| | gggaggggttttccgaccgaagtcagaaaacctgctccaaaaattTCCA | |
| | TAGTCATAGgtaagaagattactgagttgcggaagtgcgtctgtagcg | |
| | agccagggAaggacatcaactccactttcgatgagggtgagatcaaggtg | |
| | ccatttccacacccctccactgatatgtgaatcacaaagcacagttccttattc | |
| | ggttcgataaacaatattctaaaagactattaaaaccgctcgtttcttgagtttgt | |
| | gaccgcttgtaaaggctatgcaaatgagtcagtgctgattggctgaaaacag | |
| | ccaatcacagctcctatgttgttaatcgatcacatacgcgtttcctaggaaacc | |
| | agagaaggatcaaagcccctctcacacaccggggagcggggaagagaa | |
| | ctgttttgctttcattgtagaccagtgaaattgggaggggttttccgaccgaagt | |
| | cagaaaacctgctccaaaaattTAGAGAAACTACTCGATCCT | |
| | GAAGgttggtaattgcggaagtgcgtctgtagcgagccagggaaggaca | |
| | tcaactccactttcgatgagggtgagatcaaggtgccatttccacacccctcc | |
| | actgatatgtgaatcacaaagcacagttccttattcggttcgataaacaatatt | |
| | ctaaaagactattaaaaccgctcgtttcttgagtttgtgaccgcttgtaaaggct | |
| | atgcaaatgagtcagtgctgattggctgaaaacagccaatcacagctcctat | |
| | gttgttagggccccacatacgcgtttcctaggaaaccagagaaggatcaaa | |
| | gcccctctcacacaccggggagcggggaagagaactgttttgctttcattgta | |
| | gaccagtgaaattgggaggggttttccgaccgaagtcagaaaacctgctcc | |
| | aaaaattAGGCCACCTAAAGTGACTAAAGAAGAACATT | |
| | TTCAGTTACATCAttgcggaagtgcgtctgtagcgagccagggaag | |
| | gacatcaactccactttcgatgagggtgagatcaaggtgccatttccacacc | |
| | cctccactgatatgtgaatcacaaagcacagttccttattcggttcgataaaca | |
| | atattctaaaagactattaaaaccgctcgtttcttgagtttgtgaccgcttgtaaa | |
| | ggctatgcaaatgagtcagtgctgattggctgaaaacagccaatcacagctc | |
| | ctatgttgtta | |

The disclosure thus provides a nucleic acid comprising any of the sequences set out in Tables 1-3. More specifically, the disclosure provides a nucleic acid comprising a nucleotide sequence comprising at least 70% or 80% identity to the sequence set forth in any one of SEQ ID NOs: 1-20 and 26-29; a nucleotide sequence complementary to the nucleotide sequence comprising at least 70% or 80% identity to the sequence set forth in any one of SEQ ID NOs: 1-20 and 26-29; a nucleotide sequence comprising the sequence set forth in any one of SEQ ID NOs: 1-20 and 26-29; a nucleotide sequence complementary to the nucleotide sequence comprising the sequence set forth in any one of SEQ ID NOs: 1-20 and 26-29; or a nucleotide sequence which binds to the sequence set forth in any one of SEQ ID NOs: 21-25.

In some aspects, the disclosure provides a nucleic acid comprising a combination of at least two, at least three, at least four, at least five, at least six, at least seven, at least eight, at least nine, or at least ten or more nucleotide sequences of: (a) a nucleotide sequence comprising at least 70% or 80% identity to the sequence set forth in any one of SEQ ID NOs: 1-20 and 26-29; (b) a nucleotide sequence complementary to the nucleotide sequence comprising at least 70% or 80% identity to the sequence set forth in any one of SEQ ID NOs: 1-20 and 26-29; (c) a nucleotide sequence comprising the sequence set forth in any one of SEQ ID NOs: 1-20 and 26-29; (d) a nucleotide sequence complementary to the nucleotide sequence comprising the sequence set forth in any one of SEQ ID NOs: 1-20 and 26-29; or (e) a nucleotide sequence which binds to the sequence set forth in any one of SEQ ID NOs: 21-25.

In some aspects, the disclosure includes a nucleic acid comprising a nucleotide sequence that has at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% identity to the sequence set forth in set forth in any one of SEQ ID NOs: 1-20 and 26-29.

In some aspects, the disclosure includes a nucleic acid comprising a nucleotide sequence that is complementary to a nucleotide sequence that has at least about 70%, 75%, 80%, 81%, 82%, 83%, 84%, 85%, 86%, 87%, 88%, 89%, 90%, 91%, 92%, 93%, 94%, 95%, 96%, 97%, 98%, 99% or 100% identity to a sequence set forth in any one of SEQ ID NOs: 1-20 and 26-29.

In some aspects, the disclosure includes a nucleotide sequence comprising the sequence set forth in any one of SEQ ID NOs: 1-20 and 26-29. In some aspects, the disclosure includes a nucleotide sequence complementary to the nucleotide sequence comprising the sequence set forth in any one of SEQ ID NOs: 1-20 and 26-29. In some aspects, the disclosure includes a nucleic acid comprising a nucleotide sequence which binds to the sequence set forth in any one of SEQ ID NOs: 21-25.

The disclosure thus provides a nucleic acid comprising a nucleotide sequence comprising at least 80% identity to the sequence set forth in any one of SEQ ID NOs: 1-20 and 26-29; a nucleotide sequence complementary to the nucleotide sequence comprising at least 80% identity to the sequence set forth in any one of SEQ ID NOs: 1-20 and 26-29; a nucleotide sequence comprising the sequence set forth in any one of SEQ ID NOs: 1-20 and 26-29; a nucleotide sequence complementary to the nucleotide sequence comprising the sequence set forth in any one of SEQ ID NOs: 1-20 and 26-29; and a nucleotide sequence which binds to the sequence set forth in any one of SEQ ID NOs: 21-25.

In some aspects, the nucleic acid comprises a combination of at least two nucleotide sequences, wherein the combination comprises a nucleotide sequence that targets the human DMD gene at exon 6 and a nucleotide sequence that targets the human DMD gene at exon 7, a nucleotide sequence that targets the human DMD gene at exon 6 and a nucleotide sequence that targets the human DMD gene at exon 8, and a nucleotide sequence that targets the human DMD gene at exon 7 and a nucleotide sequence that targets the human DMD gene at exon 8. In some aspects, the nucleic acid comprises a combination of at least three nucleotide sequences, wherein the combination comprises a nucleotide sequence that targets the human DMD gene at exon 6, a nucleotide sequence that targets the human DMD gene at exon 7, and a nucleotide sequence that targets the human DMD gene at exon 8. In some further aspects, the nucleic acid comprises a combination of at least four nucleotide sequences, at least five nucleotide sequences, at least six nucleotide sequences, at least seven nucleotide sequences, at least eight nucleotide sequences, at least nine nucleotide sequences, or at least ten or more nucleotide sequences that target the human DMD gene at exon 6, 7, and/or 8.

In some aspects such nucleotide sequences include any combination of one or more of sequences comprising at least 80% identity to the sequence set forth in any one of SEQ ID NOs: 1-20 and 26-29; a nucleotide sequence complementary to the nucleotide sequence comprising at least 70% or 80% identity to the sequence set forth in any one of SEQ ID NOS: 1-20 and 26-29; a nucleotide sequence comprising the sequence set forth in any one of SEQ ID NOs: 1-20 and 26-29; a nucleotide sequence complementary to the nucleotide sequence comprising the sequence set forth in any one of SEQ ID NOs: 1-20 and 26-29; and a nucleotide sequence which binds to the sequence set forth in any one of SEQ ID NOs: 21-25.

In some aspects, such combination of nucleotide sequences comprises a nucleotide sequence comprising at least 80% identity to the sequence set forth in any one of SEQ ID NOs: 26-29; a nucleotide sequence complementary to the nucleotide sequence comprising at least 70% or 80% identity to the sequence set forth in any one of SEQ ID NOs: 26-29; a nucleotide sequence comprising the sequence set forth in any one of SEQ ID NOs: 26-29; a nucleotide sequence complementary to the nucleotide sequence comprising the sequence set forth in any one of SEQ ID NOs: 26-29.

In some aspects, the disclosure includes such nucleic acid under the control of a U7 promoter. In some aspects, the disclosure includes the delivery of such nucleic acid in a U7 small nuclear RNA (snRNA). In some aspects, the U7 promoter comprises a nucleotide sequence comprising at least 70%, 80%, or 90% identity to the sequence set forth in SEQ ID NO: 30 or 34.

In some aspects, the disclosure provides small nuclear ribonucleic acids (snRNAs), also commonly referred to as U-RNAs, to affect DMD expression. snRNAs are a class of small RNA molecules that are found within the splicing speckles and Cajal bodies of the cell nucleus in eukaryotic cells. Small nuclear RNAs are associated with a set of specific proteins, and the complexes are referred to as small nuclear ribonucleoproteins (snRNP, often pronounced “snurps”). Each snRNP particle is composed of a snRNA component and several snRNP-specific proteins (including Sm proteins, a family of nuclear proteins). The snRNAs, along with their associated proteins, form ribonucleoprotein complexes (snRNPs), which bind to specific sequences on the pre-mRNA substrate. They are transcribed by either RNA polymerase II or RNA polymerase III. snRNAs are often divided into two classes based upon both common sequence features and associated protein factors, such as the RNA-binding LSm proteins. The first class, known as Sm-class snRNA, consists of U1, U2, U4, U4atac, U5, U7, U11, and U12. Sm-class snRNA are transcribed by RNA polymerase II. The second class, known as Lsm-class snRNA, consists of U6 and U6atac. Lsm-class snRNAs are transcribed by RNA polymerase Ill and never leave the nucleus, in contrast to Sm-class snRNA.

In some aspects, the disclosure provides U7 snRNA molecules to interfere with DMD gene expression. U7 snRNA is normally involved in histone pre-mRNA 3′ end processing but, in some aspects, is converted into a versatile tool for splicing modulation or as antisense RNA that is continuously expressed in cells [Goyenvalle et al., Science 306(5702): 1796-9 (2004)]. Moreover, when embedded into a gene therapy vector, these small RNAs can be permanently expressed inside the target cell after a single injection [Levy et al., Eur. J. Hum. Genet. 18(9): 969-70 (2010); Wein et al., Hum. Mutat. 31(2): 136-42, (2010); Wein et al., Nat. Med. 20(9): 992-1000 (2014)]. The potential of U7snRNA systems in neuromuscular disorders using an AAV approach has been investigated in vivo (AAV.U7) [Levy et al., Eur. J. Hum. Genet. 18(9): 969-70 (2010); Wein et al., Hum. Mutat. 31(2): 136-42 (2010); Wein et al., Nat. Med. 20(9): 992-1000 (2014)]. A single injection of this AAV9.U7, targeting the defective RNA of a mouse model of Duchenne muscular dystrophy, results in long term correction of the disease in every muscle, including heart and diaphragm. The ability to target the heart is important since DM1 patients display cardiac abnormalities.

In some aspects, DNA encoding the U7 snRNA gene comprising the DMD inhibitory nucleic acid is delivered in a vector. However, in some other aspects, such DNA is not delivered in an AAV or other vector. The disclosure therefore includes other means of delivering the DNA encoding the antisense constructs described herein. In some aspects, such delivery includes, but is not limited to delivery via liposomes, nanoparticles, or chemical transfection. Chemical transfection introduces DNA by calcium phosphate, lipid, or protein complexes.

Thus, the antisense sequences of the disclosure, in some aspects, are carried by a snRNA and delivered, in some aspects, using viral vectors, such as adeno-associated virus (AAV) or recombinant AAV (rAAV). An advantage of this approach is that the antisense sequence is embedded into a small nuclear ribonucleoprotein (snRNP) complex, thereby protecting it from degradation and causing accumulation in the nucleus where splicing occurs. Moreover, when embedded into a gene therapy vector, these small RNAs can be permanently expressed inside the target cell after a single injection. AAV is a small virus that naturally infects humans without causing any known disease. The virus induces a very mild immune response and can infect both dividing and quiescent cells. Modified versions used for gene therapy persist in an episomal state without integrating into the genome of the host cell. Therefore, AAV vectors have an excellent safety profile, making them very attractive for gene therapy

The disclosure, in some aspects, therefore utilizes AAV to deliver inhibitory U7snRNA to deliver a DMD antisense sequence, which binds to key exon definition elements in the pre-mRNA, inhibiting the recognition of a specific exon by the spliceosome, leading to exclusion of the target exon from the mature RNA, resulting in the expression of dystrophin. U7 snRNA is normally involved in histone pre-mRNA 3′ end processing but, in some aspects, is converted into a versatile tool for splicing modulation or as antisense RNA that is continuously expressed in cells [Goyenvalle et al., Science 306(5702): 1796-9 (2004)]. Moreover, when embedded into a gene therapy vector, these small RNAs can be permanently expressed inside the target cell after a single injection [Levy et al., Eur. J. Hum. Genet. 18(9): 969-70 (2010); Wein et al., Hum. Mutat. 31(2): 136-42, (2010); Wein et al., Nat. Med. 20(9): 992-1000 (2014)]. The potential of U7snRNA systems in neuromuscular disorders using an AAV approach has been investigated in vivo (AAV.U7) [Levy et al., Eur. J. Hum. Genet. 18(9): 969-70 (2010); Wein et al., Hum. Mutat. 31(2): 136-42 (2010); Wein et al., Nat. Med. 20(9): 992-1000 (2014)]. A single injection of this AAV9.U7, targeting the defective RNA of a mouse model of Duchenne muscular dystrophy, results in long term correction of the disease in every muscle, including heart and diaphragm. The ability to target the heart is really important since DM1 patients display cardiac abnormalities.

The disclosure therefore includes the production and administration of an AAV vector comprising U7 snRNA for the delivery of DMD antisense sequences, such as a nucleotide sequence or a combination of at least two nucleotide sequences selected from the group consisting of:
(a) a nucleotide sequence comprising at least 80% identity to the sequence set forth in any one of SEQ ID NOs: 1-20 and 26-29;
(b) a nucleotide sequence complementary to the nucleotide sequence comprising at least 80% identity to the sequence set forth in any one of SEQ ID NOs: 1-20 and 26-29;
(c) a nucleotide sequence comprising the sequence set forth in any one of SEQ ID NOS: 1-20 and 26-29;
(d) a nucleotide sequence complementary to the nucleotide sequence comprising the sequence set forth in any one of SEQ ID NOs: 1-20 and 26-29; and
(e) a nucleotide sequence which binds to the sequence set forth in any one of SEQ ID NOs: 21-25.

The disclosure also includes the production and administration of an AAV vector comprising U7 snRNA for the delivery of DMD antisense sequences, such as a nucleotide sequence or a combination of at least two, at least three, at least four, at least five, at least six, at least seven, at least eight, at least nine, or at least ten or more nucleotide sequences selected from the group consisting of:
(a) a nucleotide sequence comprising at least 80% identity to the sequence set forth in any one of SEQ ID NOs: 1-20 and 26-29;
(b) a nucleotide sequence complementary to the nucleotide sequence comprising at least 80% identity to the sequence set forth in any one of SEQ ID NOs: 1-20 and 26-29;
(c) a nucleotide sequence comprising the sequence set forth in any one of SEQ ID NOs: 1-20 and 26-29;
(d) a nucleotide sequence complementary to the nucleotide sequence comprising the sequence set forth in any one of SEQ ID NOs: 1-20 and 26-29; and
(e) a nucleotide sequence which binds to the sequence set forth in any one of SEQ ID NOs: 21-25.

In some aspects, the disclosure provides one or more copies of the various antisense sequences described herein combined into a single vector. In some aspects, the disclosure provides one or more copies of the various antisense sequences described herein or a combination of two or more, three or more, four or more, five or more, six or more, seven or more, eight or more, nine or more, and ten or more of the various antisense sequences combined into a single vector. Thus, the disclosure includes a vector or vectors comprising a nucleic acid of the disclosure or a combination of nucleic acids of the disclosure. Such vectors include, for example, viral vectors, such as adeno-associated virus (AAV), adenovirus, retrovirus, lentivirus, equine-associated virus, alphavirus, pox virus, herpes virus, herpes simplex virus, polio virus, sindbis virus, vaccinia virus or a synthetic virus, e.g., a chimeric virus, mosaic virus, or pseudotyped virus, and/or a virus that contains a foreign protein, synthetic polymer, nanoparticle, or small molecule, to deliver one or more of the nucleic acids disclosed herein. In some aspects, the viral vector is an AAV. Thus, in some aspects of the disclosure, U7snRNA is delivered via a viral vector, such as AAV.

In some aspects, the AAV lacks rep and cap genes. In some aspects, the AAV is a recombinant AAV (rAAV). In some aspects, the rAAV is a single-stranded AAV (ssAAV) or a self-complementary AAV (scAAV).

In some aspects, the viral vector is an adeno-associated virus (AAV), such as an AAV1 (i.e., an AAV containing AAV1 inverted terminal repeats (ITRs) and AAV1 capsid proteins), AAV2 (i.e., an AAV containing AAV2 ITRs and AAV2 capsid proteins), AAV3 (i.e., an AAV containing AAV3 ITRs and AAV3 capsid proteins), AAV4 (i.e., an AAV containing AAV4 ITRs and AAV4 capsid proteins), AAV5 (i.e., an AAV containing AAV5 ITRs and AAV5 capsid proteins), AAV6 (i.e., an AAV containing AAV6 ITRs and AAV6 capsid proteins), AAV7 (i.e., an AAV containing AAV7 ITRs and AAV7 capsid proteins), AAV8 (i.e., an AAV containing AAV8 ITRs and AAV8 capsid proteins), AAV9 (i.e., an AAV containing AAV9 ITRs and AAV9 capsid proteins), AAVrh74 (i.e., an AAV containing AAVrh74 ITRs and AAVrh74 capsid proteins), AAVrh.8 (i.e., an AAV containing AAVrh.8 ITRs and AAVrh.8 capsid proteins), AAVrh.10 (i.e., an AAV containing AAVrh.10 ITRs and AAVrh.10 capsid proteins), AAV11 (i.e., an AAV containing AAV11 ITRs and AAV11 capsid proteins), AAV12 (i.e., an AAV containing AAV12 ITRs and AAV12 capsid proteins), AAV13 (i.e., an AAV containing AAV13 ITRs and AAV13 capsid proteins), AAV-anc80, AAV rh.74, AAV rh.8, AAVrh.10, or AAV-B1.

AAV is a replication-deficient parvovirus, the single-stranded DNA genome of which is about 4.7 kb in length including 145 nucleotide inverted terminal repeat (ITRs). There are multiple serotypes of AAV, for example, as set out herein above. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively (see also U.S. Pat. Nos. 7,282,199 and 7,790,449 relating to AAV-8); the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA. In some aspects, the rep and cap proteins are provided in trans. Another significant feature of AAV is that it is an extremely stable and hearty virus. It easily withstands the conditions used to inactivate adenovirus (56° to 65° C. for several hours), making cold preservation of AAV less critical. AAV may even be lyophilized. Finally, AAV-infected cells are not resistant to superinfection.

In some aspects, DNA plasmids of the disclosure comprise rAAV genomes of the disclosure. The DNA plasmids are transferred to cells permissible for infection with a helper virus of AAV (e.g., adenovirus, E1-deleted adenovirus or herpes virus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV-anc80, AAV rh.74, AAV rh.8, and AAVrh.10. In some aspects, AAV DNA in the rAAV genomes is from any AAV serotype for which a recombinant virus can be derived including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV-anc80, AAV rh.74, AAV rh.8, AAVrh.10, and AAV-B1. Other types of rAAV variants, for example rAAV with capsid mutations, are also included in the disclosure. See, for example, Marsic et al., Molecular Therapy 22(11): 1900-1909 (2014). As noted above, the nucleotide sequences of the genomes of various AAV serotypes are known in the art. Use of cognate components is specifically contemplated. Production of pseudotyped rAAV is disclosed in, for example, WO 01/83692 which is incorporated by reference herein in its entirety.

In some aspects, recombinant AAV genomes of the disclosure comprise one or more AAV ITRs flanking a polynucleotide sequence, for example, one or more an antisense sequences that bind to key exon definition elements in the pre-mRNA, inhibiting the recognition of a specific exon by the spliceosome, leading to exclusion of the target exon of DMD from the mature RNA. Thus, in some aspects, rAAV genomes of the disclosure comprise one or more AAV ITRs flanking a polynucleotide encoding, for example, one or more DMD antisense sequences. Commercial providers such as Ambion Inc. (Austin, TX), Darmacon Inc. (Lafayette, CO), InvivoGen (San Diego, CA), and Molecular Research Laboratories, LLC (Herndon, VA) generate custom inhibitory RNA molecules. In addition, commercial kits are available to produce custom siRNA molecules, such as SILENCER™ siRNA Construction Kit (Ambion Inc., Austin, TX) or psiRNA System (InvivoGen, San Diego, CA).

In some aspects, therefore, the U7 snRNA gene comprising the DMD inhibitory nucleic acid is cloned into an rAAV vector. Thus, aspects of the disclosure include an rAAV genome comprising a nucleic acid comprising a nucleotide sequence or a combination of at least two nucleotide sequences selected from the group consisting of:
(a) a nucleotide sequence comprising at least 80% identity to the sequence set forth in any one of SEQ ID NOs: 1-20 and 26-29;
(b) a nucleotide sequence complementary to the nucleotide sequence comprising at least 80% identity to the sequence set forth in any one of SEQ ID NOs: 1-20 and 26-29;
(c) a nucleotide sequence comprising the sequence set forth in any one of SEQ ID NOS: 1-20 and 26-29;
(d) a nucleotide sequence complementary to the nucleotide sequence comprising the sequence set forth in any one of SEQ ID NOs: 1-20 and 26-29; and
(e) a nucleotide sequence which binds to the sequence set forth in any one of SEQ ID NOs: 21-25.

In some aspects, the rAAV genome comprises a nucleic acid comprising a combination of at least two, at least three, at least four, at least five, at least six, at least seven, at least eight, at least nine, or at least ten or more nucleotide sequences selected from the group consisting of:
(a) a nucleotide sequence comprising at least 80% identity to the sequence set forth in any one of SEQ ID NOs: 1-20 and 26-29;
(b) a nucleotide sequence complementary to the nucleotide sequence comprising at least 80% identity to the sequence set forth in any one of SEQ ID NOs: 1-20 and 26-29;
(c) a nucleotide sequence comprising the sequence set forth in any one of SEQ ID NOS: 1-20 and 26-29;
(d) a nucleotide sequence complementary to the nucleotide sequence comprising the sequence set forth in any one of SEQ ID NOs: 1-20 and 26-29; or
(e) a nucleotide sequence which binds to the sequence set forth in any one of SEQ ID NOs: 21-25.

In some aspects, the viral vector is a pseudotyped AAV, containing ITRs from one AAV serotype and capsid proteins from a different AAV serotype. In some aspects, the pseudo-typed AAV is AAV2/9 (i.e., an AAV containing AAV2 ITRs and AAV9 capsid proteins). In some aspects, the pseudotyped AAV is AAV2/8 (i.e., an AAV containing AAV2 ITRs and AAV8 capsid proteins). In some aspects, the pseudotyped AAV is AAV2/1 (i.e., an AAV containing AAV2 ITRs and AAV1 capsid proteins).

In some aspects, the AAV contains a recombinant capsid protein, such as a capsid protein containing a chimera of one or more of capsid proteins from AAV1, AAV2, AAV3, AAV4, AAV5, AAV6, AAV7, AAV8, AAV9, AAV-anc80, AAVrh74, AAVrh.8, or AAVrh.10, AAV10, AAV11, AAV12, AAV13, or AAV-B1. Other types of rAAV variants, for example rAAV with capsid mutations, are also contemplated. See, for example, Marsic et al., Molecular Therapy, 22(11): 1900-1909 (2014). As set out herein above, the nucleotide sequences of the genomes of various AAV serotypes are known in the art.

In some aspects, DNA plasmids are provided which comprise rAAV genomes as described herein. The DNA plasmids are transferred to cells permissible for infection with a helper virus of AAV (e.g., adenovirus, E1-deleted adenovirus or herpesvirus) for assembly of the rAAV genome into infectious viral particles. Techniques to produce rAAV particles, in which an AAV genome to be packaged, rep and cap genes, and helper virus functions are provided to a cell are standard in the art. Production of rAAV requires that the following components are present within a single cell (denoted herein as a packaging cell): a rAAV genome, AAV rep and cap genes separate from (i.e., not in) the rAAV genome, and helper virus functions. The AAV rep and cap genes may be from any AAV serotype for which recombinant virus can be derived and may be from a different AAV serotype than the rAAV genome ITRs, including, but not limited to, AAV serotypes AAV-1, AAV-2, AAV-3, AAV-4, AAV-5, AAV-6, AAV-7, AAV-8, AAV-9, AAV-10, AAV-11, AAV-12, AAV-13, AAV-B1 and AAV rh74. Production of pseudotyped rAAV is disclosed in, for example, WO 01/83692 which is incorporated by reference herein in its entirety.

In some aspects, packaging cells are provided. Packaging cells are created in order to have a cell line that stably expresses all the necessary components for AAV particle production. Retroviral vectors are created by removal of the retroviral gag, pol, and env genes. These are replaced by the therapeutic gene. In order to produce vector particles, a packaging cell is essential. Packaging cell lines provide all the viral proteins required for capsid production and the virion maturation of the vector. Thus, packaging cell lines are made so that they contain the gag, pol and env genes. Following insertion of the desired gene into in the retroviral DNA vector, and maintenance of the proper packaging cell line, it is now a simple matter to prepare retroviral vectors

For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy & Carter, 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

Thus, further provided in some aspects are packaging cells that produce infectious rAAV. Packaging cells may be stably transformed cancer cells such as Hela cells, 293 cells and PerC.6 cells (a cognate 293 line). In another aspect, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

In some aspects, therefore, a method of generating a packaging cell to create a cell line that stably expresses all the necessary components for AAV particle production is provided. For example, a plasmid (or multiple plasmids) comprising a rAAV genome lacking AAV rep and cap genes, AAV rep and cap genes separate from the rAAV genome, and a selectable marker, such as a neomycin resistance gene, are integrated into the genome of a cell. AAV genomes have been introduced into bacterial plasmids by procedures such as GC tailing (Samulski et al., 1982, Proc. Natl. Acad. S6. USA, 79:2077-2081), addition of synthetic linkers containing restriction endonuclease cleavage sites (Laughlin et al., 1983, Gene, 23:65-73) or by direct, blunt-end ligation (Senapathy et al., 1984, J. Biol. Chem., 259:4661-4666). The packaging cell line is then infected with a helper virus such as adenovirus. The advantages of this method are that the cells are selectable and are suitable for large-scale production of rAAV. Other examples of suitable methods employ adenovirus or baculovirus rather than plasmids to introduce rAAV genomes and/or rep and cap genes into packaging cells.

General principles of rAAV production are reviewed in, for example, Carter, 1992, Current Opinions in Biotechnology, 1533-539; and Muzyczka, 1992, Curr. Topics in Microbiol. and Immunol. 158:97-129). Various approaches are described in Ratschin et al., Mol. Cell. Biol. 4:2072 (1984); Hermonat et al., Proc. Natl. Acad. Sci. USA, 81:6466 (1984); Tratschin et al., Mo1. Cell. Biol. 5:3251 (1985); Mclaughlin et al., J. Virol., 62:1963 (1988); and Lebkowski et al., 1988 Mol. Cell. Biol., 7:349 (1988). Samulski et al., J. Virol., 63:3822-3828 (1989); U.S. Pat. No. 5,173,414; WO 95/13365 and corresponding U.S. Pat. No. 5,658,776; WO 95/13392; WO 96/17947; PCT/US98/18600; WO 97/09441 (PCT/US96/14423); WO 97/08298 (PCT/US96/13872); WO 97/21825 (PCT/US96/20777); WO 97/06243 (PCT/FR96/01064); WO 99/11764; Perrin et al., Vaccine, 13:1244-1250 (1995); Paul et al., Human Gene Therapy, 4:609-615 (1993); Clark et al., Gene Therapy, 3:1124-1132 (1996); U.S. Pat. Nos. 5,786,211; 5,871,982; 6,258,595; and McCarty, Mol. Ther., 16(10): 1648-1656 (2008). The foregoing documents are hereby incorporated by reference in their entirety herein, with particular emphasis on those sections of the documents relating to rAAV production. The production and use of various types of rAAV are specifically contemplated and exemplified. Recombinant AAV (i.e., infectious encapsidated rAAV particles) are thus provided herein. In some aspects, genomes of the rAAV lack AAV rep and cap genes; that is, there is no AAV rep or cap DNA between the ITRs of the genomes of the rAAV. In some aspects, the AAV is a recombinant linear AAV (rAAV), a single-stranded AAV (ssAAV), or a recombinant self-complementary AAV (scAAV).

The disclosure thus provides packaging cells that produce infectious rAAV. In one aspect, packaging cells are stably transformed cancer cells, such as Hela cells, 293 cells and PerC.6 cells (a cognate 293 line). In another aspect, packaging cells are cells that are not transformed cancer cells, such as low passage 293 cells (human fetal kidney cells transformed with E1 of adenovirus), MRC-5 cells (human fetal fibroblasts), WI-38 cells (human fetal fibroblasts), Vero cells (monkey kidney cells) and FRhL-2 cells (rhesus fetal lung cells).

The rAAV, in some aspects, are purified by methods standard in the art, such as by column chromatography or cesium chloride gradients. Methods for purifying rAAV vectors from helper virus are known in the art and include methods disclosed in, for example, Clark et al., Hum. Gene Ther., 10(6): 1031-1039 (1999); Schenpp and Clark, Methods Mol. Med., 69 427-443 (2002); U.S. Pat. No. 6,566,118 and WO 98/09657.

In some aspects, the disclosure provides a composition or compositions comprising a nucleic acid or a vector, e.g., such as a viral vector, as described herein. Thus, compositions comprising delivery vehicles (such as rAAV) described herein are provided. In various aspects, such compositions also comprise a pharmaceutically acceptable carrier. In various aspects, such compositions also comprise other ingredients, such as a diluent, excipients, and/or adjuvant. Acceptable carriers, diluents, excipients, and adjuvants are nontoxic to recipients and are preferably inert at the dosages and concentrations employed, and include buffers such as phosphate, citrate, or other organic acids; antioxidants such as ascorbic acid; low molecular weight polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as Tween, pluronics or polyethylene glycol (PEG).

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

Titers of rAAV to be administered in methods of the disclosure will vary depending, for example, on the particular rAAV, the mode of administration, the treatment goal, the individual, and the cell type(s) being targeted, and may be determined by methods standard in the art. Titers of rAAV may range from about 1×10⁶, about 1×10⁷, about 1×10⁸, about 1×10⁹, about 1×10¹⁰, about 1×10¹¹, about 1×10¹², about 1×10¹³, about 1×10¹⁴, about 1×10¹⁵, about 1×10¹⁶, or more DNase resistant particles (DRP) [or viral genomes (vg)] per ml.

In some aspects, the dose of rAAV administered is about 1.0×10¹⁰ vg/kg to about 1.0×10¹⁶ vg/kg. In some aspects, 1.0×10¹⁰ vg/kg is also designated 1.0 E10 vg/kg, which is simply an alternative way of indicating the scientific notation. Likewise, 1011 is equivalent to E11, and the like. In some aspects, the dose of rAAV administered is about 1.0×10¹¹ vg/kg to about 1.0×10¹⁵ vg/kg. In some aspects the dose of rAAV is about 1.0×10¹⁰ vg/kg, about 2.0×10¹⁰ vg/kg, about 3.0×10¹⁰ vg/kg, about 4.0×10¹⁰ vg/kg, about 5.0×10¹⁰ vg/kg, about 6.0×10¹⁰ vg/kg, about 7.0×10¹⁰ vg/kg, about 8.0×10¹⁰ vg/kg, about 9.0×10¹⁰ about 1.0×10¹¹ vg/kg, about 2.0×10¹¹ vg/kg, about 3.0×10¹¹ vg/kg, about 4.0×10¹¹ vg/kg, about 5.0×10¹¹ vg/kg, about 6.0×10¹¹ vg/kg, about 7.0×10¹¹ vg/kg, about 8.0×10¹¹ vg/kg, about 9.0×10¹¹ vg/kg, about 1.0×10¹² vg/kg, about 2.0×10¹² vg/kg, about 3.0×10¹² vg/kg, about 4.0×10¹² vg/kg, about 5.0×10¹² vg/kg, about 6.0×10¹² vg/kg, about 7.0×10¹² vg/kg, about 8.0×10¹² vg/kg, about 9.0×10¹² vg/kg, about 1.0×10¹³ vg/kg, about 2.0×10¹³ vg/kg, about 3.0×10¹³ vg/kg, about 4.0×10¹³ vg/kg, about 5.0×10¹³ vg/kg, about 6.0×10¹³ vg/kg, about 7.0×10¹³ vg/kg, about 8.0×10¹³ vg/kg, about 9.0×10¹³ vg/kg, about 1.0×10¹⁴ vg/kg, about 2.0×10¹⁴ vg/kg, about 3.0×10¹⁴ vg/kg, about 4.0×10¹⁴ vg/kg, about 5.0×10¹⁴ vg/kg, about 6.0×10¹⁴ vg/kg, about 7.0×10¹⁴ vg/kg, about 8.0×10¹⁴ vg/kg, about 9.0×10¹⁴ vg/kg, about 1.0×10¹⁵ vg/kg, about 2.0×10¹⁵ vg/kg, about 3.0×10¹⁵ vg/kg, about 4.0×10¹⁵ vg/kg, about 5.0×10¹⁵ vg/kg, about 6.0×10¹⁵ vg/kg, about 7.0×10¹⁵ vg/kg, about 8.0×10¹⁵ vg/kg, about 9.0×10¹⁵ vg/kg, or about 1.0×10¹⁶ vg/kg.

In some aspects, the dose is about 1.0×10¹¹ vg/kg to about 1.0×10¹⁵ vg/kg. In some aspects, the dose is about 1.0×10¹¹ vg/kg to about 5.0×10¹⁴ vg/kg. In some aspects, the dose is about 1.0×10¹¹ vg/kg to about 1.0×10¹⁴ vg/kg. In some aspects, the dose is about 1.0×10¹¹ vg/kg to about 5.0×10¹³ vg/kg. In some aspects, the dose is about 1.0×10¹¹ vg/kg to about 1.0×10¹³ vg/kg. In some aspects, the dose is about 1.0×10¹¹ vg/kg to about 5.0×10¹² vg/kg. In some aspects, the dose is about 1.0×10¹¹ vg/kg to about 1.0×10¹² vg/kg. In some aspects, an initial dose is followed by a second greater dose. In some aspects, an initial dose is followed by a second same dose. In some aspects, an initial dose is followed by one or more lesser doses. In some aspects, an initial dose is followed by multiple doses which are the same or greater doses.

Methods of transducing a target cell with a delivery vehicle (such as rAAV), in vivo or in vitro, are contemplated. Transduction of cells with an rAAV of the disclosure results in sustained expression of antisense sequence that binds to key exon definition elements in the pre-mRNA of the DMD gene, inhibiting the recognition of a specific exon by the spliceosome, leading to exclusion of the target exon from the mature DMD RNA. The disclosure thus provides rAAV and methods of administering/delivering rAAV which express antisense sequence that binds to key exon definition elements in the pre-mRNA, inhibiting the recognition of a specific exon by the spliceosome, leading to exclusion of the target exon from the mature RNA to a subject. In some aspects, the subject is a mammal. In some aspects, the mammal is a human. These methods include transducing cells and tissues (including, but not limited to, tissues such as muscle) with one or more rAAV described herein. Transduction may be carried out with gene cassettes comprising cell-specific control elements. The term “transduction” is used to refer to, as an example, the administration/delivery of u7snRNA comprising antisense sequence to a target cell either in vivo or in vitro, via a replication-deficient rAAV described herein resulting in the expression of functional forms of the dystrophin protein by the target cell.

The in vivo methods comprise the step of administering an effective dose, or effective multiple doses, of a composition comprising a delivery vehicle (such as rAAV) to a subject (including a human subject) in need thereof. Thus, methods are provided of administering an effective dose (or doses, administered essentially simultaneously or doses given at intervals) of rAAV described herein to a subject in need thereof. If the dose or doses is administered prior to development of a disorder/disease, the administration is prophylactic. If the dose or doses is administered after the development of a disorder/disease, the administration is therapeutic. An effective dose is a dose that alleviates (eliminates or reduces) at least one symptom associated with the disorder/disease state being treated, that slows or prevents progression to a disorder/disease state, that slows or prevents progression of a disorder/disease state, that diminishes the extent of disease, that results in remission (partial or total) of disease, and/or that prolongs survival.

In some aspects, compositions and methods of the disclosure are used in treating, ameliorating, or preventing a disease, such as a muscular dystrophy (MD). In various aspects, such MD is Duchenne Muscular Dystrophy (DMD). DMD, an X-linked degenerative muscle disorder, is the most common severe childhood form of muscular dystrophy affecting around 1:5200 male births (Mendell et al., Ann Neurol 71, 304-313 (2012)). Symptoms of generalized muscle weakness first appear at ages 3-5 and progress into a loss of ambulation by age 13, with death typically occurring in the third decade of life due to cardiomyopathy or respiratory insufficiency (Passamano et al., Acta Myol 31, 121-125 (2012); Duchenne, The Pathology of Paralysis with Muscular Degeneration (Paralysie Myosclerotique), or Paralysis with Apparent Hypertrophy. Br Med J 2, 541-542 (1867)). DMD is caused by mutations that disrupt the open reading frame in the DMD gene, which encodes dystrophin (Juan-Mateu et al., PLOS One 10, e0135189 (2015)), a large (427 kDa) multifunctional protein that is localized at the subsarcolemmal region of myofibers, where it plays an important role in protecting the sarcolemma from mechanical damage caused by muscle contraction (Petrof et al., Proc Natl Acad Sci USA 90, 3710-3714 (1993)). In other various aspects, such MD is Becker Muscular Dystrophy (BMD). The presence of a partially functional dystrophin protein occurs with mutations that maintain an open reading frame (ORF), resulting in the milder allelic disorder BMD (Wein et al., Nature Medicine 20, 992-1000 (2014); Monaco, Trends Biochem Sci 14, 412-415 (1989)). BMD, like DMD, is a genetic disorder that gradually makes the body's muscles weaker and smaller. BMD affects the muscles of the hips, pelvis, thighs, and shoulders, as well as the heart, but is known to cause less severe problems than DMD. Because of the variety of in-frame mutations resulting in a variety of partially functional proteins, BMD has a broad phenotypic spectrum with, for example, loss of ambulation ranging from the late teenage years to late adulthood.

In families known to carry pathological DMD or BMD mutations, the methods of the disclosure, in various aspects, are methods of preventing disease and they are carried out before the onset of disease. In other various aspects, the methods of the disclosure are carried out after diagnosis and, therefore, are methods of treating or ameliorating disease.

Molecular, biochemical, histological, and functional outcome measures demonstrate the therapeutic efficacy of the methods. Outcome measures are described, for example, in Chapters 32, 35 and 43 of Dyck and Thomas, Peripheral Neuropathy, Elsevier Saunders, Philadelphia, PA, 4^{th} Edition, Volume 1 (2005) and in Burgess et al., Methods Mol. Biol., 602: 347-393 (2010). Outcome measures include, but are not limited to, one or more of the exclusion of the target exon from the mature RNA, reduction or elimination of mutant DMD mRNA or protein in affected tissues, and the expression of a functional form of dystrophin. The expression of functional dystrophin in the cell is detected by measuring the dystrophin protein level by methods known in the art including, but not limited to, Western blot, immunofluorescence, or immunohistochemistry in muscle biopsied before and after administration of the rAAV to determine the improvement.

In some aspects, the level of functional dystrophin gene expression or protein expression in a cell of the subject is increased after administration of the rAAV as compared to the level of functional dystrophin gene expression or protein expression before administration of the rAAV. In some aspects, expression of a functional form of dystrophin is increased by at least about 10%, at least about 20%, at least about 30%, at least about 40%, at least about 50%, at least about 60%, at least about 70%, at least about 80%, at least about 90%, at least about 95%, at least about 98%, at least about 99%, at least about 100% percent, or at least about greater than 100%. In various aspects, improved muscle strength, improved muscle function, and/or improved mobility and stamina show an improvement by at least about 2%, at least about 5%, at least about 10%, at least about 20%, at least about 30%, at least about 40%, at least about 50%, at least about 60%, at least about 70%, at least about 80%, at least about 90%, at least about 95%, at least about 98%, at least about 99%, at least about 100% percent, or at least about greater than 100%.

Other outcome measures include measuring the level of serum creatinine kinase (CK) in the subject before and after treatment. Increased CK levels are a hallmark of muscle damage. In Duchenne patients, CK levels are significantly increased above the normal range (10 to 100 times the normal level since birth). When elevated CK levels are found in a blood sample, it usually means muscle is being disintegrated by some abnormal process, such as a muscular dystrophy or inflammation. Thus, a positive therapeutic outcome for treatment with the methods of the disclosure is a reduction in the level of serum creatinine kinase after administration of the rAAV as compared to the level of serum creatinine kinase before administration of the rAAV.

Other outcome measure include measuring to determine if there is improved muscle strength, improved muscle function, improved mobility, improved stamina, or a combination of two or more thereof in the subject after treatment. Such outcome measures are important in determining muscular dystrophy progression in the subject and are measured by various tests known in the art. Some of these tests include, but are not limited to, the six minute walk test, time to rise test, ascend 4 steps test, ascend and descend 4 steps test, North Star Ambulatory Assessment (NSAA) test, 10 meter timed test, 100 meter timed test, hand held dynamometry (HHD) test, Timed Up and Go test, Gross Motor Subtest Scaled (Bayley-III) score, maximum isometric voluntary contraction test (MVICT), or a combination of two or more thereof.

Combination therapies are also contemplated by the disclosure. Combination as used herein includes both simultaneous treatment and sequential treatments. Combinations of methods described herein with standard medical treatments and supportive care are specifically contemplated, as are combinations with therapies, such as glucocorticoids. All types of glucocorticoids are included for use in the combination therapies disclosed herein. Such glucocorticoids include, but are not limited to, prednisone, prednisolone, dexamethasone, deflazacort, beclomethasone, betamethasone, budesonide, cortisone, hydrocortisone, methylprednisolone, and triamcinolone.

Administration of an effective dose of a nucleic acid, viral vector, or composition of the disclosure may be by routes standard in the art including, but not limited to, intramuscular, parenteral, intravascular, intravenous, oral, buccal, nasal, pulmonary, intracranial, intracerebroventricular, intrathecal, intraosseous, intraocular, rectal, or vaginal. In some aspects, an effective dose is delivered by a systemic route of administration, i.e., systemic administration. Systemic administration is a route of administration into the circulatory system so that the entire body is affected. Such systemic administration, in various aspects, takes place via enteral administration (absorption of the drug through the gastrointestinal tract) or parenteral administration (generally via injection, infusion, or implantation). In various aspects, an effective dose is delivered by a combination of routes. For example, in various aspects, an effective dose is delivered intravenously and/or intramuscularly, or intravenously and intracerebroventricularly, and the like. In some aspects, an effective dose is delivered in sequence or sequentially. In some aspects, an effective dose is delivered simultaneously. Route(s) of administration and serotype(s) of AAV components of the rAAV (in particular, the AAV ITRs and capsid protein) of the disclosure, in various aspects, are chosen and/or matched by those skilled in the art taking into account the condition or state of the disease or disorder being treated, the condition, state, or age of the subject, and the target cells/tissue(s) that are to express the nucleic acid or protein.

In particular, actual administration of delivery vehicle (such as rAAV) may be accomplished by using any physical method that will transport the delivery vehicle (such as rAAV) into a target cell of an animal. Administration includes, but is not limited to, injection into muscle, the bloodstream and/or directly into the nervous system or liver. Simply resuspending a rAAV in phosphate buffered saline has been demonstrated to be sufficient to provide a vehicle useful for muscle tissue expression, and there are no known restrictions on the carriers or other components that can be co-administered with the rAAV (although compositions that degrade DNA should be avoided in the normal manner with rAAV). Capsid proteins of a rAAV may be modified so that the rAAV is targeted to a particular target tissue of interest such as neurons. See, for example, WO 02/053703, the disclosure of which is incorporated by reference herein. Pharmaceutical compositions can be prepared as injectable formulations or as topical formulations to be delivered to the muscles by transdermal transport. Numerous formulations for both intramuscular injection and transdermal transport have been previously developed and can be used in the practice of the disclosure. The delivery vehicle (such as rAAV) can be used with any pharmaceutically acceptable carrier for ease of administration and handling.

A dispersion of delivery vehicle (such as rAAV) can also be prepared in glycerol, sorbitol, liquid polyethylene glycols and mixtures thereof and in oils. Under ordinary conditions of storage and use, these preparations contain a preservative to prevent the growth of microorganisms. In this connection, the sterile aqueous media employed are all readily obtainable by standard techniques known to those skilled in the art.

The pharmaceutical forms suitable for injectable use include sterile aqueous solutions or dispersions and sterile powders for the extemporaneous preparation of sterile injectable solutions or dispersions. In all cases the form must be sterile and must be fluid to the extent that easy syringeability exists. It must be stable under the conditions of manufacture and storage and must be preserved against the contaminating actions of microorganisms such as bacteria and fungi. The carrier can be a solvent or dispersion medium containing, for example, water, ethanol, polyol (for example, glycerol, propylene glycol, liquid polyethylene glycol, sorbitol and the like), suitable mixtures thereof, and vegetable oils. The proper fluidity can be maintained, for example, by the use of a coating such as lecithin, by the maintenance of the required particle size in the case of a dispersion and by the use of surfactants. The prevention of the action of microorganisms can be brought about by various antibacterial and antifungal agents, for example, parabens, chlorobutanol, phenol, sorbic acid, thimerosal and the like. In many cases it will be preferable to include isotonic agents, for example, sugars or sodium chloride. Prolonged absorption of the injectable compositions can be brought about by use of agents delaying absorption, for example, aluminum monostearate and gelatin.

Sterile injectable solutions are prepared by incorporating rAAV in the required amount in the appropriate solvent with various other ingredients enumerated above, as required, followed by filter sterilization. Generally, dispersions are prepared by incorporating the sterilized active ingredient into a sterile vehicle which contains the basic dispersion medium and the required other ingredients from those enumerated above. In the case of sterile powders for the preparation of sterile injectable solutions, the preferred methods of preparation are vacuum drying and the freeze drying technique that yield a powder of the active ingredient plus any additional desired ingredient from the previously sterile-filtered solution thereof.

The disclosure also provides a kit comprising a nucleic acid, vector, or composition of the disclosure or produced according to a process of the disclosure. In the context of the disclosure, the term “kit” means two or more components, one of which corresponds to a nucleic acid, vector, or composition of the disclosure, and the other which corresponds to a container, recipient, instructions, or otherwise. A kit, therefore, in various aspects, is a set of products that are sufficient to achieve a certain goal, which can be marketed as a single unit.

The kit may comprise one or more recipients (such as vials, ampoules, containers, syringes, bottles, bags) of any appropriate shape, size and material containing the nucleic acid, vector, or composition of the disclosure in an appropriate dosage for administration (see above). The kit may additionally contain directions or instructions for use (e.g. in the form of a leaflet or instruction manual), means for administering the nucleic acid, vector, or composition, such as a syringe, pump, infuser or the like, means for reconstituting the nucleic acid, vector, or composition and/or means for diluting the nucleic acid, vector, or composition.

In one embodiment, such a kit includes the nucleic acids or vectors in a diluent packaged in a container such as a sealed bottle or vessel, with a label affixed to the container or included in the package that describes use of the nucleic acids or vectors. In one embodiment, the diluent is in a container such that the amount of headspace in the container (e.g., the amount of air between the liquid formulation and the top of the container) is very small. Preferably, the amount of headspace is negligible (i.e., almost none).

In some aspects, the formulation comprises a stabilizer. The term “stabilizer” refers to a substance or excipient which protects the formulation from adverse conditions, such as those which occur during heating or freezing, and/or prolongs the stability or shelf-life of the formulation in a stable state. Examples of stabilizers include, but are not limited to, sugars, such as sucrose, lactose and mannose; sugar alcohols, such as mannitol; amino acids, such as glycine or glutamic acid; and proteins, such as human serum albumin or gelatin.

In some aspects, the formulation comprises an antimicrobial preservative. The term “antimicrobial preservative” refers to any substance which is added to the composition that inhibits the growth of microorganisms that may be introduced upon repeated puncture of the vial or container being used. Examples of antimicrobial preservatives include, but are not limited to, substances such as thimerosal, 2-phenoxyethanol, benzethonium chloride, and phenol.

In some aspects, the kit comprises a label and/or instructions that describes use of the reagents provided in the kit. The kits also optionally comprise catheters, syringes or other delivering devices for the delivery of one or more of the compositions used in the methods described herein.

The disclosure also provides kits for a single dose of administration unit or for multiple doses. In some aspects, the disclosure provides kits containing single-chambered and multi-chambered pre-filled syringes.

This entire document is intended to be related as a unified disclosure, and it should be understood that all combinations of features described herein are contemplated, even if the combination of features are not found together in the same sentence, or paragraph, or section of this document. The disclosure also includes, for instance, all embodiments of the disclosure narrower in scope in any way than the variations specifically mentioned above. With respect to aspects of the disclosure described as a genus, all individual species are considered separate aspects of the disclosure. With respect to aspects of the disclosure described or claimed with “a” or “an,” it should be understood that these terms mean “one or more” unless context unambiguously requires a more restricted meaning. If aspects of the disclosure are described as “comprising” a feature, embodiments also are contemplated “consisting of” or “consisting essentially of” the feature.

Unless otherwise indicated, the term “at least” preceding a series of elements is to be understood to refer to every element in the series. Those skilled in the art will recognize, or be able to ascertain using no more than routine experimentation, many equivalents to the specific aspects of the disclosure described herein. Such equivalents are intended to be encompassed by the disclosure.

The term “and/or” wherever used herein includes the meaning of “and”, “or” and “all or any other combination of the elements connected by said term.”

The term “about” or “approximately” as used herein means within 20%, preferably within 10%, and more preferably within 5% of a given value or range. It includes, however, also the concrete number, e.g., about 10 includes 10. Recitation of ranges of values herein are merely intended to serve as a shorthand method for referring individually to each separate value falling within the range and each endpoint, unless otherwise indicated herein, and each separate value and endpoint is incorporated into the specification as if it were individually recited herein.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or “including” or sometimes when used herein with the term “having.”

When used herein, “consisting of” excludes any element, step, or ingredient not specified in the claim element. When used herein, “consisting essentially of” does not exclude materials or steps that do not materially affect the basic and novel characteristics of the claim.

In each instance herein any of the terms “comprising”, “consisting essentially of” and “consisting of” may be replaced with either of the other two terms.

It should be understood that this disclosure is not limited to the particular methodology, protocols, material, reagents, and substances, etc., described herein and as such can vary. The terminology used herein is for the purpose of describing particular aspects only, and is not intended to limit the scope of the subject matter of the disclosure, which is defined solely by the claims.

All methods described herein are performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All publications and patents cited throughout the text of this specification (including all patents, patent applications, scientific publications, manufacturer's specifications, instructions, etc.), whether supra or infra, are hereby incorporated by reference in their entirety. To the extent the material incorporated by reference contradicts or is inconsistent with this specification, the specification will supersede any such material.

A better understanding of the disclosure and of its advantages will be obtained from the following examples, offered for illustrative purposes only. The examples are not intended to limit the scope of the disclosure.

### EXAMPLES

Aspects and aspects of the disclosure are illustrated by the following examples.

### Example 1

### Materials and Methods

### Constructs

The DNA encoding the U7 snRNA gene constructs comprising the promoter, antisense, smOPT, loop, and 3′UTR sequences, as set out in Tables 2 and 3, were cloned into AAV vectors. All plasmid constructs were sequence verified.

### U7snRNA AAV Vector Design and Production

Vectors were produced in the Viral Vector Core at The Research Institute at Nationwide Children's Hospital. Serotypes 1 and 9 recombinant adeno-associated virus (rAAV1; rAAV9) vectors were produced by a modified cross-packaging approach using an adenovirus-free, triple plasmid DNA transfection (CaPO4 precipitation) method in human embryonic kidney 293 cells (Rabinowitz et al., J. Virol. 2002; 76: 791-801).

Immortalized and Conditionally Inducible fibroMyoD Cell Lines

Expression of the MyoD gene in mammalian fibroblasts results in transdifferentiation of cells into the myogenic lineage. Such cells can be further differentiated into myotubes, and they express muscle genes, including the DMD gene. Immortalized cell lines that conditionally express MyoD under the control of a tetracycline-inducible promoter were generated. This is achieved by stable transfection of the primary fibroblast lines of a lentivirus the tet-inducible MyoD and containing the human telomerase gene (TER). The resultant stable line allows MyoD expression to be initiated by treatment with doxycycline. Such cell lines were generated from patients with DMD who carry a mutation within exons 6, 7, and 8.

WT immortalized human fibroblasts that were able to transdifferentiate into muscle lineage cells under the control of doxycycline were produced by transduction with both telomerase-expressing and tet-inducible-MyoD expressing vectors [Chaouch, S., et al. Human gene therapy 20, 784-790 (2009)]. The converted human fibromyoblasts (FM) were then transduced with the scAAV1 vectors carrying different U7 constructs incorporating antisense sequences for exons 6, 7, and 8.

Hdmd7 (delCH2) Mouse Model

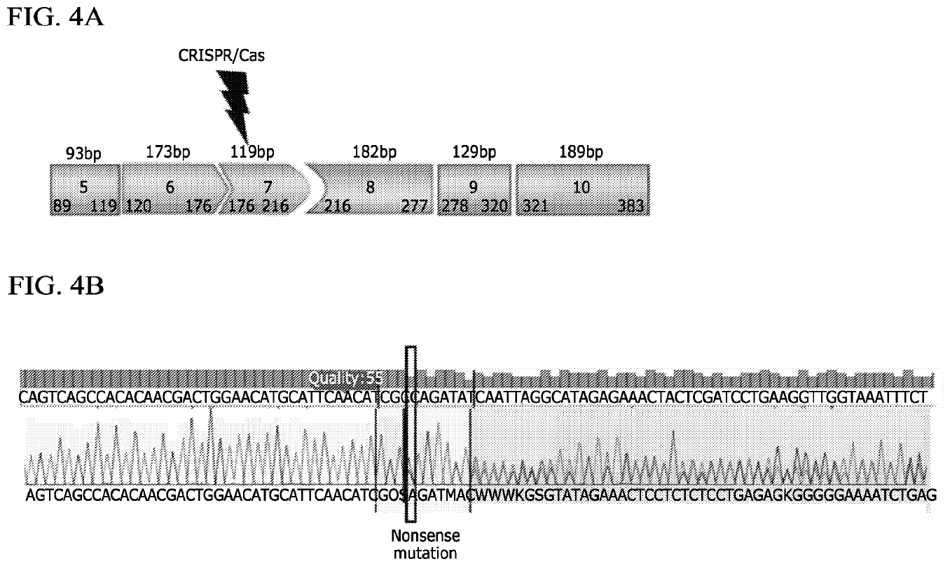

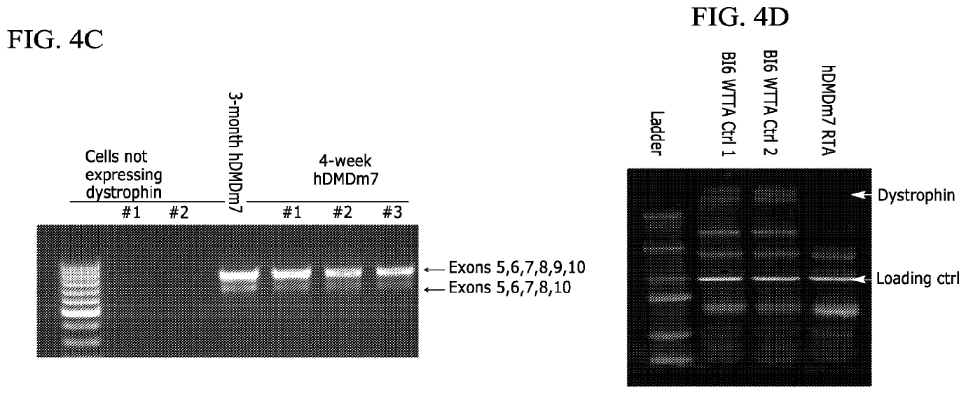

Mice carrying a nonsense mutation in hDMD exon 7 within the hdmd locus were developed using a CRISPR/Cas genome editing tool. A schematic showing the mutation is shown in FIG. 4A. In it, the numbers indicate the length of each exon and intron (the numbering in each exon (set out in red) correspond to the amino acid). PCR Amplicons from exons 5-10 and 5-9 differ by 129 bp (i.e., the length of exon 9).

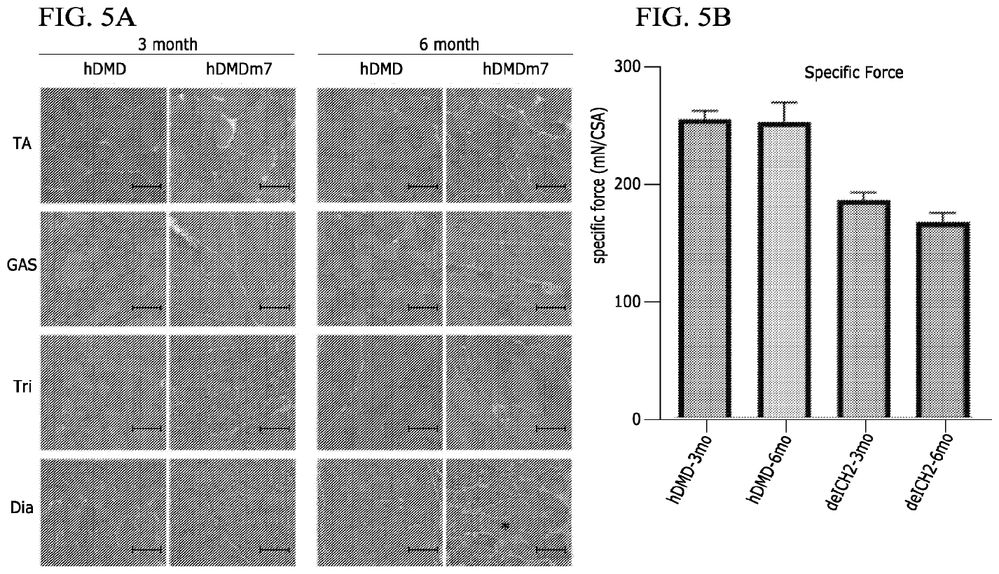

This hdmd7 (delCH2) mouse model presents an absence of dystrophin at the protein level and demonstrates centronucleation and a muscle force decrease (FIG. 5A-C). Multi-exon skipping of exons 6, 7, and 8 was designed to restore the reading frame of this hDMDm7 mouse. As proven in the Golden Retriever Dog Model (GRDM) (Leguiner et al., 2014 November; 22(11):1923-35; Vulin et al., Mol Ther. 2012 November; 20(11):2120-33), skipping of exons 6-8 led to expression of a truncated but highly functional dystrophin. The dogs in those studies were able to express a transcript lacking exons 6-8 and were able to walk or run (Leguiner et al., 2014 (supra); Vulin et al., 2012 (supra).

Male C57BL/6 ES cells were transfected with CRISPR/Cas9 tool and with a template to introduce the nonsense mutation in exon 7 (see FIG. 4A-B). Insertion was checked by PCR. One good clone was found, amplified and injected in dozens of albino BL/6 blastocysts. Injected blastocysts were implanted into recipient mice. The dystrophin gene from chimeric males was checked by PCR and then by RT-PCR. The colony was expanded and maintained as heterozygous. FIG. 4C shows the absence of dystrophin expression in muscles from both the 4-week-old and 3-month-old hemizygous hDMDm7 mouse.

### Experimental Animal Use and Animal Studies

All experimental procedures involving animals in this study were reviewed and approved by the research institute at Nationwide Children's Hospital's Institutional Animal Care and Use Committee.

### Intramuscular and Tail Vein Injection

In some aspects, intramuscular injection was used. For the intramuscular injection studies, mice were injected into the tibialis anterior (TA) with a dose of vector DNA at 4.11E11 vector genomes (vg) and sacrificed one month later. At sacrifice, TA, gastrocnemius, triceps, diaphragm and heart were removed and frozen in liquid nitrogen-cooled isopentane.

In some aspects, injection is performed using a tail vein apparatus. The tail is warmed via light bulb to enlarge the veins. Once visible, the AAV vector or control (saline or phosphate-buffered saline (PBS)) is injected in 300 μl total of saline or PBS, or saline or PBS alone, using a 33G gas-tight Hamilton syringe. Following injection, a sterile cotton pad is placed on the injection site and held with pressure until bleeding ceases.

### Muscle Preparation

Mice dissection was using standard techniques. Muscles were collected and were either snap frozen or mounted for cryosections, which were cut at 10 μM for immunofluorescence and H&E staining. The tibialis anterior (ta), gastrocnemius (gastroc), quadriceps (quad) and triceps from the right side of all treatment groups were analyzed. Tissues/organs for histopathology studies were collected and fixed in 10% neutral buffered formalin (10% NBF).

### Exon Skipping Analysis

Total RNA was isolated from tissue samples and analyzed by reverse transcription (RT) followed by PCR. Total RNA was isolated from frozen muscle sections (15×40 μM sections) using Trizol according to the manufacturers protocol (Life Technologies, 15596018). For each sample, 1 μg of total RNA was used to generate cDNA by RT-PCR using random hexamer primers according to the manufacturer's protocol (Thermo Scientific, Ferk 1672) and then used for a single PCR of 35 cycles using 1 μg of RNA and a mixture of random hexamer and oligo(dT) for each RT reaction. mRNA was then amplified via primers specific to the regions analyzed. PCR amplification was performed using 2× Master Mix (Thermo Scientific, K0172) and 150 ng of RT product as a template. Following electrophoresis, PCR products were separated by electrophoresis on a 2% agarose gel and imaged using a Gel Logic 200 Imaging System (Kodak).

### Sanger Sequencing

For confirmation of the presence of point mutations and exon skipping products, amplified PCR were produced and ran through a 2% agarose gel. Bands were extracted using a Zymogen gel DNA recovery kit (D4001/D4002, Zymogen) according to standard protocol. DNA were then sent for Sanger sequencing to The Ohio State University Comprehensive Cancer Center Shared Resources Genomics Shared Resources where sequencing was carried out.

### Immunoblot Analysis

Protein extractions were conducted starting with 25 sections (40 μM) and 100 μl of lysis buffer containing a base buffer, a phosphatase inhibitor (PhosStop, Roche, 4906845001) and a protease inhibitor (Halt Protease Inhibitor Cocktail, Fisher, 78430). Steel beads were added to the tissue, which was homogenized using the Tissuelyser II (Qiagen) for 2 min at a rate of 30/sec. Lysates were then incubated on ice and spun down; the supernatant was removed and stored at −80° C. until immunoblotting, and the cell debris was discarded. For immunoblotting, 50-150 μg muscle tissue protein was loaded with a loading dye (1× laemmli) on a 3-8% Tris Acetate gel (Life Technologies, EA0378BOX). The gels were run for 30 min at 80 V, and then for 4 hours at 120 V. Protein was transferred to a nitrocellulose membrane (Fisher, 09-301-108) overnight at 4° C. in transfer buffer (Invitrogen, NP00061) at 50 V. The membranes were then exposed using a rat monoclonal anti-dystrophin primary antibody (1:200, Abcam, ab15277) and mouse monoclonal β-actinin primary antibody (1:5000, Fisher, MA122863) followed by IRDye α-rabbit 680 and α-mouse 800 (Licor, 926-68071 and 926-32210) secondary antibody. The membrane was scanned on the Odyssey CLx and imaged using Image Studio 14.

Force Generation and Protection from Eccentric Contractions

Physiologic studies were conducted on TA muscles from 3 month-old and 6-month old mice. The force study procedure was conducted using a modified version of Hakim's procedure (Hakim et al., J Appl. Physiol. (1985) 2011; 110:1656-1663; Wein et al., Nat. Med. 2014; 20:992-1000). Initial anesthetization of mice was conducted by giving an intraperitoneal injection of a cocktail containing five times their weight (i.e., the weight of the mouse) of 25 mg/mL ketamine and twice their weight of 2.5 mg/ml xylazine. The skin fascia and connective tissue were removed from around the tibialis anterior (TA). Throughout the procedure, the TA muscle was constantly moistened with 0.9% saline. A knot was tied to the distal TA tendon with a 4-0 suture and the tendon was cut. The excess suture thread was then knotted again, leaving a loop to attach the tendon to the force transducer. The mouse was then positioned on the platform which was kept at 37° for the duration of the experiment. The leg limb was secured to the platform by putting a pin through the knee cap and taping down the foot. The loop of suture attached to the TA tendon was then attached to a 205B dual-mode servomotor transducer (Aurora Scientific, Aurora, ON, Canada). Finally, two electrical probes were placed in the biceps femoral muscle near the sciatic nerve for stimulation.

The resting force was set to between 3-4 g for a 10 minute equilibrium period. The TA muscle was then stimulated at the optimal length (Lo, mm) and active tetanic muscle force was recorded to give the absolute force measurement using the Lab View-based DMC program (Aurora Scientific). The muscle was then run through a 10-step passive stretch protocol during which stimulation was applied to determine the force drop following repeated eccentric contractions. At each step, the TA muscle was passively strained 10% of the Lo. Once the protocol was complete, the mice were given a lethal dose of ketamine/xylazine and the TA muscle was removed and weighed. The cross-sectional area measured as mass (g)/(muscle density×ratio of fiber length×Lo) where muscle density is 1.06 mg/mm3 and the ratio of fiber length in the TA is 0.6 (Burkholder et al., J Morphol 1994; 221:177-190). The cross-sectional area using the muscle weight and Lo were applied to the absolute force measurement to give the specific force measurement. Statistical significance was assessed using a Kruskal-Wallace test assuming nonparametric data in the GraphPad Prism (version 6.03 for Windows, GraphPad Software, San Diego California USA). For all data, the mean and standard deviation were determined for each measurement and subsequently measurements that were more than +1 standard deviation were removed as outliers. The tenth recording for eccentric contractions was used to determine outliers.

### Statistical Analyses

Statistical analyses were performed using GraphPad Prism (GraphPad). Data normality was tested using D'agostino-Pearson when sample size was sufficient or using the Shapiro-Wilk test. When data had Gaussian distribution, a one-way ANOVA test was used. If not, a Kruskal-Wallis assay was performed. This methodology was applied for all experiments except for the eccentric contraction-induced damage (ECC) where a two-way ANOVA analysis, followed by Bonferroni post-hoc test, were performed.

### Example 2

### Antisense Sequences and Vectors

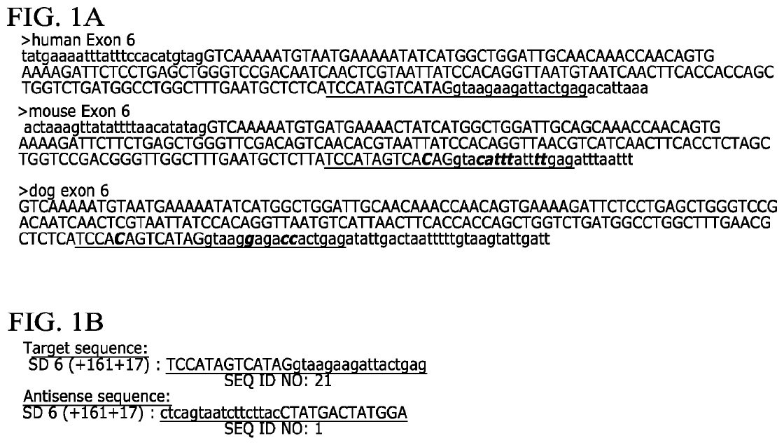

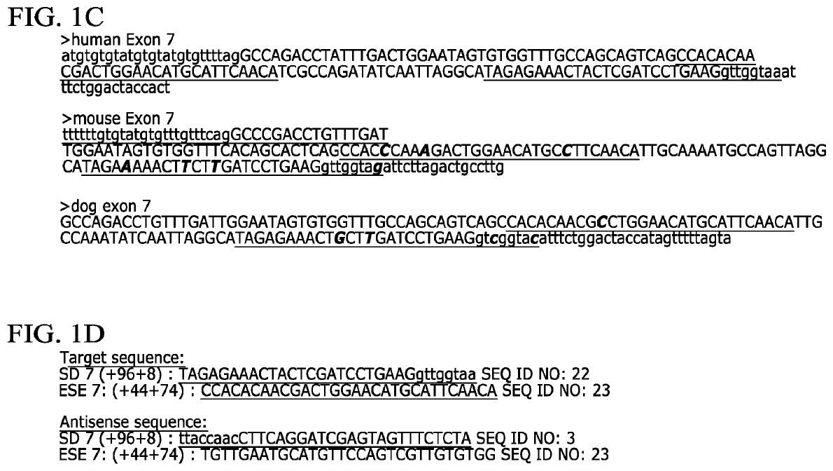

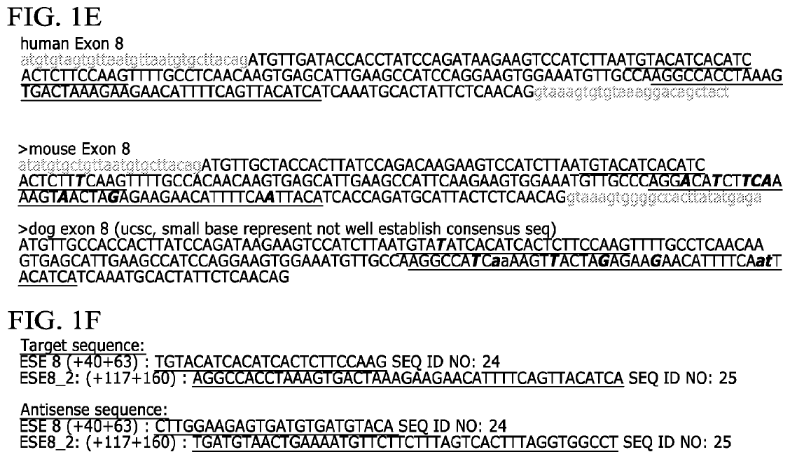

Products and methods for carrying out virally-mediated skipping of exons 6, 7 and 8 were developed. The products and methods were modified compared to the U7snRNA systems described in Goyenvalle et al., Science, 306(5702): 1796-1799 (2004) or Goyenvalle et al., Mol. Ther., 20(6): 179601799 (2004). U7snRNA was modified to include several target antisense sequences to interfere with splicing at exons 6, 7 and 8. Specifically, antisense sequences targeting exons 6, 7 and 8 were designed as shown in FIG. 1A-F and as set out in Table 4 below.

TABLE 4
Antisense Sequences.

| No. | Name | Sequence | SEQ ID NO: |
|---|---|---|---|
| 1 | SD6 antisense for U7 | ctcagtaatcttcttacCTATGACTATGGA | 1 |
| | Forward | | |
| 2 | SD6 antisense for U7 | TCCATAGTCATAGgtaagaagattactgag | 2 |
| | Reverse | | |
| 3 | SD7 antisense for U7 | ttaccaacCTTCAGGATCGAGTAGTTT | 3 |
| | Forward | CTCTA | |
| 4 | SD7 antisense for U7 | TAGAGAAACTACTCGATCCTGAAGgt | 4 |
| | Reverse | tggtaa | |
| 5 | ESE7 antisense for U7 | TGTTGAATGCATGTTCCAGTCGTTG | 5 |
| | Forward | TGTGG | |
| 6 | ESE7 antisense for U7 | CCACACAACGACTGGAACATGCATT | 6 |
| | Reverse | CAACA | |
| 7 | ESE8 antisense for U7 | CTTGGAAGAGTGATGTGATGTACA | 7 |
| | Forward | | |
| 8 | ESE8 antisense for U7 | TGTACATCACATCACTCTTCCAAG | 8 |
| | Reverse | | |
| 9 | ESE8_2 antisense for U7 | TGATGTAACTGAAAATGTTCTTCTTT | 9 |
| | Forward | AGTCACTTTAGGTGGCCT | |
| 10 | ESE8_2 antisense for U7 | AGGCCACCTAAAGTGACTAAAGAA | 10 |
| | Reverse | GAACATTTTCAGTTACATCA | |

U7 snRNA constructs comprising the antisense sequences targeting exons 6, 7 and 8 were generated. Each U7 snRNA construct included one of the target sequences. Self-complementary (sc) AAV vectors with genomes including one or more of the U7 snRNA constructs were then produced.

Recombinant scAAV vectors were produced by a modified cross-packaging approach using a plasmid comprising a desired vector genome by an adenovirus-free, triple plasmid DNA transfection (CaPO4 precipitation) method in HEK293 cells [Rabinowitz et al., J. Virol., 76:791-801 (2002)]. Vector was produced by co-transfecting with an AAV helper plasmid and an adenovirus helper plasmid in similar fashion as that previously described [Wang et al., Gene. Ther., 10:1528-1534 (2003)]. The adenovirus helper plasmid (pAdhelper) expresses the adenovirus type 5 E2A, E4ORF6, and VA I/II RNA genes which are required for high-titer rAAV production.

Vectors were purified from clarified 293 cell lysates by sequential iodixanol gradient purification and anion-exchange column chromatography using a linear NaCl salt gradient as previously described [Clark et al., Hum. Gene Ther, 10:1031-1039 (1999)]. Vector genome (vg) titers were measured using QPCR-based detection with a specific primer/probe set utilizing the Prism 7500 Taqman detector system (PE Applied Biosystems) as previously described (Clark et al., (supra)). Vector stock titers ranged between 1-10 ×10¹² vg/mL.

### Example 3

Effectiveness of U7 snRNA-Mediated Skipping on WT Cell Lines and a hDMD Mouse Model

Initial exon-skipping analysis was carried out by RT-PCR using self-complementary recombinant AAV (scrAAV) vectors to transduce WT immortalized human fibromyoblasts. WT immortalized human fibroblasts that were able to transdifferentiate into muscle lineage cells under the control of doxycycline were produced by transduction with both telomerase-expressing and tet-inducible-MyoD expressing vectors [Chaouch, S., et al. Human gene therapy 20, 784-790 (2009)]. The converted human fibromyoblasts (FM) were then transduced with the scrAAV vectors carrying different U7 constructs incorporating antisense sequences for exons 6, 7, and 8.

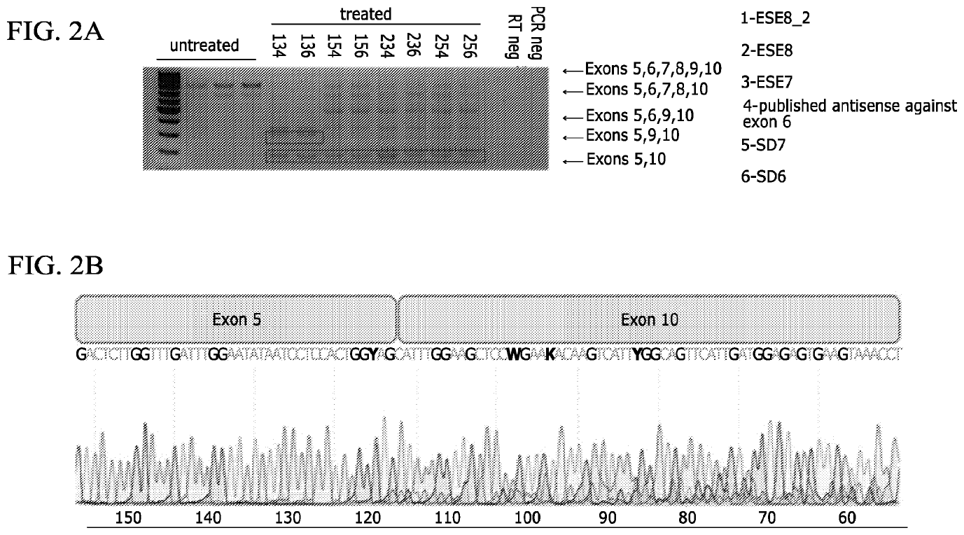

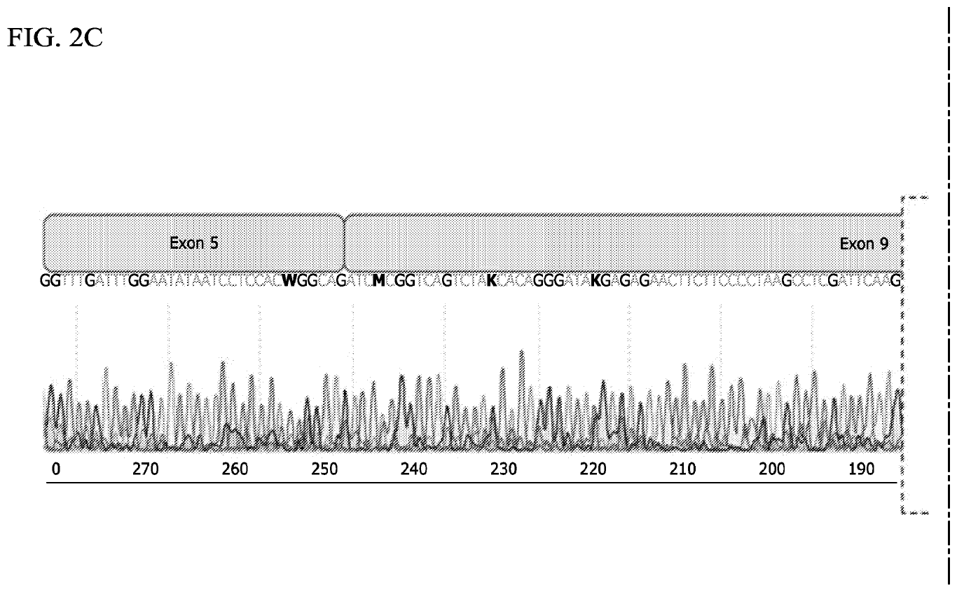

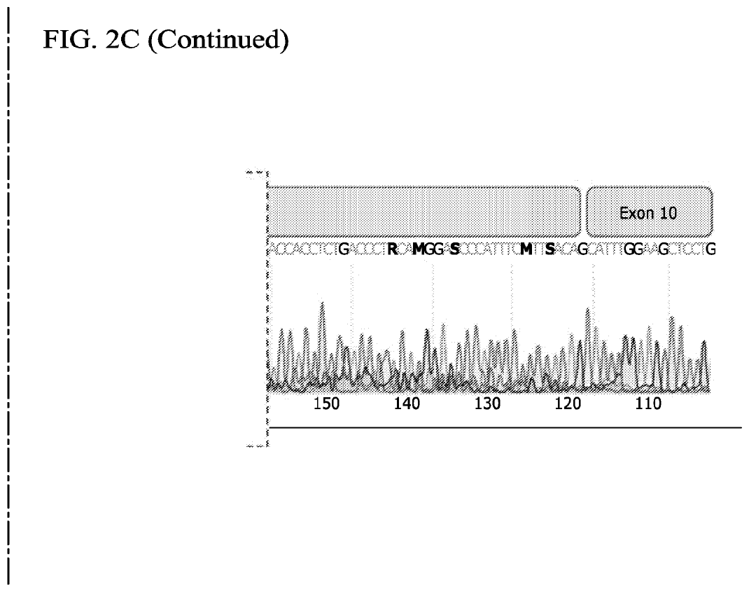

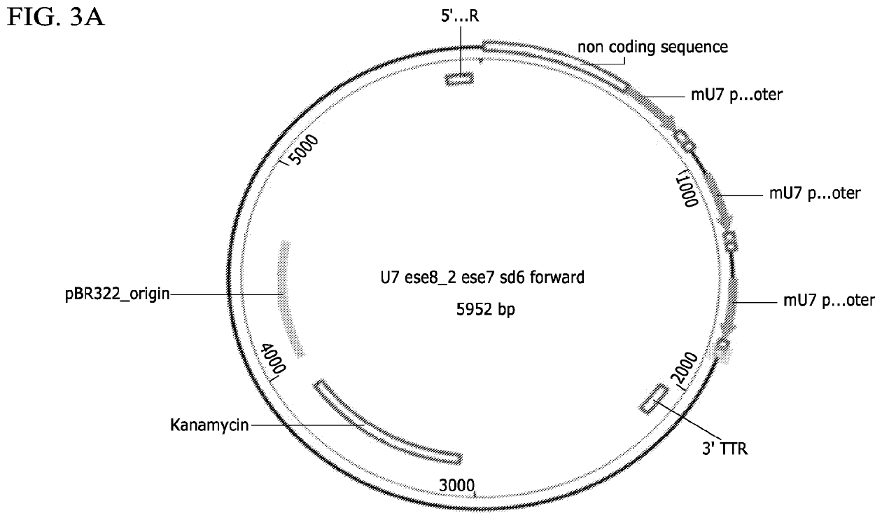

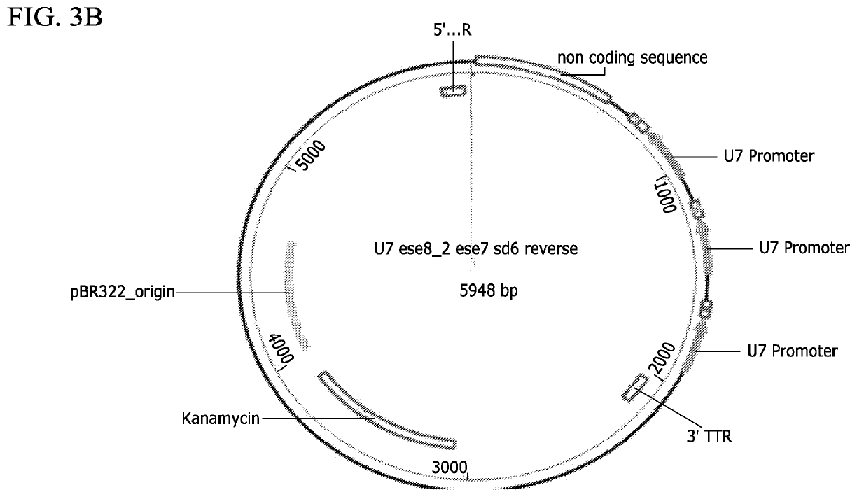

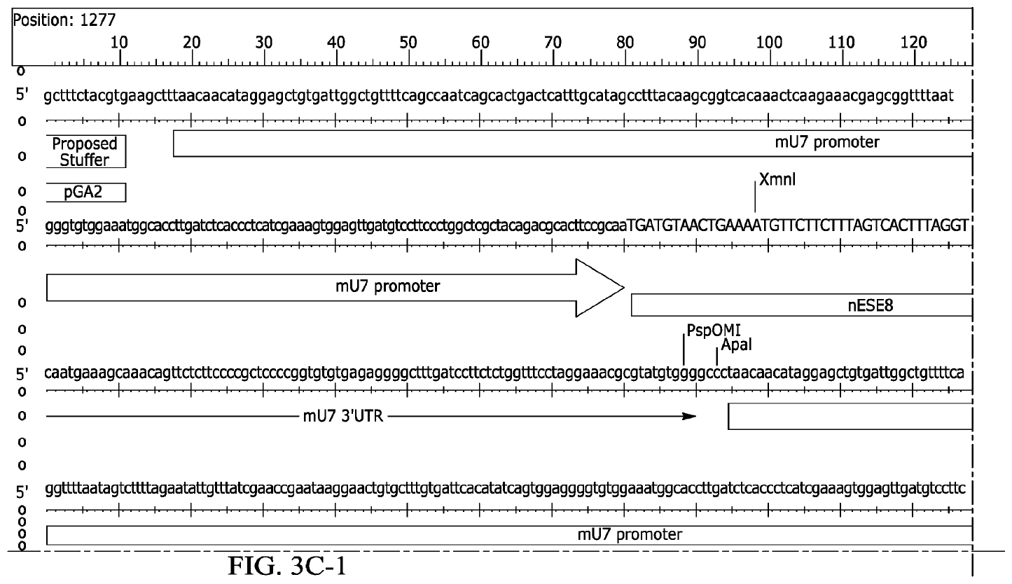

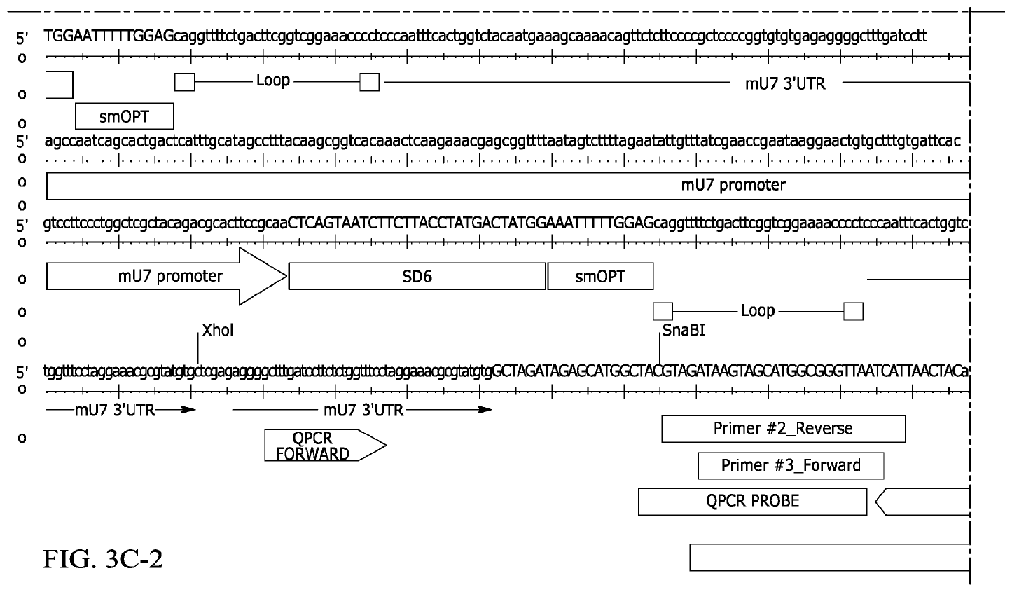

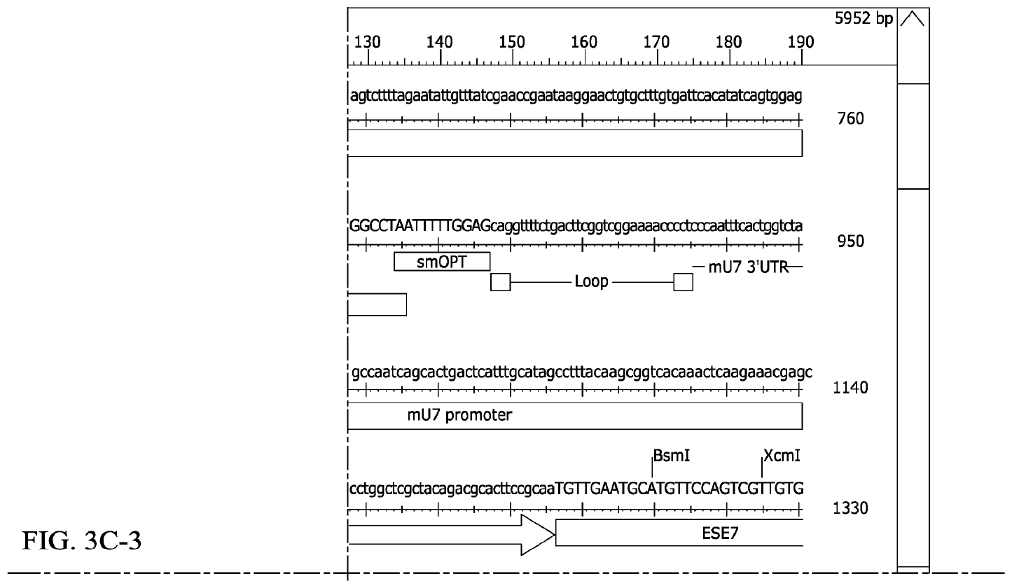

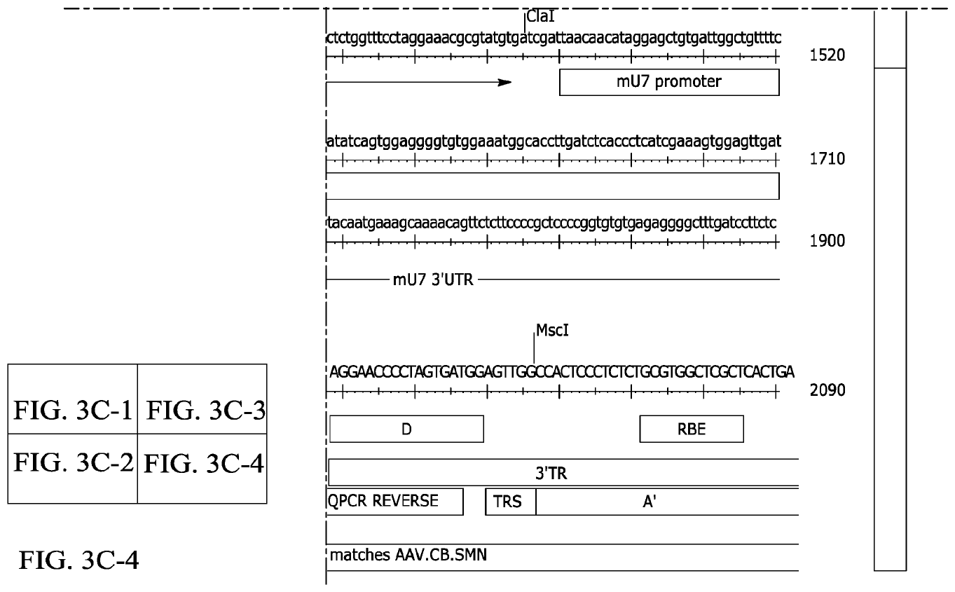

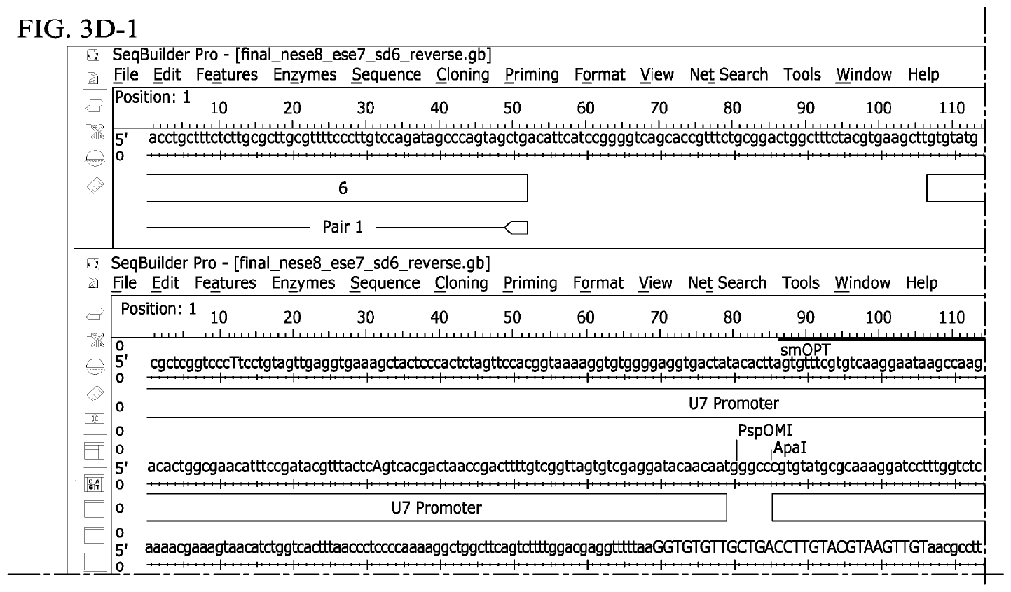

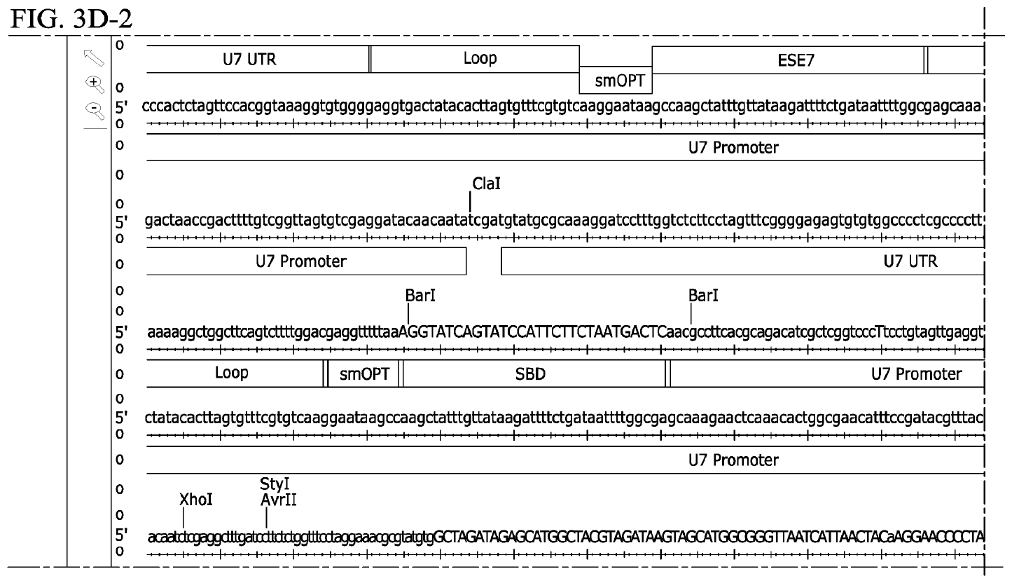

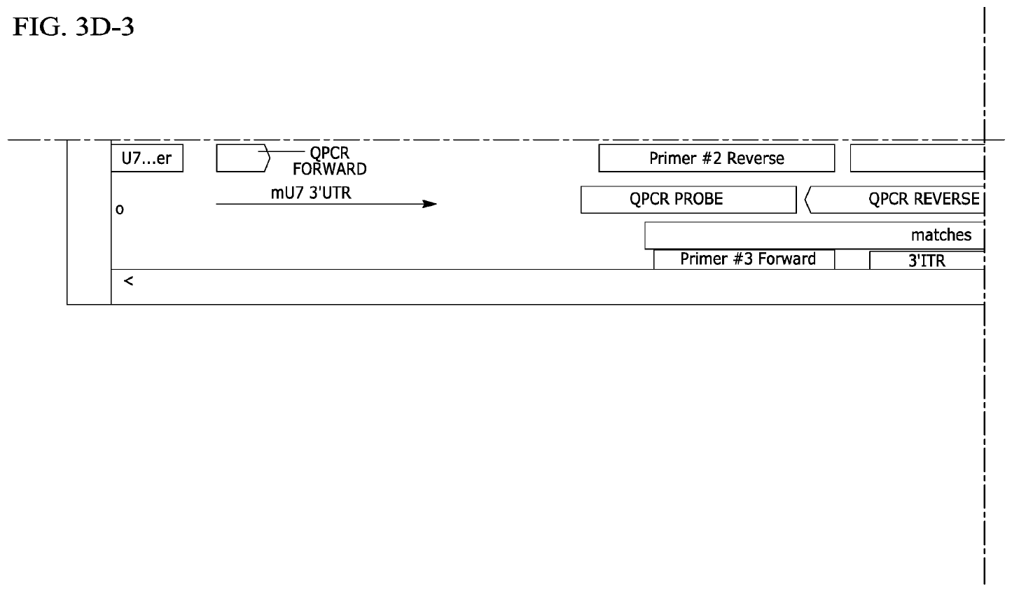

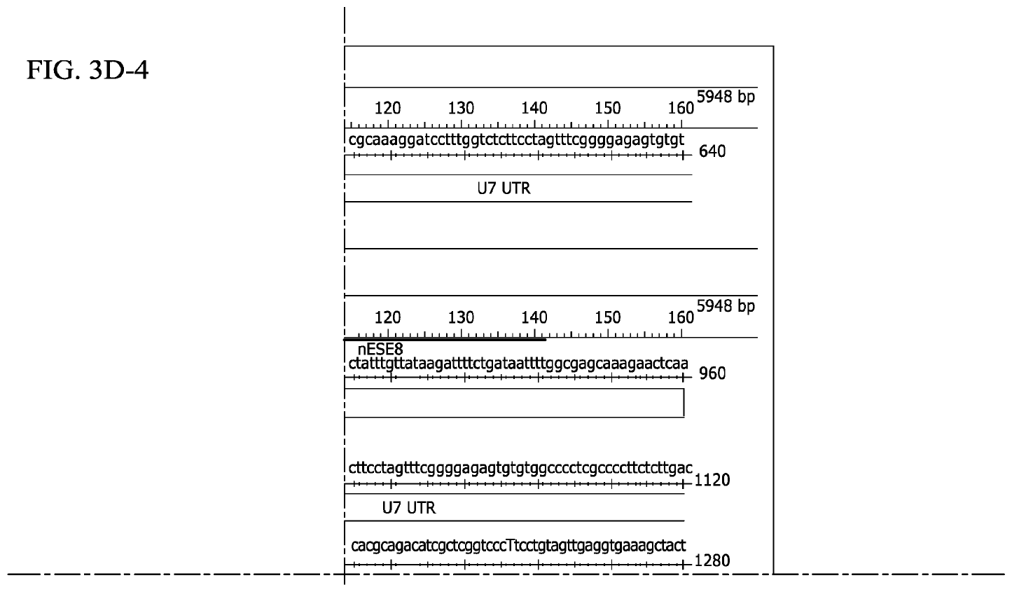

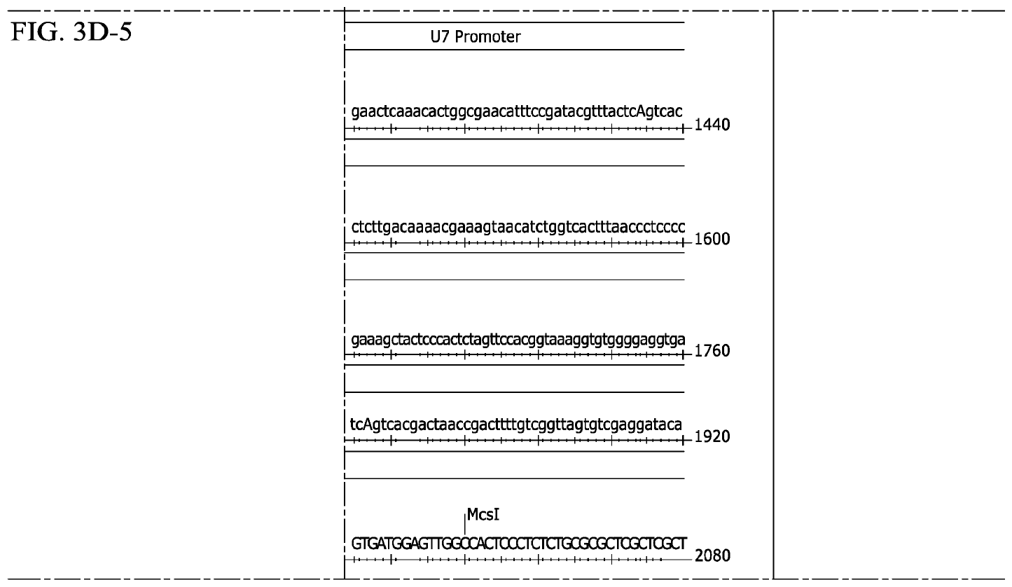

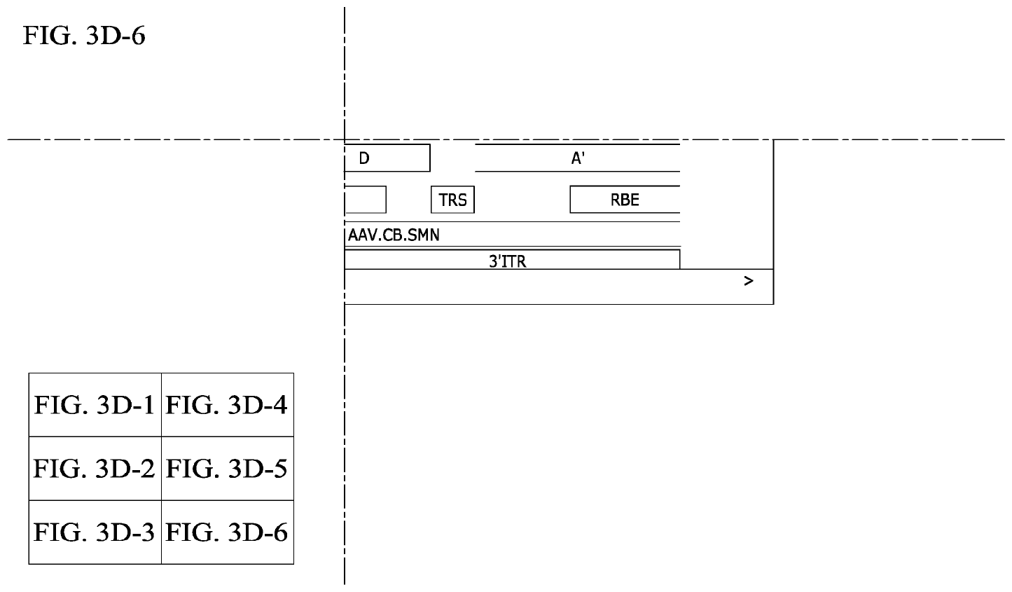

RT-PCR results are shown in FIG. 2A for sc rAAV-U7 constructs with three different antisense sequences (for example, ESE8_2, ESE7, SD6 (combination recited as “136”) or ESE8, SD7, and SD6 (combination shown as “256”). The U7 ese8_2 ese7 sd6 constructs were included in a vector genome, either in the forward or reverse direction (FIG. 3A-D). Each U7 construct carried an antisense directed against exon 6, 7 or 8 (shown in a plasmid map as set out in FIG. 3A-D) comprising in sequence three exons 6, 7 and 8-targeted U7 snRNA polynucleotide constructs: a U7 ESE8_2/ESE8 construct, a U7 ESE7/SD7 construct and a U7 SD6 construct. The U7_U7 ESE8_2/ESE8, SD7/ESE7, SD6 FORWARD OR REVERSE SC rAAV (abbreviated U7_U7 ESE8_2 SD7 SD6 FORWARD OR REVERSE SC rAAV elsewhere herein) achieved a higher percentage of exons 6, 7 and 8 skipping.

In subsequent experiments, exon-skipping efficiency was analyzed in vivo in the hdmd7 mouse model described herein above. The most efficient AAV-U7 vector, rAAV1.U7_U7 ESE8_2 SD7 SD6 FORWARD OR REVERSE which comprises antisense sequences targeting each of exons 6, 7, and 8 was chosen for initial intramuscular injection in hDMDm7 mice.

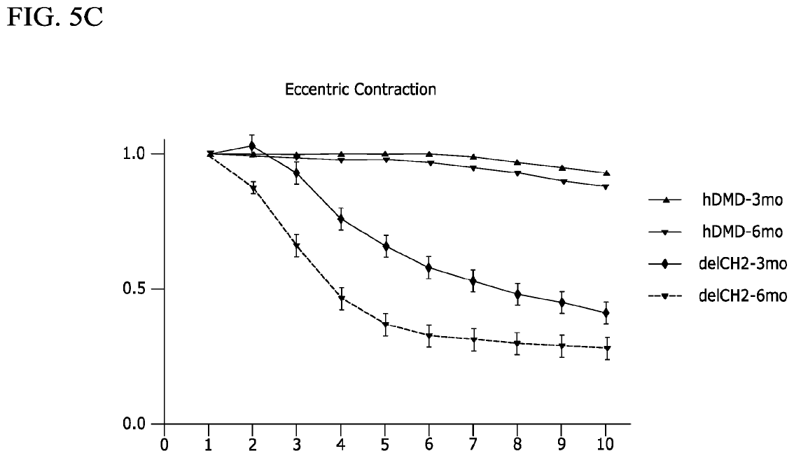

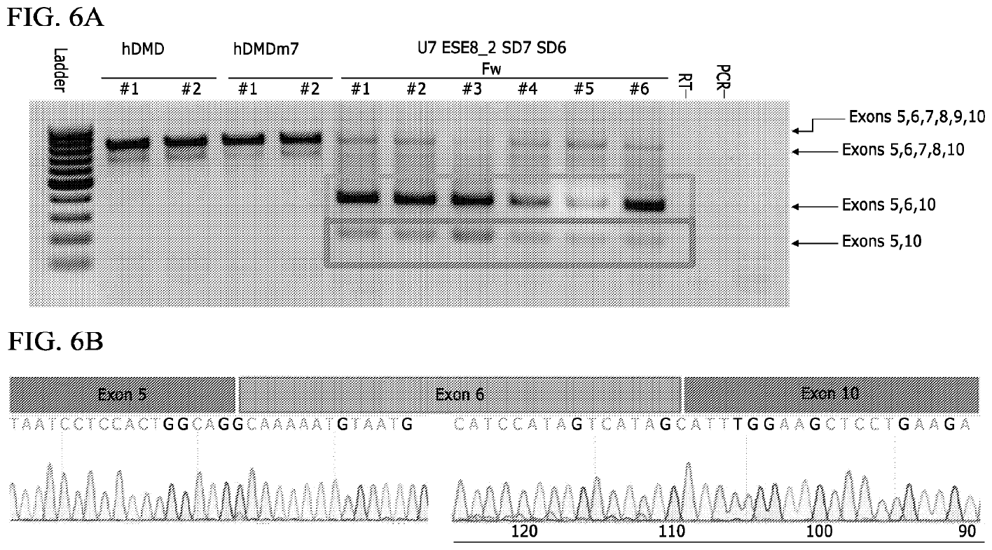

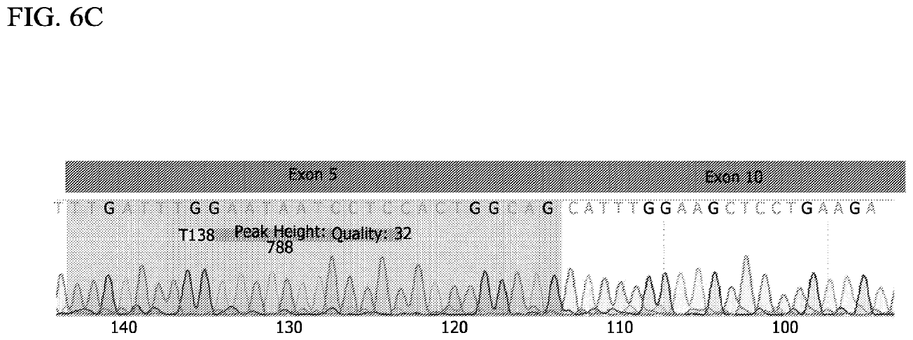

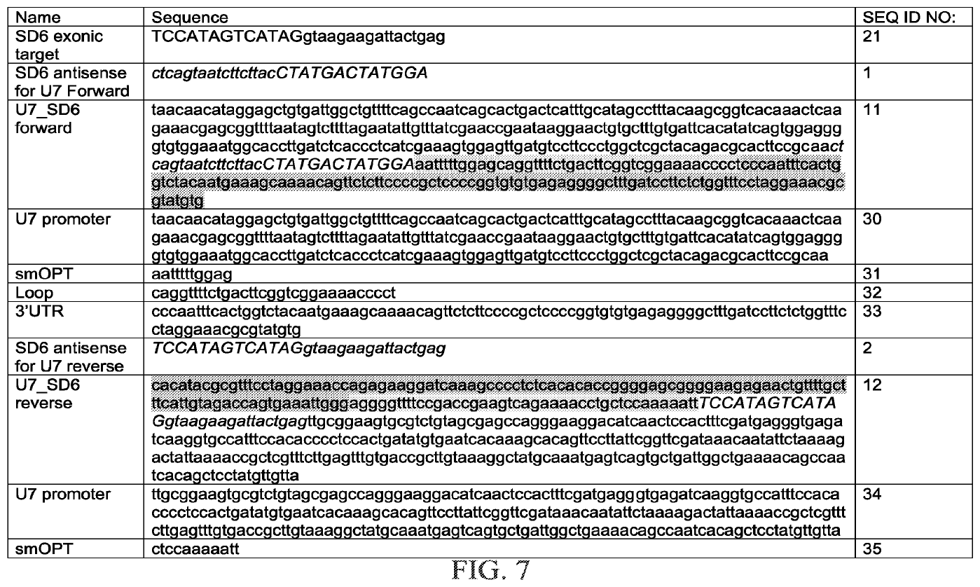

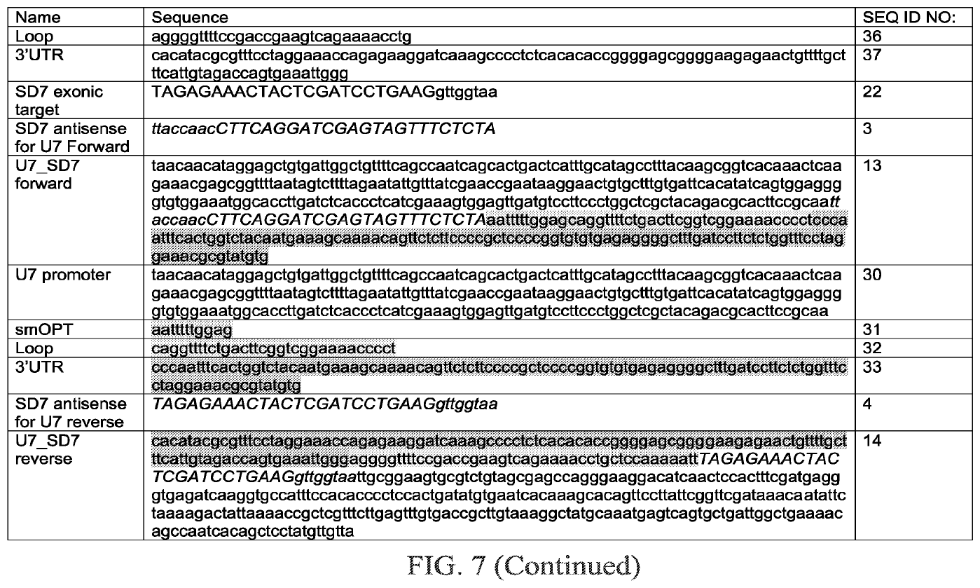

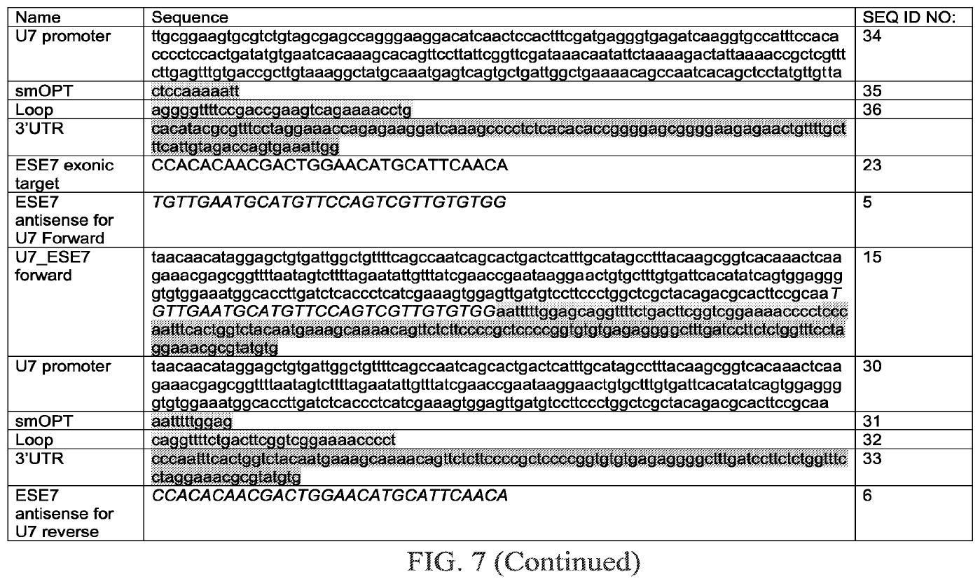

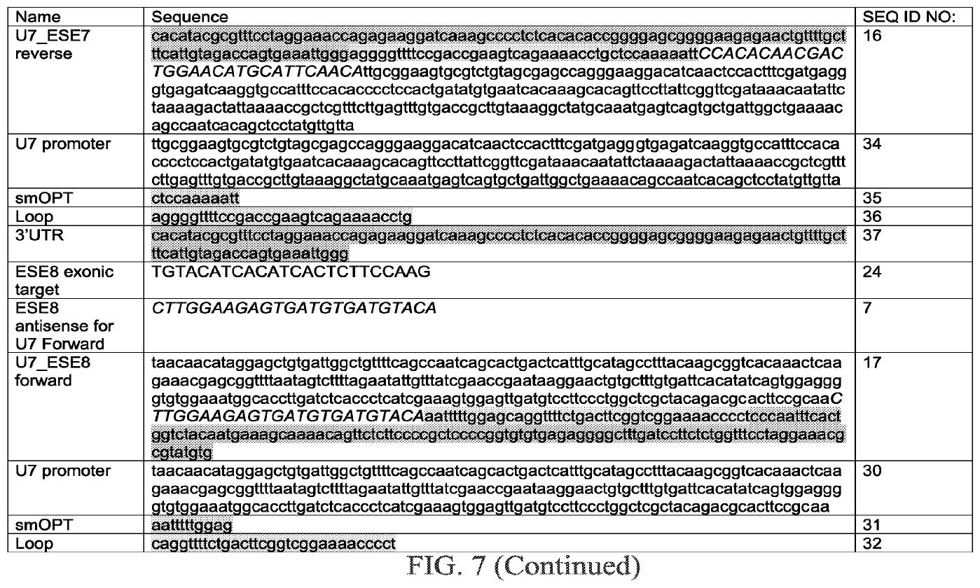

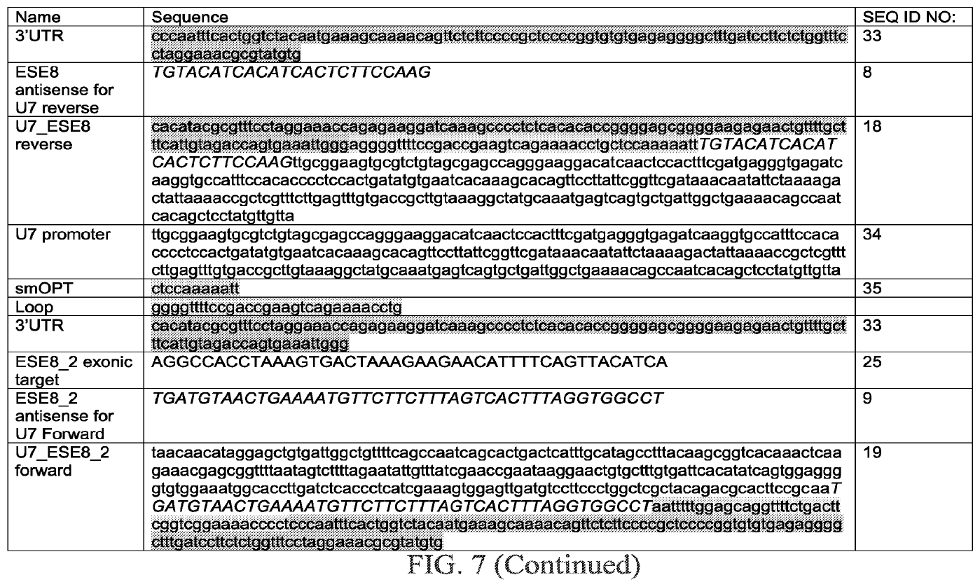

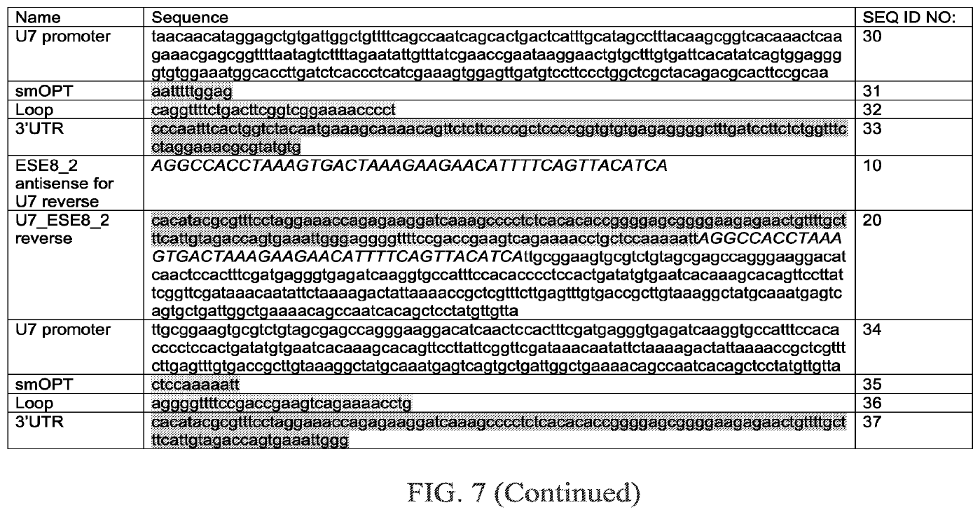

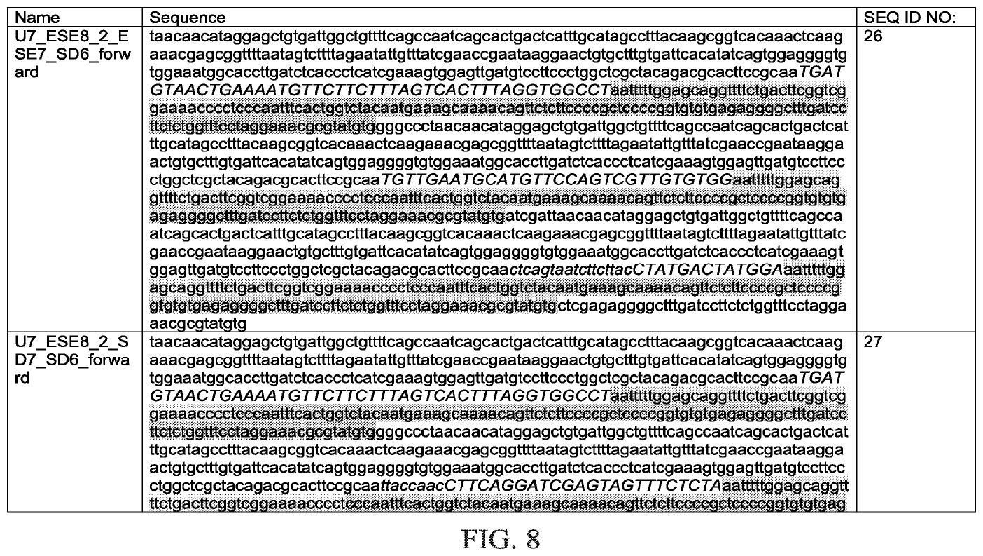

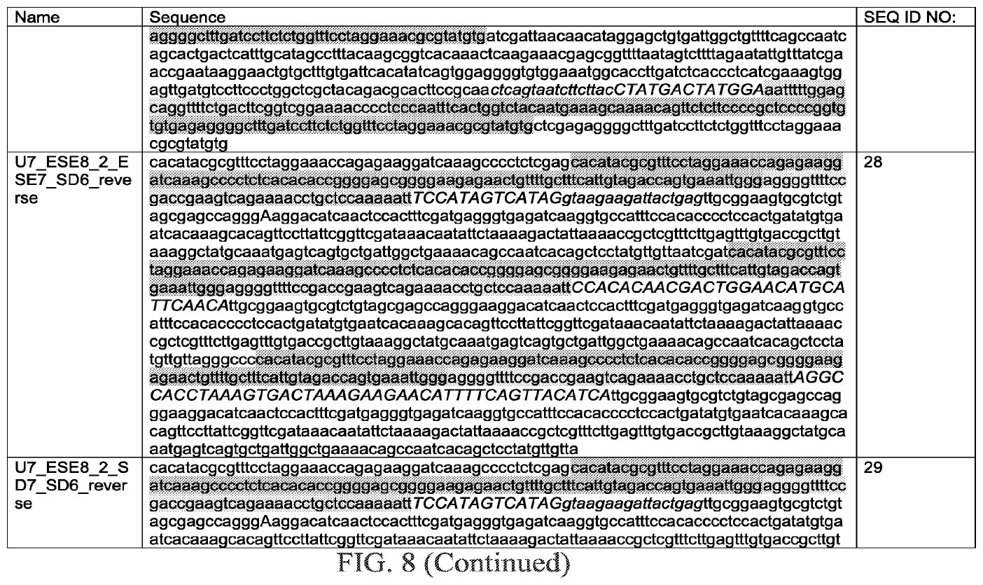

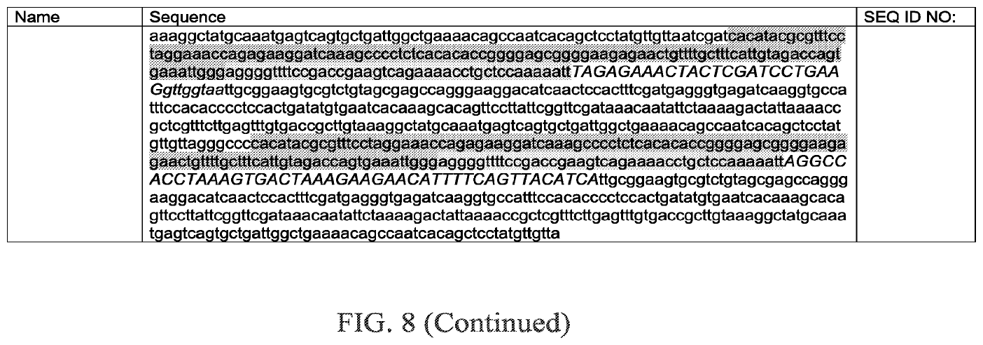

Results are shown in FIG. 6A-C. FIG. 6A shows RT-PCR results of exon skipping of exons 7, 8, and 9 (green box (upper box in FIG. 6A)) or exons 6, 7, 8, and 9 (red box (lower box in FIG. 6A)). Lanes 1-2 show natural amplification in WT hDMD mouse with a transcript having either exons 5, 6, 7, 8, 9, and 10 or exons 5, 6, 7, 8, and 10. Lanes 3-4 show natural amplification in hDMDm7 mouse with the same transcripts. Lanes 5-10 show results of skipping following treatment with U7 ESE8_2 SD7 SD6 Forward. Exons 5, 6, and 10 (green box (upper box in FIG. 6A)) and exons 5 and 10 (red box (lower box in FIG. 6A)) are present confirming the efficiency of skipping. FIG. 6B shows Sanger sequencing confirming the skipping of exons 7, 8, and 9 (green box (upper box in FIG. 6A)) and exons 6, 7, 8, and 9 (red box (lower box in FIG. 6A)) in both transcripts. The transcript sequenced in FIG. 6B is lacking exons 7, 8, and 9 and the transcript sequenced in FIG. 6C is lacking exons 6, 7, 8, and 9. These results confirmed that vectors comprising the antisense sequences were able to mediate exon skipping of exons 6, 7, 8, and 9. These results are slightly different from the in vitro data obtained from FIG. 2A where exon 6 was excluded but exon 9 was present in one of the transcripts following exon skipping. The transcript containing exon 6, but missing exons 7, 8 and/or 9, does not restore the reading frame; however, the transcript containing exons 5, 9 and 10 and the transcript containing exons 5 and 10 both restore the reading frame of DMD.

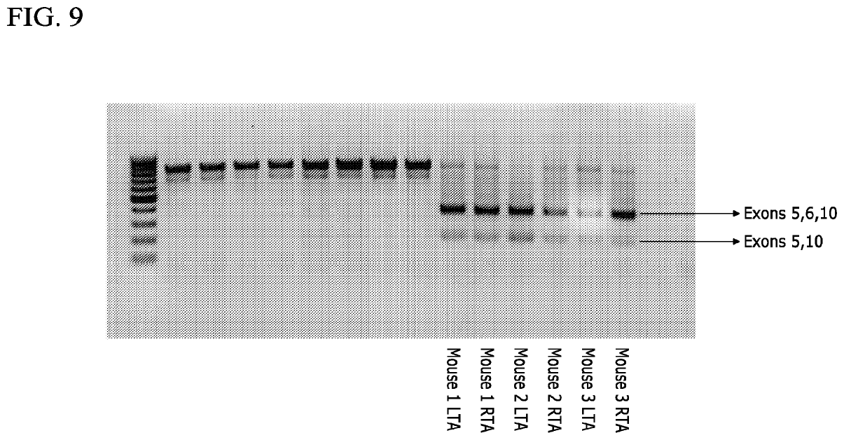

FIG. 9 shows the efficacy of exon skipping following intramuscular injection of three hDMDm7 mice into the left tibialis anterior (LTA) and right tibialis anterior (RTA). Mice were injected with scAAV1.nESE8_SD7_SD6 (TT745-3) designed to skip exons 6, 7, and 8. Results are from RT-PCR conducted on RNA extracted from treated muscle. Bands sequence were confirmed by Sanger sequencing. Transcripts with exon 5, 6, 10 and exons 5, 10 are in-frame and, thus, therapeutic. To conclude, scAAV1.nESE8_SD7_SD6 (TT745-3) is able to skip exons 7, 8 and 9 and partial skipping of exon 6. Transcript that omits or misses exons 6, 7, 8 and 9 is therapeutic as it restores the reading frame of dmd.

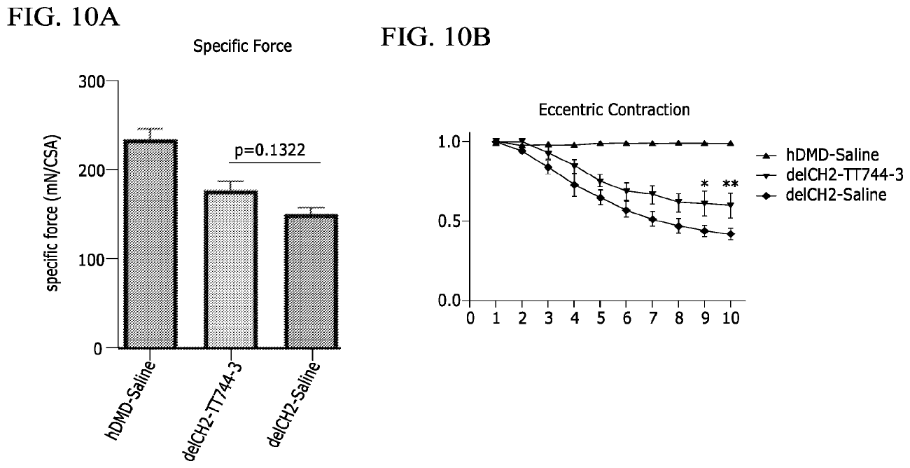

Physiology experiments were performed to measure TA muscle force three months after injection into mice. FIG. 10A-B show results from hDMDm7 mice receiving intramuscular injections of AAV.U7snRNA (construct TT744-3) into both TA muscles at 7 weeks of age. FIG. 10A shows results from measures of specific force in healthy control mice(hDMD-Saline), delCH2/hDMDm7-treated mice (delCH2-TT744-3), and delCH2/hDMDm7 untreated mice (delCH2-Saline), respectively (L to R). FIG. 10B shows results from measures of force from consecutive eccentric contractions from the same groups of mice. TT745-3 was able to ameliorate the specific force of the tibialis anterior and ameliorate the resistance to eccentric force of the tibialis anterior, supporting the therapeutic benefit of this vector.

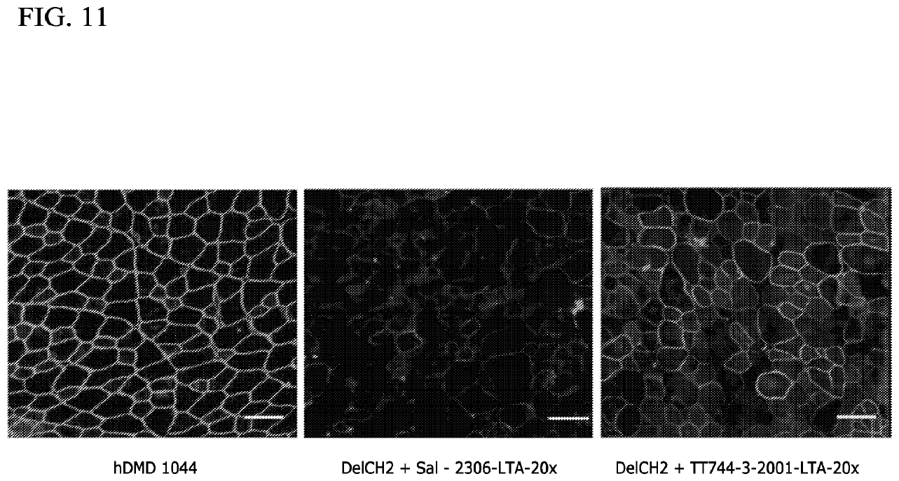

Dystrophin expression in mouse muscle was examined after treatment. FIG. 11 shows representative images of 3-month-old hDMDm7 (referred to as DelCH2 in the image) mice treated (AAV.U7snRNA (construct TT744-3); see far right panel labeled “DelCH2+TT744-3-2001-LTA-20x”) and untreated (saline; see middle panel labeled “DelCH2+Sal−2306-LTA-20x”). A healthy control mouse (hDMD 1044) is shown in the far left panel for comparison. Dystrophin is stained in red via immunofluorescent staining. Images are at 20× magnification of the LTA. These results show that treatment with the TT744-3 construct introduced dystrophin expression in the tibialis anterior three months post injection.

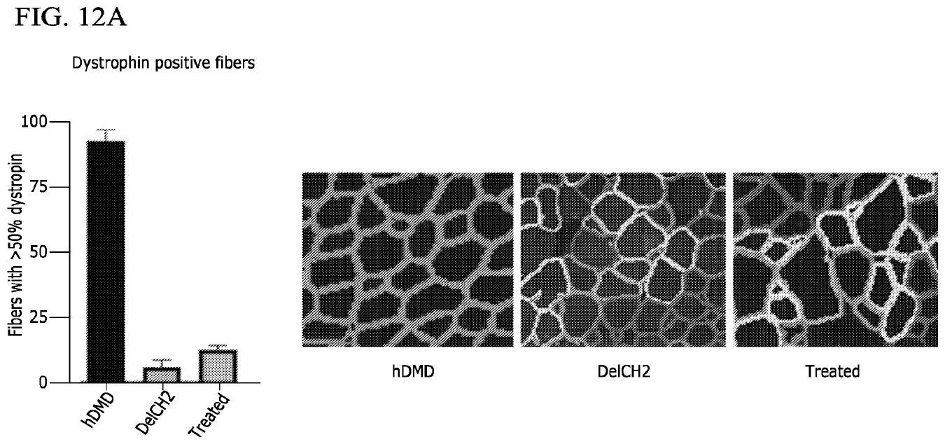

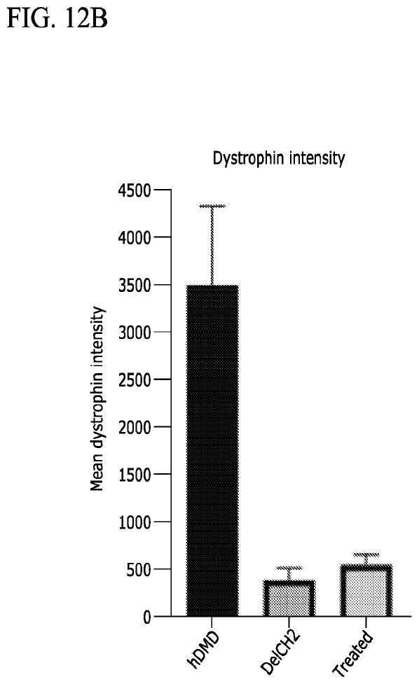

Quantification of dystrophin expression in mice after treatment also was carried out. FIG. 12A-B show quantification of immunofluorescent staining for dystrophin from 20x magnified images of saline on therapeutic injections of mouse LTA and RTA. FIG. 12A shows automated fiber quantification for fibers with more than 50% dystrophin in the treated mouse compared to the untreated mouse (DelCH2). Compared to the WT, the treated mice have about ˜15 dystrophin positive fibers that has more than 50% dystrophin intensity. FIG. 12B shows automated quantification of dystrophin intensity. The intensity of treated mice is overall ˜550 vs untreated mice is overall ˜480, supporting the fact that this construct allow more dystrophin expression post treatment. Altogether, these results show that treatment with the TT744-3 construct introduced dystrophin expression in the tibialis anterior three months post injection.

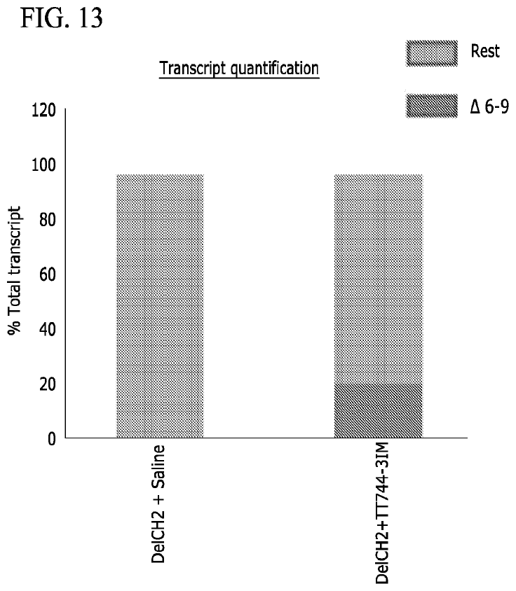

Quantification of percentage of transcripts from hDMDm7/DelCH2 mice injected intramuscularly with saline (left bar) or construct TT744-3 (right bar) also was carried out (FIG. 13). Treatment demonstrated 25% of skipping (in blue corresponding to Δ6-9, the therapeutic transcript) and 75% of none skipped transcript (in orange corresponding to rest, the none skipped transcript). Altogether, this data shows the efficacy of TT744-3 to mediate skipping of exon 6, 7, 8 and 9 which restores the reading frame of dmd allowing dystrophin expression.

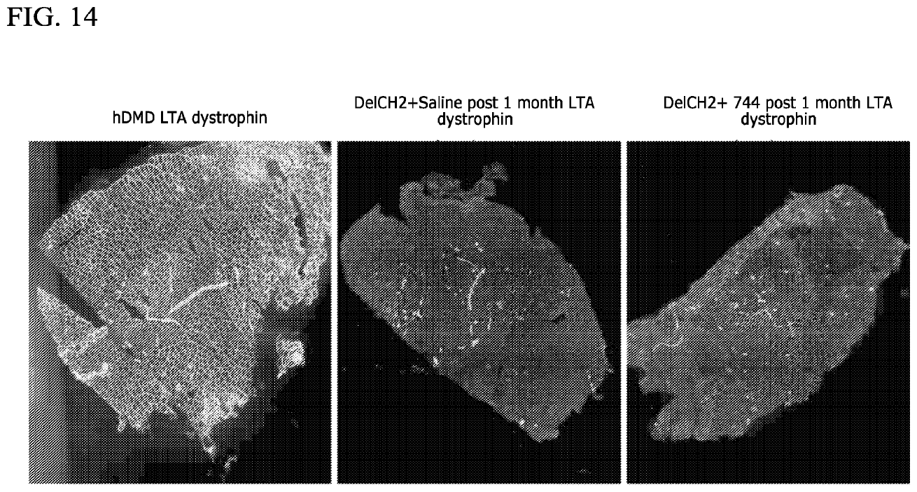

Dystrophin expression five months post-injection was measured. FIG. 14 shows total LTA images (10×) from three mice immunofluorescently stained for dystrophin. hDMD LTA dystrophin (far left panel) shows dystrophin staining in a 5-month-old mouse and acts as a positive control. Staining of LTA for dystrophin 5 months post-injection (DelCH2+Saline; middle panel) shows almost no dystrophin expression. Staining of LTA for dystrophin 5 months post injection (DelCH2+TT744-3; right panel) shows around ˜40% of dystrophin positive fibers. These results show that treatment with TT744-3 restores dystrophin expression in 40% of fibers supporting its therapeutic potential.

### Example 4

### Expression of Dystrophin After Treatment of Human Patients

As described herein above, a therapeutic exon skipping viral vector comprising three U7snRNA containing antisense sequences targeting exons 6, 7, and 8 of the DMD gene was created. Following dose finding studies in mice and after demonstrating lack of toxicity in non-human primates, a first-in-human clinical is initiated. As discussed herein above, if the targeted exons are skipped, a functional form of dystrophin is expressed by this therapy.

While the disclosure has been described in terms of specific aspects, it is understood that variations and modifications will occur to those skilled in the art. Accordingly, only such limitations as appear in the claims should be placed on the disclosure.

All documents referred to in this application are hereby incorporated by reference in their entirety.

We claim:

1. A nucleic acid comprising a nucleotide sequence selected from the group consisting of:
(a) a nucleotide sequence comprising at least 80% identity to the sequence set forth in any one of SEQ ID NOs: 1-20;
(b) a nucleotide sequence complementary to the nucleotide sequence comprising at least 80% identity to the sequence set forth in any one of SEQ ID NOs: 1-20;
(c) a nucleotide sequence comprising the sequence set forth in any one of SEQ ID NOs: 1-20;
(d) a nucleotide sequence complementary to the nucleotide sequence comprising the sequence set forth in any one of SEQ ID NOs: 1-20; and
(e) a nucleotide sequence which binds to the sequence set forth in any one of SEQ ID NOs: 21-25.

2. The nucleic acid of claim 1 comprising a combination of at least two nucleotide sequences, wherein the combination comprises
(i) a nucleotide sequence that targets the human DMD gene at exon 6 and a nucleotide sequence that targets the human DMD gene at exon 7,
(ii) a nucleotide sequence that targets the human DMD gene at exon 6 and a nucleotide sequence that targets the human DMD gene at exon 8, and
(iii) a nucleotide sequence that targets the human DMD gene at exon 7 and a nucleotide sequence that targets the human DMD gene at exon 8.

3. The nucleic acid of claim 1 or 2 comprising a combination of at least three nucleotide sequences, wherein the combination comprises a nucleotide sequence that targets the human DMD gene at exon 6, a nucleotide sequence that targets the human DMD gene at exon 7, and a nucleotide sequence that targets the human DMD gene at exon 8.

4. The nucleic acid of any one of claims 1-3 comprising a nucleotide sequence selected from the group consisting of:
(a) a nucleotide sequence comprising at least 70% identity to the sequence set forth in any one of SEQ ID NOs: 26-29;
(b) a nucleotide sequence complementary to the nucleotide sequence comprising at least 70% identity to the sequence set forth in any one of SEQ ID NOs: 26-29;
(c) a nucleotide sequence comprising the sequence set forth in any one of SEQ ID NOs: 26-29; and
(d) a nucleotide sequence complementary to the nucleotide sequence comprising the sequence set forth in any one of SEQ ID NOs: 26-29.

5. A recombinant adeno-virus associated (rAAV) comprising the nucleic acid of any one of claims 1-4.

6. The rAAV of claim 5, wherein the rAAV is rAAV1, rAAV2, rAAV3, rAAV4, rAAV5, rAAV6, rAAV7, rAAV8, rAAV9, rAAV10, rAAV11, rAAV12, rAAV13, rAAV-anc80, rAAV rh.74, rAAV rh.8, rAAVrh.10, or rAAV-B1.

7. The rAAV of claim 5 or 6, wherein the rAAV is rAAV9.

8. The rAAV of any one of claims 5-7, wherein the rAAV is self-complementary.

9. A composition comprising the nucleic acid of any one of claims 1-4 and a carrier, diluent, excipient, and/or adjuvant.

10. A composition comprising the rAAV of any one of claims 5-8 and a carrier, diluent, excipient, and/or adjuvant.

11. A method of treating, preventing or ameliorating a muscular dystrophy in a subject in need thereof comprising the step of administering to the subject an effective amount of
(a) the nucleic acid of any one of claims 1-4;
(b) the rAAV of any one of claims 5-8; or
(c) the composition of claim 9 or 10.

12. The method of claim 11, wherein the administering is via a systemic route.

13. The method of claim 12, wherein the systemic route is by injection, infusion or implantation.

14. The method of any one of claims 10-13, wherein the muscular dystrophy is Duchenne Muscular Dystrophy or Becker Muscular Dystrophy.

15. The method of any one of claims 10-14, wherein the level of functional dystrophin gene expression or protein expression in a cell of the subject is increased after administering the nucleic acid, rAAV, or composition as compared to the level of functional dystrophin gene expression or protein expression before administering the nucleic acid, rAAV, or composition.

16. The method of claim 15, wherein expression of functional dystrophin in the cell is detected by measuring the dystrophin protein level by Western blot, immunofluorescence, or immunohistochemistry in muscle biopsied before and after administering the nucleic acid, rAAV, or composition.

17. The method of any one of claims 10-15, wherein the level of serum creatinine kinase is decreased after administering the nucleic acid, rAAV, or composition as compared to the level of serum creatinine kinase before administering the nucleic acid, rAAV, or composition.

18. The method of any one of claims 10-15 which results in improved muscle strength, improved muscle function, improved mobility, improved stamina, or a combination of two or more thereof in the subject.

19. The method of any one of claims 10-15, wherein muscular dystrophy progression in the subject is delayed or wherein muscle function in the subject is improved after administering the nucleic acid, rAAV, or composition as measured by the six minute walk test, time to rise test, ascend 4 steps test, ascend and descend 4 steps test, North Star Ambulatory Assessment (NSAA), the forced vital capacity (FVC) test, 10 meter timed test, 100 meter timed test, hand held dynamometry (HHD) test, Timed Up and Go test, Gross Motor Subtest Scaled (Bayley-III) score, maximum isometric voluntary contraction test (MVICT), or a combination of two or more thereof.

20. The method of any one of claims 10-15 further comprising administering a second or combination therapy.

21. The method of claim 20 comprising administering a glucocorticoid.

22. Use of the
(a) the nucleic acid of any one of claims 1-4;
(b) the rAAV of any one of claims 5-8; or
(c) the composition of claim 9 or 10
for the preparation of a medicament for the treatment of a muscular dystrophy, or
for treating a muscular dystrophy in a human subject in need thereof.

23. The use of claim 22, wherein treating is via a systemic route.

24. The use of claim 23, wherein the systemic route is by injection, infusion or implantation.

25. The use of any one of claims 22-24, wherein the muscular dystrophy is Duchenne Muscular Dystrophy or Becker Muscular Dystrophy.

26. The use of any one of claims 22-25, wherein the level of functional dystrophin gene expression or protein expression in a cell of the subject is increased after use of the nucleic acid, rAAV, or composition as compared to the level of functional dystrophin gene expression or protein expression before the use of the nucleic acid, rAAV, or composition.

27. The use of claim 26, wherein expression of functional dystrophin in the cell is detected by measuring the dystrophin protein level by Western blot, immunofluorescence, or immunohistochemistry in muscle biopsied before and after administering the nucleic acid, rAAV, or composition.

28. The use of any one of claims 22-25, wherein the level of serum creatinine kinase is decreased after administering the nucleic acid, rAAV, or composition as compared to the level of serum creatinine kinase before administering the nucleic acid, rAAV, or composition.

29. The use of any one of claims 22-25 which results in improved muscle strength, improved muscle function, improved mobility, improved stamina, or a combination of two or more thereof in the subject.

30. The use of any one of claims 22-25, wherein muscular dystrophy progression in the subject is delayed or wherein muscle function in the subject is improved after administering the nucleic acid, rAAV, or composition as measured by the six minute walk test, time to rise test, ascend 4 steps test, ascend and descend 4 steps test, North Star Ambulatory Assessment (NSAA), the forced vital capacity (FVC) test, 10 meter timed test, 100 meter timed test, hand held dynamometry (HHD) test, Timed Up and Go test, Gross Motor Subtest Scaled (Bayley-III) score, maximum isometric voluntary contraction test (MVICT), or a combination of two or more thereof.

31. The use of any one of claims 22-25 further comprising the use of a second or combination therapy.

32. The use of claim 21 comprising the use of a glucocorticoid.


SEQUENCE LISTING (37 sequences)

SEQ ID NO: 1 - DNA, 30 bp, Artificial Sequence
  Synthetic
ctcagtaatcttcttacctatgactatgga

SEQ ID NO: 2 - DNA, 30 bp, Artificial Sequence
  Synthetic
tccatagtcataggtaagaagattactgag

SEQ ID NO: 3 - DNA, 32 bp, Artificial Sequence
  Synthetic
ttaccaaccttcaggatcgagtagtttctcta

SEQ ID NO: 4 - DNA, 32 bp, Artificial Sequence
  Synthetic
tagagaaactactcgatcctgaaggttggtaa

SEQ ID NO: 5 - DNA, 30 bp, Artificial Sequence
  Synthetic
tgttgaatgcatgttccagtcgttgtgtgg

SEQ ID NO: 6 - DNA, 30 bp, Artificial Sequence
  Synthetic
ccacacaacgactggaacatgcattcaaca

SEQ ID NO: 7 - DNA, 24 bp, Artificial Sequence
  Synthetic
cttggaagagtgatgtgatgtaca

SEQ ID NO: 8 - DNA, 24 bp, Artificial Sequence
  Synthetic
tgtacatcacatcactcttccaag

SEQ ID NO: 9 - DNA, 44 bp, Artificial Sequence
  Synthetic
tgatgtaactgaaaatgttcttctttagtcactttaggtggcct

SEQ ID NO: 10 - DNA, 44 bp, Artificial Sequence
  Synthetic
aggccacctaaagtgactaaagaagaacattttcagttacatca

SEQ ID NO: 11 - DNA, 437 bp, Artificial Sequence
  Synthetic
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaactcagtaatcttcttacctatgactatggaaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 12 - DNA, 437 bp, Artificial Sequence
  Synthetic
cacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaatttccatagtcataggtaagaagattactgagttgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgtta

SEQ ID NO: 13 - DNA, 439 bp, Artificial Sequence
  Synthetic
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaattaccaaccttcaggatcgagtagtttctctaaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 14 - DNA, 439 bp, Artificial Sequence
  Synthetic
cacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaatttagagaaactactcgatcctgaaggttggtaattgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgtta

SEQ ID NO: 15 - DNA, 437 bp, Artificial Sequence
  Synthetic
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaatgttgaatgcatgttccagtcgttgtgtggaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 16 - DNA, 437 bp, Artificial Sequence
  Synthetic
cacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaattccacacaacgactggaacatgcattcaacattgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgtta

SEQ ID NO: 17 - DNA, 431 bp, Artificial Sequence
  Synthetic
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaacttggaagagtgatgtgatgtacaaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 18 - DNA, 431 bp, Artificial Sequence
  Synthetic
cacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaatttgtacatcacatcactcttccaagttgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgtta

SEQ ID NO: 19 - DNA, 451 bp, Artificial Sequence
  Synthetic
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaatgatgtaactgaaaatgttcttctttagtcactttaggtggcctaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtg

SEQ ID NO: 20 - DNA, 451 bp, Artificial Sequence
  Synthetic
cacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaattaggccacctaaagtgactaaagaagaacattttcagttacatcattgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgtta

SEQ ID NO: 21 - DNA, 30 bp, Artificial Sequence; identical to SEQ ID NO: 2
  Synthetic

SEQ ID NO: 22 - DNA, 32 bp, Artificial Sequence; identical to SEQ ID NO: 4
  Synthetic

SEQ ID NO: 23 - DNA, 30 bp, Artificial Sequence; identical to SEQ ID NO: 6
  Synthetic

SEQ ID NO: 24 - DNA, 24 bp, Artificial Sequence
  Synthetic
tgtacatcacatcactcttccaag

SEQ ID NO: 25 - DNA, 44 bp, Artificial Sequence; identical to SEQ ID NO: 10
  Synthetic

SEQ ID NO: 26 - DNA, 1387 bp, Artificial Sequence
  Synthetic
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaatgatgtaactgaaaatgttcttctttagtcactttaggtggcctaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtggggccctaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaatgttgaatgcatgttccagtcgttgtgtggaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgatcgattaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaactcagtaatcttcttacctatgactatggaaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgctcgagaggggctttgatccttctctggtttcctaggaaacgcgtatgtg

SEQ ID NO: 27 - DNA, 1389 bp, Artificial Sequence
  Synthetic
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaatgatgtaactgaaaatgttcttctttagtcactttaggtggcctaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtggggccctaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaattaccaaccttcaggatcgagtagtttctctaaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgatcgattaacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaactcagtaatcttcttacctatgactatggaaatttttggagcaggttttctgacttcggtcggaaaacccctcccaatttcactggtctacaatgaaagcaaaacagttctcttccccgctccccggtgtgtgagaggggctttgatccttctctggtttcctaggaaacgcgtatgtgctcgagaggggctttgatccttctctggtttcctaggaaacgcgtatgtg

SEQ ID NO: 28 - DNA, 1387 bp, Artificial Sequence
  Synthetic
cacatacgcgtttcctaggaaaccagagaaggatcaaagcccctctcgagcacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaatttccatagtcataggtaagaagattactgagttgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgttaatcgatcacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaattccacacaacgactggaacatgcattcaacattgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgttagggccccacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaattaggccacctaaagtgactaaagaagaacattttcagttacatcattgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgtta

SEQ ID NO: 29 - DNA, 1389 bp, Artificial Sequence
  Synthetic
cacatacgcgtttcctaggaaaccagagaaggatcaaagcccctctcgagcacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaatttccatagtcataggtaagaagattactgagttgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgttaatcgatcacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaatttagagaaactactcgatcctgaaggttggtaattgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgttagggccccacatacgcgtttcctaggaaaccagagaaggatcaaagcccctctcacacaccggggagcggggaagagaactgttttgctttcattgtagaccagtgaaattgggaggggttttccgaccgaagtcagaaaacctgctccaaaaattaggccacctaaagtgactaaagaagaacattttcagttacatcattgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgtta

SEQ ID NO: 30 - DNA, 258 bp, Artificial Sequence
  Synthetic
taacaacataggagctgtgattggctgttttcagccaatcagcactgactcatttgcatagcctttacaagcggtcacaaactcaagaaacgagcggttttaatagtcttttagaatattgtttatcgaaccgaataaggaactgtgctttgtgattcacatatcagtggaggggtgtggaaatggcaccttgatctcaccctcatcgaaagtggagttgatgtccttccctggctcgctacagacgcacttccgcaa

SEQ ID NO: 31 - DNA, 11 bp, Artificial Sequence
  Synthetic
aatttttggag

SEQ ID NO: 32 - DNA, 31 bp, Artificial Sequence
  Synthetic
caggttttctgacttcggtcggaaaacccct

SEQ ID NO: 33 - DNA, 107 bp, Artificial Sequence
  Synthetic
cccaatttcactggtctacaatgaaagcaaaacagttctcttccccgctccccggtgtgtgagaggggctttgatccttctctggtttcctaggaaacgcgtatgtg

SEQ ID NO: 34 - DNA, 258 bp, Artificial Sequence
  Synthetic
ttgcggaagtgcgtctgtagcgagccagggaaggacatcaactccactttcgatgagggtgagatcaaggtgccatttccacacccctccactgatatgtgaatcacaaagcacagttccttattcggttcgataaacaatattctaaaagactattaaaaccgctcgtttcttgagtttgtgaccgcttgtaaaggctatgcaaatgagtcagtgctgattggctgaaaacagccaatcacagctcctatgttgtta

SEQ ID NO: 35 - DNA, 11 bp, Artificial Sequence
  Synthetic
ctccaaaaatt

SEQ ID NO: 36 - DNA, 31 bp, Artificial Sequence
  Synthetic
aggggttttccgaccgaagtcagaaaacctg

SEQ ID NO: 37 - DNA, 107 bp, Artificial Sequence
  Synthetic
cacatacgcgtttcctaggaaaccagagaaggatcaaagcccctctcacacaccggggagcggggaagagaactgttttgctttcattgtagaccagtgaaattggg